(19) United States
(12) Patent Application Publication
(10) Pub. No.: US 2025/0197396 A1
(43) Pub. Date: Jun. 19, 2025

(54) COMPOUNDS FOR INHIBITING INOSITOL HEXAKISPHOSPHATE KINASE (IP6K) AND METHODS OF USE THEREOF

(71) Applicant: Lieber Institute, Inc., Baltimore, MD (US)

(72) Inventors: Glen Ernst, Bear, DE (US); James Barrow, Arnold, MD (US); Huijun Wei, Potomac, MD (US)

(21) Appl. No.: 18/256,092

(22) PCT Filed: Dec. 7, 2021
(86) PCT No.: PCT/US21/62169

§ 371 Date: Jun. 6, 2023

Related U.S. Application Data
(60) Provisional application No. 63/122,197, filed on Dec. 7, 2020.

(51) Int. Cl. C07D 471/10; A61K 31/4184; A61K 31/438; A61K 31/4439; A61K 31/444; A61K 31/454; C07D 235/26; C07D 401/04; C07D 401/12; C07D 403/04; C07D 403/06

(52) U.S. Cl. CPC C07D 471/10; A61K 31/4184; A61K 31/438; A61K 31/4439; A61K 31/444; A61K 31/454; C07D 235/26; C07D 401/04; C07D 401/12; C07D 403/04; C07D 403/06

(57) ABSTRACT

Compounds for inhibiting IP6K and methods for treating a condition, disease, or disorder associated with IP6K activity or expression are disclosed.

### FEDERALLY SPONSORED RESEARCH OR DEVELOPMENT

This invention was made with government support under MH112658 awarded by the National Institutes of Health. The government has certain rights in the invention.

### BACKGROUND

In type 2 diabetes (T2D), skeletal, hepatic and adipose insulin resistance are all traceable to defects of insulin signaling including the insulin receptor (INSR), insulin receptor substrates (IRS1), phosphoinositide 3-kinase (PI3K), and AKT activity. Higher-order inositol pyrophosphates such as 5-diphospho-inositol pentakisphosphate (IP7) can bind and inhibit AKT activation. Inhibition of IP7 production by deletion or inhibition of inositol hexakisphosphate kinase (IP6K) has been shown to increase AKT phosphorylation, increase insulin sensitivity and lower blood glucose. In addition, inhibition of IP6K increases mitochondria biogenesis, mitochondria activity and cellular ATP levels, all of which are known to be reduced in T2D patients. Impaired AKT signaling through a downstream target glycogen synthesis kinase 3 (GSK3) also has been implicated in bipolar disorder and other neuropsychiatric conditions. Therefore, modulating the IP6K-AKT-GSK3 interaction may exert beneficial effects in psychiatric disorders involving GSK3.

### SUMMARY

In some aspects, the presently disclosed subject matter provides a compound of Formula (IA):

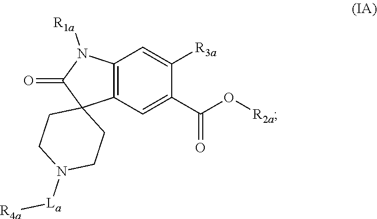

wherein:
R₁ₐ and R₂ₐ are each independently selected from the group consisting of H and substituted or unsubstituted branched or straightchain C₁-C₄ alkyl, provided that at least one of R₁ₐ and R₂ₐ is H;
R₃ₐ is H or halogen;
R₄ₐ is selected from the group consisting of substituted or unsubstituted branched or straightchain C₁-C₄ alkyl, substituted or unsubstituted cycloalkyl, substituted or unsubstituted cycloheteroalkyl, substituted or unsubstituted aryl, and substituted or unsubstituted heteroaryl;
Lₐ is selected from the group consisting of —(CH₂)ₙ—, —S(═O)₂—, —C(═O)—(CH₂)ₘ—, —C(═O)—NH—, and —C(═O)—(CH₂)ₚ—O—;
wherein n and p are each integers selected from 1, 2, 3, and 4; m is 0 or 1; and
stereoisomers and pharmaceutically acceptable salts thereof.

In some aspects, R₁ₐ and R₂ₐ are each independently H or methyl, provided that at least one of R₁ₐ and R₂ₐ is H.

In some aspects, R₃ₐ is H or F.

In some aspects, R₄ₐ is selected from the group consisting of methyl, isopropyl, cyclopropyl, substituted or unsubstituted phenyl, substituted or unsubstituted pyridyl, and substituted or unsubstituted pyrimidinyl.

In certain aspects, the phenyl, pyridinyl, or pyrimidinyl is substituted with one or more substituent groups selected from the group consisting of substituted or unsubstituted branched or straightchain C₁-C₄ alkyl, C₁-C₄ alkoxyl, cyclopropyl, trifluoromethyl, hydroxyl, halogen, cyano, carbamoyl, and benzyloxy.

In particular aspects, R₄ₐ is selected from the group consisting of 2-chlorophenyl, 4-chlorophenyl, 2,4-dichlorophenyl, 4-trifluoromethylphenyl, and 3,5-dichloropyridin-2-yl.

In some aspects, the presently disclosed subject matter provides a compound of Formula (IB):

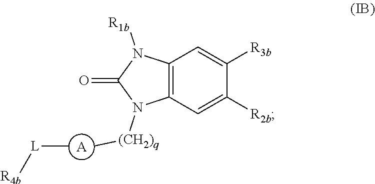

wherein:
q is 0 or 1;
A can be present or absent and when present is a 4-, 5-, or 6-membered cycloalkyl or cycloheteroalkyl ring;
R_{1b} is selected from the group consisting of H and substituted or unsubstituted branched or straightchain C₁-C₄ alkyl;
R_{2b} is H or —C(═O)—OR_{5b};
R_{3b} is selected from the group consisting of H, halogen, and —C(═O)—OR_{5b}, wherein R_{5b} is selected from the group consisting of H and substituted or unsubstituted branched or straightchain C₁-C₄ alkyl, provided that at least one of R_{1b} and R_{5b} is H;
R_{4b} is selected from the group consisting of substituted or unsubstituted branched or straightchain C₁-C₄ alkyl, substituted or unsubstituted cycloalkyl, substituted or unsubstituted cycloheteroalkyl, substituted or unsubstituted aryl, and substituted or unsubstituted heteroaryl;
L_{b} is selected from the group consisting of —C(═O)—, —(CH₂)ᵣ—, and —(CH₂)ₛ—O—; wherein r is selected from the group consisting of 1, 2, 3, or 4 and s is an integer selected from the group consisting of 0, 1, 2, or 3; and
stereoisomers and pharmaceutically acceptable salts thereof.

In certain aspects, the compound of Formula (IB) is:

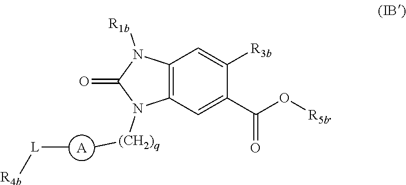

In some aspects, R_{1b} and R_{5b} are each H.

In some aspects, R_{3b} is H or F.

In some aspects, A is present and is selected from the group consisting of azetidinyl, pyrrolidinyl, piperidinyl, and cyclohexyl:

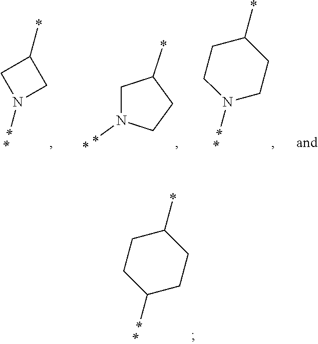

wherein * indicates a point of attachment of ring A to a nitrogen atom in the 3-position of the 2,3-dihydro-1H-benzo[d]imidazole ring and ** indicates a point of attachment of ring A to linker L_{b}.

In some aspects, R₄ₐ is selected from the group consisting of methyl, isopropyl, cyclopropyl, substituted or unsubstituted phenyl, substituted or unsubstituted pyridyl, and substituted or unsubstituted pyrimidinyl.

In certain aspects, the phenyl, pyridinyl, or pyrimidinyl is substituted with one or more substituent groups selected from the group consisting of substituted or unsubstituted branched or straightchain C₁-C₄ alkyl, C₁-C₄ alkoxyl, cyclopropyl, trifluoromethyl, hydroxyl, halogen, cyano, carbamoyl, and benzyloxy.

In particular aspects, R_{4b} is halogen-substituted phenyl or halogen-substituted pyridyl.

In more particular aspects, R_{4b} is selected from the group consisting of 2-chlorophenyl, 4-chlorophenyl, 2,4-dichlorophenyl, and 3,5-dichloropyridin-2-yl.

In other aspects, the presently disclosed subject matter provides a method for treating a disease, condition, or disorder associated with IP6K, the method comprising administering a compound of Formula (IA) or Formula (IB), or a pharmaceutically acceptable salt thereof, to a subject in need of such treatment.

In certain aspects, the disease, condition, or disorder is selected from the group consisting of a psychiatric disease, Alzheimer's disease, and diabetes. In particular aspects, the psychiatric disease is bipolar disorder.

Certain aspects of the presently disclosed subject matter having been stated hereinabove, which are addressed in whole or in part by the presently disclosed subject matter, other aspects will become evident as the description proceeds when taken in connection with the accompanying Examples as best described herein below.

### BRIEF DESCRIPTION OF THE FIGURES

Having thus described the presently disclosed subject matter in general terms, reference will now be made to the accompanying Figures, which are not necessarily drawn to scale, and wherein:

FIG. 1 provides the chemical structures of representative, presently disclosed IP6K inhibitors of Formula (IA); and

FIG. 2 provides the chemical structures of representative, presently disclosed IP6K inhibitors of Formula (IB).

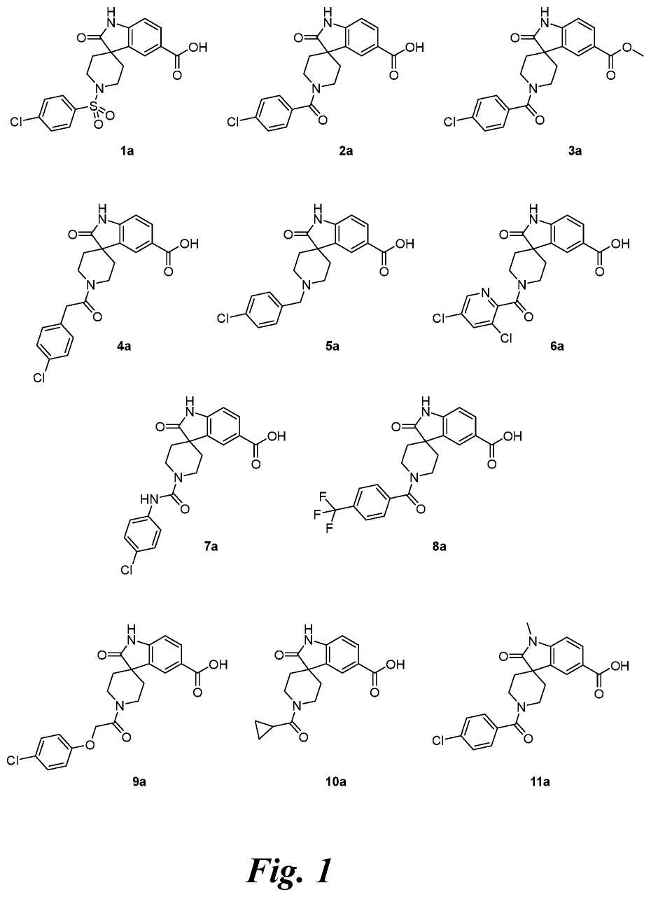

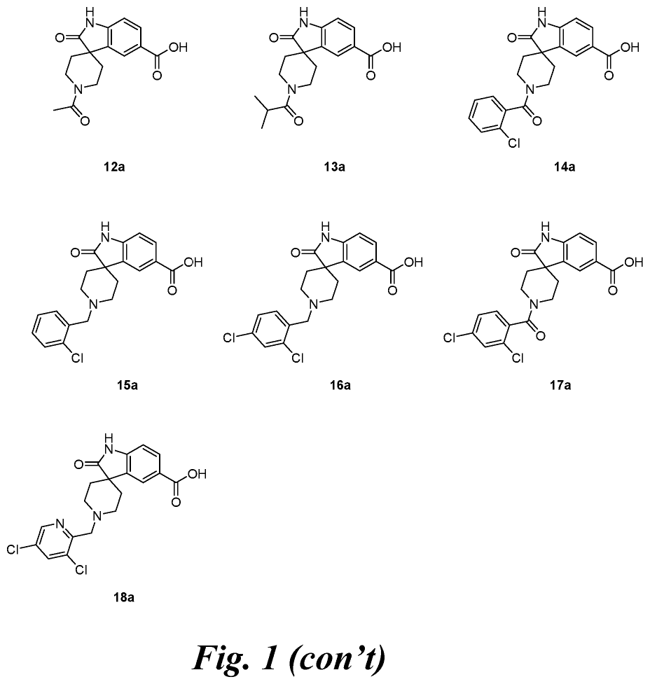

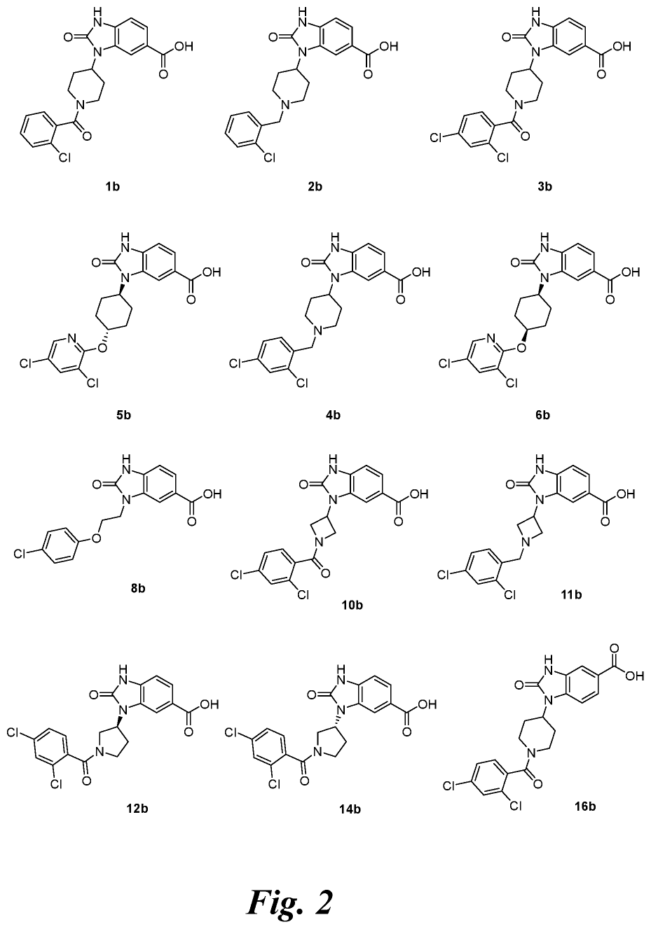

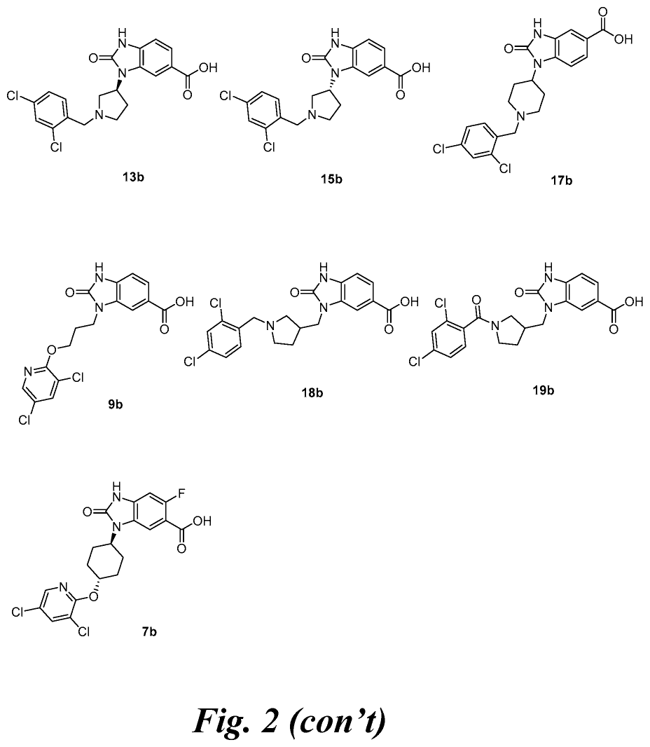

### DETAILED DESCRIPTION

The presently disclosed subject matter now will be described more fully hereinafter. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims.

### I. Compounds for Inhibiting IP6K

The presently disclosed subject matter compounds for inhibiting IP6K and methods of their use for treating diseases, conditions, or disorders associated with IP6K. In some embodiments, the disease, condition, or disorder is associated with an increased IP6K activity or expression.

### A. Representative Compounds of Formula (IA) and Formula (IB)

In some embodiments, the presently disclosed subject matter provides a compound of Formula (IA):

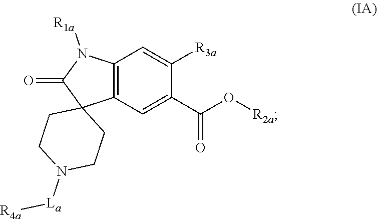

wherein:
R₁ₐ and R₂ₐ are each independently selected from the group consisting of H and substituted or unsubstituted branched or straightchain C₁-C₄ alkyl, provided that at least one of R₁ₐ and R₂ₐ is H;
R₃ₐ is H or halogen;
R₄ₐ is selected from the group consisting of substituted or unsubstituted branched or straightchain C₁-C₄ alkyl, substituted or unsubstituted cycloalkyl, substituted or unsubstituted cycloheteroalkyl, substituted or unsubstituted aryl, and substituted or unsubstituted heteroaryl;
Lₐ is selected from the group consisting of —(CH₂)ₙ—, —S(═O)₂—, —C(═O)—(CH₂)ₘ—, —C(═O)—NH—, and —C(═O)—(CH₂)ₚ—O—;
wherein n and p are each integers selected from 1, 2, 3, and 4; m is 0 or 1; and
stereoisomers and pharmaceutically acceptable salts thereof.

In some embodiments, R₁ₐ and R₂ₐ are each independently H or methyl, provided that at least one of R₁ₐ and R₂ₐ is H.

In some embodiments, R₃ₐ is H or F.

In some embodiments, R₄ₐ is selected from the group consisting of methyl, isopropyl, cyclopropyl, substituted or unsubstituted phenyl, substituted or unsubstituted pyridyl, and substituted or unsubstituted pyrimidinyl.

In certain embodiments, the phenyl, pyridinyl, or pyrimidinyl is substituted with one or more substituent groups selected from the group consisting of substituted or unsubstituted branched or straightchain C₁-C₄ alkyl, C₁-C₄ alkoxyl, cyclopropyl, trifluoromethyl, hydroxyl, halogen, cyano, carbamoyl, and benzyloxy.

In particular embodiments, R₄ₐ is selected from the group consisting of 2-chlorophenyl, 4-chlorophenyl, 2,4-dichlorophenyl, 4-trifluoromethylphenyl, and 3,5-dichloropyridin-2-yl.

In yet more particular embodiments, the compound of Formula (IA) is selected from the group consisting of:
1′-((4-chlorophenyl)sulfonyl)-2-oxospiro[indoline-3,4′-piperidine]-5-carboxylic acid;
1′-(4-chlorobenzoyl)-2-oxospiro[indoline-3,4′-piperidine]-5-carboxylic acid;
methyl 1′-(4-chlorobenzoyl)-2-oxospiro[indoline-3,4′-piperidine]-5-carboxylate;
1′-(2-(4-chlorophenyl)acetyl)-2-oxospiro[indoline-3,4′-piperidine]-5-carboxylic acid;
1′-(4-chlorobenzyl)-2-oxospiro[indoline-3,4′-piperidine]-5-carboxylic acid;
1′-(3,5-dichloropicolinoyl)-2-oxospiro[indoline-3,4′-piperidine]-5-carboxylic acid;
1′-((4-chlorophenyl)carbamoyl)-2-oxospiro[indoline-3,4′-piperidine]-5-carboxylic acid;
2-oxo-1′-(4-(trifluoromethyl)benzoyl)spiro[indoline-3,4′-piperidine]-5-carboxylic acid;
1′-(2-(4-chlorophenoxy)acetyl)-2-oxospiro[indoline-3,4′-piperidine]-5-carboxylic acid;
1′-(cyclopropanecarbonyl)-2-oxospiro[indoline-3,4′-piperidine]-5-carboxylic acid;
1′-(4-chlorobenzoyl)-1-methyl-2-oxospiro[indoline-3,4′-piperidine]-5-carboxylic acid;
1′-acetyl-2-oxospiro[indoline-3,4′-piperidine]-5-carboxylic acid;
1′-isobutyryl-2-oxospiro[indoline-3,4′-piperidine]-5-carboxylic acid;
1′-(2-chlorobenzoyl)-2-oxospiro[indolineho-3,4′-piperidine]-5-carboxylic acid;
1′-(2-chlorobenzyl)-2-oxospiro[indoline-3,4′-piperidine]-5-carboxylic acid;
1′-(2,4-dichlorobenzyl)-2-oxospiro[indoline-3,4′-piperidine]-5-carboxylic acid;
1′-(2,4-dichlorobenzoyl)-2-oxospiro[indoline-3,4′-piperidine]-5-carboxylic acid;
1′-((3,5-dichloropyridin-2-yl)methyl)-2-oxospiro[indoline-3,4′-piperidine]-5-carboxylic acid;
   and stereoisomers and pharmaceutically acceptable salts thereof.

In some embodiments, the presently disclosed subject matter provides a compound of Formula (IB):

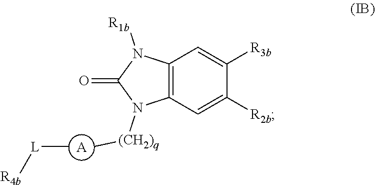

wherein:
q is 0 or 1;
A can be present or absent and when present is a 4-, 5-, or 6-membered cycloalkyl or cycloheteroalkyl ring;
R_{1b} is selected from the group consisting of H and substituted or unsubstituted branched or straightchain C₁-C₄ alkyl;
R_{2b} is H or —C(═O)—OR_{5b};
R_{3b} is selected from the group consisting of H, halogen, and —C(═O)—OR_{5b}, wherein R_{5b} is selected from the group consisting of H and substituted or unsubstituted branched or straightchain C₁-C₄ alkyl, provided that at least one of R_{1b} and R_{5b} is H;
R_{4b} is selected from the group consisting of substituted or unsubstituted branched or straightchain C₁-C₄ alkyl, substituted or unsubstituted cycloalkyl, substituted or unsubstituted cycloheteroalkyl, substituted or unsubstituted aryl, and substituted or unsubstituted heteroaryl;
L_{b} is selected from the group consisting of —C(═O)—, —(CH₂)ᵣ—, and —(CH₂)ₛ—O—; wherein r is selected from the group consisting of 1, 2, 3, or 4 and s is an integer selected from the group consisting of 0, 1, 2, or 3; and
stereoisomers and pharmaceutically acceptable salts thereof.

In certain embodiments, the compound of Formula (IB) is:

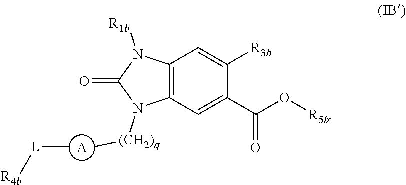

In some embodiments, R_{1b} and R_{5b} are each H.

In some embodiments, R_{3b} is H or F.

In some embodiments, A is present and is selected from the group consisting of azetidinyl, pyrrolidinyl, piperidinyl, and cyclohexyl:

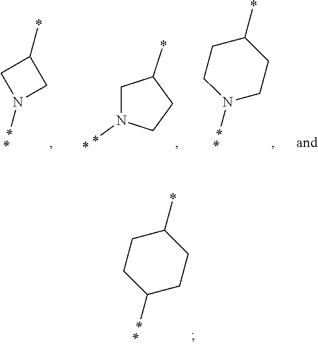

wherein * indicates a point of attachment of ring A to a nitrogen atom in the 3-position of the 2,3-dihydro-1H-benzo[d]imidazole ring and ** indicates a point of attachment of ring A to linker L_{b}.

In some embodiments, R₄ₐ is selected from the group consisting of methyl, isopropyl, cyclopropyl, substituted or unsubstituted phenyl, substituted or unsubstituted pyridyl, and substituted or unsubstituted pyrimidinyl.

In certain embodiments, the phenyl, pyridinyl, or pyrimidinyl is substituted with one or more substituent groups selected from the group consisting of substituted or unsubstituted branched or straightchain C₁-C₄ alkyl, C₁-C₄ alkoxyl, cyclopropyl, trifluoromethyl, hydroxyl, halogen, cyano, carbamoyl, and benzyloxy.

In particular embodiments, R_{4b} is halogen-substituted phenyl or halogen-substituted pyridyl.

In more particular embodiments, R_{4b} is selected from the group consisting of 2-chlorophenyl, 4-chlorophenyl, 2,4-dichlorophenyl, and 3,5-dichloropyridin-2-yl.

In yet more particular embodiments, the compound of Formula (IB) is selected from the group consisting of:
3-(1-(2-chlorobenzoyl)piperidin-4-yl)-2-oxo-2,3-dihydro-1H-benzo[d]imidazole-5-carboxylic acid;
3-(1-(2-chlorobenzyl)piperidin-4-yl)-2-oxo-2,3-dihydro-1H-benzo[d]imidazole-5-carboxylic acid;
3-(1-(2,4-dichlorobenzoyl)piperidin-4-yl)-2-oxo-2,3-dihydro-1H-benzo[d]imidazole-5-carboxylic acid;
3-((1r,4r)-4-((3,5-dichloropyridin-2-yl)oxy)cyclohexyl)-2-oxo-2,3-dihydro-1H-benzo[d]imidazole-5-carboxylic acid;
3-(1-(2,4-dichlorobenzyl)piperidin-4-yl)-2-oxo-2,3-dihydro-1H-benzo[d]imidazole-5-carboxylic acid;
3-((1s,4s)-4-((3,5-dichloropyridin-2-yl)oxy)cyclohexyl)-2-oxo-2,3-dihydro-1H-benzo[d]imidazole-5-carboxylic acid;
3-(2-(4-chlorophenoxy)ethyl)-2-oxo-2,3-dihydro-1H-benzo[d]imidazole-5-carboxylic acid;
3-(1-(2,4-dichlorobenzoyl)azetidin-3-yl)-2-oxo-2,3-dihydro-1H-benzo[d]imidazole-5-carboxylic acid;
3-(1-(2,4-dichlorobenzyl)azetidin-3-yl)-2-oxo-2,3-dihydro-1H-benzo[d]imidazole-5-carboxylic acid;
(S)-3-(1-(2,4-dichlorobenzoyl)pyrrolidin-3-yl)-2-oxo-2,3-dihydro-1H-benzo[d]imidazole-5-carboxylic acid;
(R)-3-(1-(2,4-dichlorobenzoyl)pyrrolidin-3-yl)-2-oxo-2,3-dihydro-1H-benzo[d]imidazole-5-carboxylic acid;
1-(1-(2,4-dichlorobenzoyl)piperidin-4-yl)-2-oxo-2,3-dihydro-1H-benzo[d]imidazole-5-carboxylic acid;
(S)-3-(1-(2,4-dichlorobenzyl)pyrrolidin-3-yl)-2-oxo-2,3-dihydro-1H-benzo[d]imidazole-5-carboxylic acid;
(R)-3-(1-(2,4-dichlorobenzyl)pyrrolidin-3-yl)-2-oxo-2,3-dihydro-1H-benzo[d]imidazole-5-carboxylic acid;
1-(1-(2,4-dichlorobenzyl)piperidin-4-yl)-2-oxo-2,3-dihydro-1H-benzo[d]imidazole-5-carboxylic acid;
3-(3-((3,5-dichloropyridin-2-yl)oxy)propyl)-2-oxo-2,3-dihydro-1H-benzo[d]imidazole-5-carboxylic acid;
3-((1-(2,4-dichlorobenzyl)pyrrolidin-3-yl)methyl)-2-oxo-2,3-dihydro-1H-benzo[d]imidazole-5-carboxylic acid;
3-((1-(2,4-dichlorobenzoyl)pyrrolidin-3-yl)methyl)-2-oxo-2,3-dihydro-1H-benzo[d]imidazole-5-carboxylic acid;
3-((1r,4r)-4-((3,5-dichloropyridin-2-yl)oxy)cyclohexyl)-6-fluoro-2-oxo-2,3-dihydro-1H-benzo[d]imidazole-5-carboxylic acid; and
   stereoisomers and pharmaceutically acceptable salts thereof.
      B. Methods for Treating a Condition, Disease, or Disorder Associated with IP6K

In some embodiments, the presently disclosed subject matter provides a method for treating a disease, condition, or disorder associated with IP6K, the method comprising administering a compound of Formula (IA) or Formula (IB), or a pharmaceutically acceptable salt thereof, to a subject in need of such treatment. In some embodiments, the disease, condition, or disorder is selected from the group consisting of a psychiatric disease, Alzheimer's disease, and diabetes. In particular embodiments, the psychiatric disease is bipolar disorder. In certain embodiments, the method of treating further comprises one or more of inhibiting IP6K, increasing AKT activity, and inhibiting GSK3 activity.

IP6K1 knock-out (KO) mice have altered behavioral phenotypes and impaired social interactions, in part due to IP6K1's effects on the AKT-GSK3 pathway (Chakraborty et al., 2014). GSK3 has been predominantly studied in the context of energy homeostasis and glycogen metabolism, but is also implicated in numerous disease states, including psychiatric diseases (Beurel et al., 2015; Beaulieu et al., 2012; Tan et al., 2012).

IP6K1 activates GSK3 via production of 5PP-IP5 which inhibits AKT's ability to phosphorylate and inactivate GSK3 (Chakraborty et al., 2010). Analysis of the postmortem schizophrenic brain revealed diminished levels of both AKT and phosphorylated (inactive) GSK3 (Emamian et al., 2004). Direct GSK3 inhibitors have been aggressively pursued for a number of years, yet their toxicity has limited clinical use (Bhat et al., 2018). Inhibiting GSK3 through the involvement of AKT and IP6K1 provides an improved strategy for careful regulation of this pathway.

The exact mechanism of the mood stabilizer lithium has remained elusive, however, a number of studies highlight the AKT/GSK3 pathway as an important target of the ion (Jope, 2003; Beaulieu et al., 2008). Nearly half the patients taking lithium for bipolar disorder to not respond, (Bowden, 2000), and those that do experience dose-limiting renal toxicity (Le Roy et al., 2009). One hypothesis for these divergent responses is due to differences in AKT activity (Pan et al. 2011). It has been noted that different strains of mice respond differently to lithium treatment, (Gould et al., 2007), and DBA/2J mice that do not respond to lithium were sensitized to the drug after expressing a constitutively active form of AKT. This effect was reversed after treatment with an AKT inhibitor (Pan et al. 2011). Thus, in mice, the therapeutic effects of lithium require robust AKT signaling activity. Thus, an IP6K inhibitor that increases Akt activity and decreases GSK3 activity may increase the amount of people that respond to lithium or allow those that do to take a lower efficacious dose and limit renal toxicity.

Type 2 diabetes (T2D) is a worldwide epidemic and a leading cause of cardiovascular events, renal diseases, non-traumatic loss of limb, and blindness. Many currently available drugs target different mechanisms, such as incretin regulation, insulin resistance, glucose reabsorption, and dopamine signaling (Miller et al., 2019). These drugs have different efficacy and adverse effect profiles, such as hypoglycemia, weight gain, renal function limitations, and gastrointestinal symptoms. Therefore, additional targeted therapies with complementary mechanisms are needed to improve management of T2D in patients where current drugs have moderate efficacy or contraindicative effects.

Under physiological conditions, insulin activates the tyrosine kinase of the insulin receptor (INSR), which stimulates insulin receptor substrate (IRS) phosphorylation followed by activation of phosphatidylinositol-4,5-bisphosphate-3 kinase (PI3K) and AKT (Petersen and Shulman, 2018). Insulin resistance is a major pathological defect in T2D patients. Insulin sensitizers, such as peroxisome proliferator-activated receptor gamma (PPARγ) activating thiazolidinediones (TZDs), including rosiglitazone and pioglitazone, have been approved for the treatment of T2D. Pioglitazone and rosiglitazone, however, had marketing authorization withdrawn under the EMA and the FDA has required a black box warning for all TZDs due to an increased risk in cardiovascular events in 2007 (Hickson et al., 2019). Additionally, there is evidence that pioglitazone may increase bladder cancer risk (Marks, 2013). Although the black box warning for rosiglitazone was removed by the FDA in 2013, prescription sales declined sharply for both drugs (Hickson et al., 2019). Therefore, there is an unmet medical need for new strategies to improve insulin sensitivity.

Skeletal, hepatic and adipose insulin resistance are all traceable to impairments at the most proximal levels of insulin signaling: INSR, IRS1, PI3K, and AKT activity (Petersen and Shulman, 2018). Consistent with this, experimental methods that increase AKT signaling have been shown to improve insulin sensitivity in various animal and cellular models of T2D (Petersen and Shulman, 2018). Therapeutic strategies of modest, indirect modulation of the AKT pathway to compensate for the decrease of AKT activity due to insulin resistance will be a safe and effective approach to improve insulin sensitivity and provide an additional option to improve T2D treatment.

AKT resides in the cytosol in an inactive conformation. Recruitment of AKT by the second messenger PI(3,4,5)P₃ (PIP3) to the plasma membrane induces conformational changes in the structure of AKT and exposes phosphorylation sites T308 in the kinase domain and S473 in the C-terminal domain. AKT is partially activated by phosphorylation of T308 by PDK1. Full activation requires phosphorylation of S473, which can be catalyzed by members of the PI3K-related kinase family mTORC2 or DNA-PK (Fayard et al., 2010). In competition with PIP3, inositol pyrophosphates, such as diphosphoinositol pentakisphosphate (IP7), also can bind to AKT and inhibit the PIP3 induced phosphorylation at T308, (Chakraborty et al., 2010), thereby serving as a negative regulator of AKT activation. IP7 is formed from inositol hexakisphosphate (IP6) by a family of three IP6 kinases (IP6Ks) named IP6K1, IP6K2 and IP6K3. In hepatocytes of IP6K1 knockout mice, the IP7 level decreases more than 60%, suggesting that IP6K1 is the major isoform responsible for IP7 production in liver. IP6K1 deletion results in a significant increase in pT308-AKT and pS9-GSK3 in vivo (Chakraborty et al., 2010). By inhibiting IP6K1 and reducing cellular IP7 concentrations, this negative regulatory effect on AKT will be reduced, resulting in a potentiation of AKT signaling that improves insulin sensitivity.

Extensive studies using cellular or in vivo IPK1 genetic knockout, as well as pharmacological inhibition with the known pan-IP6K inhibitor N2-(m-trifluorobenzyl)-N6-(p-nitrobenzyl)purine (TNP), have provided compelling evidence that inhibiting the IP6K1 enzyme will produce beneficial effects in T2D. Consistent with its modulatory effect on the AKT and insulin signaling pathway, IP6K1 knockout mice displayed increased insulin sensitivity (Chakraborty et al., 2010). The mice do not display high fat diet (HFD) induced impaired glucose tolerance, insulin resistance or hyperglycemia. In addition, IP6K1 knockout mice are resistant to HFD-induced obesity, which is not due to a change in food intake, but more likely results from the resistance to HDF-induced reductions in oxygen consumption and carbon dioxide release (Chakraborty et al., 2010). Consistent with this, pharmacologic inhibition of IP6K with the IP6K inhibitor TNP displayed a strong anti-obesity and anti-diabetic effect (Ghoshal et al., 2016). Mice treated with HFD for 8 weeks followed by daily intraperitoneal injection of 20 mg/kg TNP and HFD for 18 days had lower serum insulin levels while displaying improved glucose tolerance and insulin sensitivity, suggesting that IP6K1 inhibition by TNP increases insulin sensitivity (Ghoshal et al., 2016). TNP, however, lacks overall drug-like properties and new inhibitors are needed to fully capitalize on this treatment strategy. In human clinical studies, muscle IP6K1 protein content is elevated after lean meat ingestion in obese adults, but not in lean individuals. Therefore, dysregulation of IP6K1 in obese adults may contribute to the development of insulin resistance, (Barclay et al., 2019), further supporting that inhibition of IP6K may improve insulin sensitivity in T2D patients.

AMP-activated protein kinase (AMPK) is a central regulator for cellular energy homeostasis by sensing the cellular ATP level. IP6K1 also is found to interact with AMPK, and deletion of IP6K1 increases AMPK activity (Zhu et al., 2016). A decrease in cellular ATP results in an increase in ADP and AMP levels, leading to the activation of AMPK. AMPK activation promotes catabolism for production of ATP and inhibits the consumption of ATP—effectively stimulating glycolysis and coupled respiration-mediated ATP generation to restore optimal ATP concentrations. Several pharmacological activators for AMPK, including PF-793, MK-8722 and 0304, have all been shown to increase glucose uptake in an insulin-independent manner (Cokorinos et al., 2017; Myers et al., 2017; Steneberg et al., 2018). Therefore, inhibition of IP6K1 also may exert its anti-diabetic effect through the activation of AMPK.

IP7 can be further phosphorylated by PPIP5K to form 1,5-bisdiphosphoinositol tetrakisphosphate (IP8). Both IP7 and IP8 have high-energy pyrophosphate groups, which have been proposed to work in concert for sensing and controlling cellular ATP levels (Gu et al., 2017). In comparison to their unmodified counterparts, IP6K1 knockout (which reduces both IP7 and IP8) in a variety of cell types such as S. cerevisiae, mouse embryonic fibroblasts (MEF), adipocytes, cardiomyocytes, HEK293, and HCT116 cells all show increased ATP levels (Zhu et al., 2016; Cokorinos et al., 2017). This has been proposed to be through an increase in glycolytic and/or mitochondrial activity which appears to be sensitive to IP7 and IP8 levels (Gu et al., 2017; Szijgyarto et al., 2011; Sun et al., 2015).

Functionally, the change in ATP has been linked to increased mitochondrial respiration in IP6K1 knockout MEF and HCT116 cells as well as in cardiomyocytes (Myers et al., 2017). In T2D both basal and insulin-stimulated ATP synthesis in liver and skeletal muscle are impaired, (Szendroedi et al., 2007; Koliaki and Roden, 2013), likely resulting from decreased mitochondrial activity and decreased total content of skeletal mitochondria (Petersen and Shulman, 2018). Therefore, inhibition of IP6K1 may improve the cellular mitochondrial activity and restore proper ATP levels in T2D patients, providing another mechanism for benefit in the treatment for diabetes.

As used herein, the term “inhibit,” and grammatical derivations thereof, refers to the ability of a presently disclosed compound, e.g., a presently disclosed compound of Formula (IA) or Formula (IB), to block, partially block, interfere, decrease, or reduce the activity or expression of IP6K in a subject. Thus, one of ordinary skill in the art would appreciate that the term “inhibit” encompasses a complete and/or partial decrease in the function of the channel, e.g., a decrease by at least 10%, in some embodiments, a decrease by at least 20%, 30%, 50%, 75%, 95%, 98%, and up to and including 100%.

In particular embodiments, the presently disclosed subject matter provides a method for treating a condition, disease, or disorder associated with an increased IP6K activity or expression.

The presently disclosed subject matter also includes use of a compound of Formula (IA) or Formula (IB) in the manufacture of a medicament for treating a condition, disease, or disorder associated with an increased IP6K activity or expression in a subject afflicted with such a disorder.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the makeup of the pharmaceutical composition, the target tissue, and the like.

The term “combination” is used in its broadest sense and means that a subject is administered at least two agents, more particularly a compound of Formula (IA) or Formula (IB) and at least one analgesic; and, optionally, one or more analgesic agents. More particularly, the term “in combination” refers to the concomitant administration of two (or more) active agents for the treatment of a, e.g., single disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one but not the other). The single dosage form may include additional active agents for the treatment of the disease state.

Further, the compounds of Formula (IA) or Formula (IB) described herein can be administered alone or in combination with adjuvants that enhance stability of the compounds of Formula (IA) or Formula (IB), alone or in combination with one or more analgesic agents, facilitate administration of pharmaceutical compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of a compound of Formula (IA) or Formula (IB) and at least one additional therapeutic agent can be varied so long as the beneficial effects of the combination of these agents are achieved. Accordingly, the phrase “in combination with” refers to the administration of a compound of Formula (IA) or Formula (IB) and at least one additional therapeutic agent either simultaneously, sequentially, or a combination thereof. Therefore, a subject administered a combination of a compound of Formula (IA) or Formula (IB) and at least one additional therapeutic agent can receive compound of Formula (IA) or Formula (IB) and at least one additional therapeutic agent at the same time (i.e., simultaneously) or at different times (i.e., sequentially, in either order, on the same day or on different days), so long as the effect of the combination of both agents is achieved in the subject.

When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the compound of Formula (IA) or Formula (IB) and at least one additional therapeutic agent are administered simultaneously, they can be administered to the subject as separate pharmaceutical compositions, each comprising either a compound of Formula (IA) or Formula (IB) or at least one additional therapeutic agent, or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of a compound of Formula (IA) or Formula (IB) and at least one additional therapeutic agent is greater than the sum of the biological activities of the respective agents when administered individually.

Synergy can be expressed in terms of a “Synergy Index (SI),” which generally can be determined by the method described by F. C. Kull et al., Applied Microbiology 9, 538 (1961), from the ratio determined by:

$${Q}_{a} / {Q}_{A} + {Q}_{b} / {Q}_{B} = Synergy Index ( S I )$$

wherein:
Q_{A} is the concentration of a component A, acting alone, which produced an end point in relation to component A;
Qₐ is the concentration of component A, in a mixture, which produced an end point;
Q_{B} is the concentration of a component B, acting alone, which produced an end point in relation to component B; and
Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

### C. Pharmaceutical Compositions and Administration

In another aspect, the present disclosure provides a pharmaceutical composition including one compound of Formula (IA) or Formula (IB) alone or in combination with one or more additional therapeutic agents in admixture with a pharmaceutically acceptable excipient. One of ordinary skill in the art will recognize that the pharmaceutical compositions include the pharmaceutically acceptable salts of the compounds described above. Pharmaceutically acceptable salts are generally well known to those of ordinary skill in the art and include salts of active compounds which are prepared with relatively nontoxic acids or bases, depending on the particular substituent moieties found on the compounds described herein. When compounds of the present disclosure contain relatively acidic functionalities, base addition salts can be obtained by contacting the neutral form of such compounds with a sufficient amount of the desired base, either neat or in a suitable inert solvent or by ion exchange, whereby one basic counterion (base) in an ionic complex is substituted for another. Examples of pharmaceutically acceptable base addition salts include sodium, potassium, calcium, zinc, magnesium, ammonium, piperidine, piperazine, organic amino, or a similar salt.

When compounds of the present disclosure contain relatively basic functionalities, acid addition salts can be obtained by contacting the neutral form of such compounds with a sufficient amount of the desired acid, either neat or in a suitable inert solvent or by ion exchange, whereby one acidic counterion (acid) in an ionic complex is substituted for another. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-toluenesulfonic, citric, tartaric, methanesulfonic, and the like. Also included are salts of amino acids such as arginate and the like, and salts of organic acids like glucuronic or galactunoric acids and the like (see, for example, Berge et al, “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Accordingly, pharmaceutically acceptable salts suitable for use with the presently disclosed subject matter include, by way of example but not limitation, acetate, benzenesulfonate, benzoate, bicarbonate, bitartrate, bromide, calcium edetate, camsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington: The Science and Practice of Pharmacy (20^{th} ed.) Lippincott, Williams & Wilkins (2000).

In therapeutic and/or diagnostic applications, the compounds of the disclosure can be formulated for a variety of modes of administration, including systemic and topical or localized administration. Techniques and formulations generally may be found in Remington: The Science and Practice of Pharmacy (20^{th} ed.) Lippincott, Williams & Wilkins (2000).

Depending on the specific conditions being treated, such agents may be formulated into liquid or solid dosage forms and administered systemically or locally. The agents may be delivered, for example, in a timed- or sustained-slow release form as is known to those skilled in the art. Techniques for formulation and administration may be found in Remington: The Science and Practice of Pharmacy (20^{th} ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra-sternal, intra-synovial, intra-hepatic, intralesional, intracranial, intraperitoneal, intranasal, or intraocular injections or other modes of delivery.

For injection, the agents of the disclosure may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, the compositions of the present disclosure, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions and the like, for oral ingestion by a subject (e.g., patient) to be treated.

For nasal or inhalation delivery, the agents of the disclosure also may be formulated by methods known to those of ordinary skill in the art, and may include, for example, but not limited to, examples of solubilizing, diluting, or dispersing substances, such as saline; preservatives, such as benzyl alcohol; absorption promoters; and fluorocarbons.

Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein. Generally, the compounds according to the disclosure are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. A non-limiting dosage is 10 to 30 mg per day. The exact dosage will depend upon the route of administration, the form in which the compound is administered, the subject to be treated, the body weight of the subject to be treated, the bioavailability of the compound(s), the adsorption, distribution, metabolism, and excretion (ADME) toxicity of the compound(s), and the preference and experience of the attending physician.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

### II. Chemical Definitions

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

While the following terms in relation to compounds of Formula (IA) or Formula (IB) are believed to be well understood by one of ordinary skill in the art, the following definitions are set forth to facilitate explanation of the presently disclosed subject matter. These definitions are intended to supplement and illustrate, not preclude, the definitions that would be apparent to one of ordinary skill in the art upon review of the present disclosure.

The terms substituted, whether preceded by the term “optionally” or not, and substituent, as used herein, refer to the ability, as appreciated by one skilled in this art, to change one functional group for another functional group on a molecule, provided that the valency of all atoms is maintained. When more than one position in any given structure may be substituted with more than one substituent selected from a specified group, the substituent may be either the same or different at every position.

The substituents also may be further substituted (e.g., an aryl group substituent may have another substituent off it, such as another aryl group, which is further substituted at one or more positions).

Where substituent groups or linking groups are specified by their conventional chemical formulae, written from left to right, they equally encompass the chemically identical substituents that would result from writing the structure from right to left, e.g., —CH₂O— is equivalent to —OCH₂—; —C(═O)O— is equivalent to —OC(═O)—; —OC(═O)NR— is equivalent to —NRC(═O)O—, and the like.

When the term “independently selected” is used, the substituents being referred to (e.g., R groups, such as groups R₁, R₂, and the like, or variables, such as “m” and “n”), can be identical or different. For example, both R₁ and R₂ can be substituted alkyls, or R₁ can be hydrogen and R₂ can be a substituted alkyl, and the like.

The terms “a,” “an,” or “a(n),” when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R-substituted.” Where a moiety is R-substituted, the moiety is substituted with at least one R substituent and each R substituent is optionally different.

A named “R” or group will generally have the structure that is recognized in the art as corresponding to a group having that name, unless specified otherwise herein. For the purposes of illustration, certain representative “R” groups as set forth above are defined below.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

Unless otherwise explicitly defined, a “substituent group,” as used herein, includes a functional group selected from one or more of the following moieties, which are defined herein:

The term hydrocarbon, as used herein, refers to any chemical group comprising hydrogen and carbon. The hydrocarbon may be substituted or unsubstituted. As would be known to one skilled in this art, all valencies must be satisfied in making any substitutions. The hydrocarbon may be unsaturated, saturated, branched, unbranched, cyclic, polycyclic, or heterocyclic. Illustrative hydrocarbons are further defined herein below and include, for example, methyl, ethyl, n-propyl, isopropyl, cyclopropyl, allyl, vinyl, n-butyl, tert-butyl, ethynyl, cyclohexyl, and the like.

The term “alkyl,” by itself or as part of another substituent, means, unless otherwise stated, a straight (i.e., unbranched) or branched chain, acyclic or cyclic hydrocarbon group, or combination thereof, which may be fully saturated, mono- or polyunsaturated and can include di- and multivalent groups, having the number of carbon atoms designated (i.e., C₁₋₁₀ means one to ten carbons, including 1, 2, 3, 4, 5, 6, 7, 8, 9, and 10 carbons). In particular embodiments, the term “alkyl” refers to C₁₋₂₀ inclusive, including 1, 2, 3, 4, 5, 6, 7, 8, 9, 10, 11, 12, 13, 14, 15, 16, 17, 18, 19, and 20 carbons, linear (i.e., “straight-chain”), branched, or cyclic, saturated or at least partially and in some cases fully unsaturated (i.e., alkenyl and alkynyl) hydrocarbon radicals derived from a hydrocarbon moiety containing between one and twenty carbon atoms by removal of a single hydrogen atom.

C₁-C₄ alkyl includes C₁, C₂, C₃, and C₄ alkyl, including methyl, ethyl, propyl, isopropyl, butyl, isobutyl, sec-butyl, and t-butyl.

Representative saturated hydrocarbon groups include, but are not limited to, methyl, ethyl, n-propyl, isopropyl, n-butyl, isobutyl, sec-butyl, tert-butyl, n-pentyl, sec-pentyl, isopentyl, neopentyl, n-hexyl, sec-hexyl, n-heptyl, n-octyl, n-decyl, n-undecyl, dodecyl, cyclohexyl, (cyclohexyl)methyl, cyclopropylmethyl, and homologs and isomers thereof.

“Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, cyano, and mercapto.

The term “heteroalkyl,” by itself or in combination with another term, means, unless otherwise stated, a stable straight or branched chain having from 1 to 20 carbon atoms or heteroatoms or a cyclic hydrocarbon group having from 3 to 10 carbon atoms or heteroatoms, or combinations thereof, consisting of at least one carbon atom and at least one heteroatom selected from the group consisting of O, N, P, Si and S, and wherein the nitrogen, phosphorus, and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, O—CH₃, —O—CH₂—CH₃, and —CN. Up to two or three heteroatoms may be consecutive, such as, for example, —CH₂—NH—OCH₃ and —CH₂—O—Si(CH₃)₃.

As described above, heteroalkyl groups, as used herein, include those groups that are attached to the remainder of the molecule through a heteroatom, such as —C(O)NR′, —NR′R″, —OR′, —SR, —S(O)R, and/or —S(O₂)R′. Where “heteroalkyl” is recited, followed by recitations of specific heteroalkyl groups, such as —NR′R or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, unsubstituted alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl, and fused ring systems, such as dihydro- and tetrahydronaphthalene, and the like.

The term “cycloalkylalkyl,” as used herein, refers to a cycloalkyl group as defined hereinabove, which is attached to the parent molecular moiety through an alkylene moiety, also as defined above, e.g., a C₁₋₂₀ alkylene moiety. Examples of cycloalkylalkyl groups include cyclopropylmethyl and cyclopentylethyl.

The terms “cycloheteroalkyl” or “heterocycloalkyl” refer to a non-aromatic ring system, unsaturated or partially unsaturated ring system, such as a 3- to 10-member substituted or unsubstituted cycloalkyl ring system, including one or more heteroatoms, which can be the same or different, and are selected from the group consisting of nitrogen (N), oxygen (O), sulfur (S), phosphorus (P), and silicon (Si), and optionally can include one or more double bonds.

The cycloheteroalkyl ring can be optionally fused to or otherwise attached to other cycloheteroalkyl rings and/or non-aromatic hydrocarbon rings. Heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocylic refers to a non-aromatic 5-, 6-, or 7-membered ring or a polycyclic group wherein at least one ring atom is a heteroatom selected from O, S, and N (wherein the nitrogen and sulfur heteroatoms may be optionally oxidized), including, but not limited to, a bi- or tri-cyclic group, comprising fused six-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring. Representative cycloheteroalkyl ring systems include, but are not limited to pyrrolidinyl, pyrrolinyl, imidazolidinyl, imidazolinyl, pyrazolidinyl, pyrazolinyl, piperidinyl, piperazinyl, indolinyl, quinuclidinyl, morpholinyl, thiomorpholinyl, thiadiazinanyl, tetrahydrofuranyl, and the like.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The terms “cycloalkylene” and “heterocycloalkylene” refer to the divalent derivatives of cycloalkyl and heterocycloalkyl, respectively.

An unsaturated hydrocarbon has one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups which are limited to hydrocarbon groups are termed “homoalkyl.”

More particularly, the term “alkenyl” as used herein refers to a monovalent group derived from a C₂₋₂₀ inclusive straight or branched hydrocarbon moiety having at least one carbon-carbon double bond by the removal of a single hydrogen molecule. Alkenyl groups include, for example, ethenyl (i.e., vinyl), propenyl, butenyl, 1-methyl-2-buten-1-yl, pentenyl, hexenyl, octenyl, allenyl, and butadienyl.

The term “cycloalkenyl” as used herein refers to a cyclic hydrocarbon containing at least one carbon-carbon double bond. Examples of cycloalkenyl groups include cyclopropenyl, cyclobutenyl, cyclopentenyl, cyclopentadiene, cyclohexenyl, 1,3-cyclohexadiene, cycloheptenyl, cycloheptatrienyl, and cyclooctenyl.

The term “alkynyl” as used herein refers to a monovalent group derived from a straight or branched C₂₋₂₀ hydrocarbon of a designed number of carbon atoms containing at least one carbon-carbon triple bond. Examples of “alkynyl” include ethynyl, 2-propynyl (propargyl), 1-propynyl, pentynyl, hexynyl, and heptynyl groups, and the like.

The term “alkylene” by itself or a part of another substituent refers to a straight or branched bivalent aliphatic hydrocarbon group derived from an alkyl group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —CH₂CH₂CH₂CH₂—, —CH₂CH═CHCH₂—, —CH₂CsCCH₂—, —CH₂CH₂CH(CH₂CH₂CH₃)CH₂—, —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons. Typically, an alkyl (or alkylene) group will have from 1 to 24 carbon atoms, with those groups having 10 or fewer carbon atoms being some embodiments of the present disclosure. A “lower alkyl” or “lower alkylene” is a shorter chain alkyl or alkylene group, generally having eight or fewer carbon atoms.

The term “heteroalkylene” by itself or as part of another substituent means a divalent group derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms also can occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′— represents both —C(O)OR′— and —R′OC(O)—.

The term “aryl” means, unless otherwise stated, an aromatic hydrocarbon substituent that can be a single ring or multiple rings (such as from 1 to 3 rings), which are fused together or linked covalently. The term “heteroaryl” refers to aryl groups (or rings) that contain from one to four heteroatoms (in each separate ring in the case of multiple rings) selected from N, O, and S, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of above noted aryl and heteroaryl ring systems are selected from the group of acceptable substituents described below. The terms “arylene” and “heteroarylene” refer to the divalent forms of aryl and heteroaryl, respectively.

For brevity, the term “aryl” when used in combination with other terms (e.g., aryloxy, arylthioxy, arylalkyl) includes both aryl and heteroaryl rings as defined above. Thus, the terms “arylalkyl” and “heteroarylalkyl” are meant to include those groups in which an aryl or heteroaryl group is attached to an alkyl group (e.g., benzyl, phenethyl, pyridylmethyl, furylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like). However, the term “haloaryl,” as used herein is meant to cover only aryls substituted with one or more halogens.

Where a heteroalkyl, heterocycloalkyl, or heteroaryl includes a specific number of members (e.g. “3 to 7 membered”), the term “member” refers to a carbon or heteroatom.

Further, a structure represented generally by the formula:

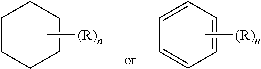

as used herein refers to a ring structure, for example, but not limited to a 3-carbon, a 4-carbon, a 5-carbon, a 6-carbon, a 7-carbon, and the like, aliphatic and/or aromatic cyclic compound, including a saturated ring structure, a partially saturated ring structure, and an unsaturated ring structure, comprising a substituent R group, wherein the R group can be present or absent, and when present, one or more R groups can each be substituted on one or more available carbon atoms of the ring structure. The presence or absence of the R group and number of R groups is determined by the value of the variable “n,” which is an integer generally having a value ranging from 0 to the number of carbon atoms on the ring available for substitution. Each R group, if more than one, is substituted on an available carbon of the ring structure rather than on another R group. For example, the structure above where n is 0 to 2 would comprise compound groups including, but not limited to:

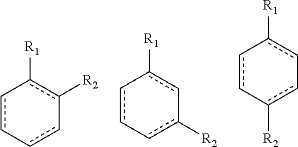

and the like.

A dashed line, e.g., [symbol] , representing a bond in a cyclic ring structure indicates that the bond can be either present or absent in the ring. That is, a dashed line representing a bond in a cyclic ring structure indicates that the ring structure is selected from the group consisting of a saturated ring structure, a partially saturated ring structure, and an unsaturated ring structure.

The symbol ( [symbol] ) denotes the point of attachment of a moiety to the remainder of the molecule.

When a named atom of an aromatic ring or a heterocyclic aromatic ring is defined as being “absent,” the named atom is replaced by a direct bond.

Each of above terms (e.g., “alkyl,” “heteroalkyl,” “cycloalkyl, and “heterocycloalkyl”, “aryl,” “heteroaryl,” “phosphonate,” and “sulfonate” as well as their divalent derivatives) are meant to include both substituted and unsubstituted forms of the indicated group. Optional substituents for each type of group are provided below.

Substituents for alkyl, heteroalkyl, cycloalkyl, heterocycloalkyl monovalent and divalent derivative groups (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, =N—OR′, —NR′R″, —SR′, -halogen, —SiR′R″R″′, —OC(O)R′, —C(O)R′, —CO₂R′, —C(O)NR′R″, —OC(O)NR′R″, —NR″C(O)R′, —NR′—C(O)NR″R″′, —NR″C(O)OR′, —NR—C(NR′R″)=NR″′, —S(O)R′, —S(O)₂R′, —S(O)₂NR′R″, —NRSO₂R′, —CN, CF₃, fluorinated C₁₋₄ alkyl, and —NO₂ in a number ranging from zero to (2m′+1), where m′ is the total number of carbon atoms in such groups. R′, R″, R″′ and R″″ each may independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. As used herein, an “alkoxy” group is an alkyl attached to the remainder of the molecule through a divalent oxygen. When a compound of the disclosure includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituents, one of ordinary skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for alkyl groups above, exemplary substituents for aryl and heteroaryl groups (as well as their divalent derivatives) are varied and are selected from, for example: halogen, —OR′, —NR′R″, —SR′, —SiR′R″R″′, —OC(O)R′, —C(O)R′, —CO₂R′, —C(O)NR′R″, —OC(O)NR′R″, —NR″C(O)R′, —NR′—C(O)NR″R″′, —NR″C(O)OR′, —NR—C(NR′R″R′″)=NR″″, —NR—C(NR′R″)=NR″′—S(O)R′, —S(O)₂R′, —S(O)₂NR′R″, —NRSO₂R′, —CN and —NO₂, —R′, —N₃, —CH(Ph)₂, fluoro(C₁₋₄)alkoxo, and fluoro(C₁₋₄)alkyl, in a number ranging from zero to the total number of open valences on aromatic ring system; and where R′, R″, R″′ and R″″ may be independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound of the disclosure includes more than one R group, for example, each of the R groups is independently selected as are each R′, R″, R″′ and R″″ groups when more than one of these groups is present.

Two of the substituents on adjacent atoms of aryl or heteroaryl ring may optionally form a ring of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4.

One of the single bonds of the new ring so formed may optionally be replaced with a double bond. Alternatively, two of the substituents on adjacent atoms of aryl or heteroaryl ring may optionally be replaced with a substituent of the formula —(CRR′)ₛ—X′—(C″R″′)_{d}—, where s and d are independently integers of from 0 to 3, and X′ is —O—, —NR′—, —S—, —S(O)—, —S(O)₂—, or —S(O)₂NR′—. The substituents R, R′, R″ and R″′ may be independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “acyl” refers to an organic acid group wherein the —OH of the carboxyl group has been replaced with another substituent and has the general formula RC(═O)—, wherein R is an alkyl, alkenyl, alkynyl, aryl, carbocylic, heterocyclic, or aromatic heterocyclic group as defined herein). As such, the term “acyl” specifically includes arylacyl groups, such as a 2-(furan-2-yl)acetyl)- and a 2-phenylacetyl group. Specific examples of acyl groups include acetyl and benzoyl. Acyl groups also are intended to include amides, —RC(═O)NR′, esters, —RC(═O)OR′, ketones, —RC(═O)R′, and aldehydes, —RC(═O)H.

The terms “alkoxyl” or “alkoxy” are used interchangeably herein and refer to a saturated (i.e., alkyl-O—) or unsaturated (i.e., alkenyl-O— and alkynyl-O—) group attached to the parent molecular moiety through an oxygen atom, wherein the terms “alkyl,” “alkenyl,” and “alkynyl” are as previously described and can include C₁₋₂₀ inclusive, linear, branched, or cyclic, saturated or unsaturated oxo-hydrocarbon chains, including, for example, methoxyl, ethoxyl, propoxyl, isopropoxyl, n-butoxyl, sec-butoxyl, tert-butoxyl, and n-pentoxyl, neopentoxyl, n-hexoxyl, and the like.

The term “alkoxyalkyl” as used herein refers to an alkyl-O-alkyl ether, for example, a methoxyethyl or an ethoxymethyl group.

“Aryloxyl” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl.

“Aralkyl” refers to an aryl-alkyl-group wherein aryl and alkyl are as previously described and include substituted aryl and substituted alkyl. Exemplary aralkyl groups include benzyl, phenylethyl, and naphthylmethyl.

“Aralkyloxyl” refers to an aralkyl-O—group wherein the aralkyl group is as previously described. An exemplary aralkyloxyl group is benzyloxyl, i.e., C₆H₅—CH₂—O—. An aralkyloxyl group can optionally be substituted.

“Alkoxycarbonyl” refers to an alkyl-O—C(═O)— group. Exemplary alkoxycarbonyl groups include methoxycarbonyl, ethoxycarbonyl, butyloxycarbonyl, and tert-butyloxycarbonyl.

“Aryloxycarbonyl” refers to an aryl-O—C(═O)— group. Exemplary aryloxycarbonyl groups include phenoxy- and naphthoxy-carbonyl.

“Aralkoxycarbonyl” refers to an aralkyl-O—C(═O)— group. An exemplary aralkoxycarbonyl group is benzyloxycarbonyl.

“Carbamoyl” refers to an amide group of the formula —C(═O)NH₂. “Alkylcarbamoyl” refers to a R′RN—C(═O)— group wherein one of R and R′ is hydrogen and the other of R and R′ is alkyl and/or substituted alkyl as previously described. “Dialkylcarbamoyl” refers to a R′RN—C(═O)— group wherein each of R and R′ is independently alkyl and/or substituted alkyl as previously described.

The term carbonyldioxyl, as used herein, refers to a carbonate group of the formula —O—C(═O)—OR.

“Acyloxyl” refers to an acyl-O—group wherein acyl is as previously described.

The term “amino” refers to the —NH₂ group and also refers to a nitrogen containing group as is known in the art derived from ammonia by the replacement of one or more hydrogen radicals by organic groups. For example, the terms “acylamino” and “alkylamino” refer to specific N-substituted organic groups with acyl and alkyl substituent groups respectively.

An “aminoalkyl” as used herein refers to an amino group covalently bound to an alkylene linker. More particularly, the terms alkylamino, dialkylamino, and trialkylamino as used herein refer to one, two, or three, respectively, alkyl groups, as previously defined, attached to the parent molecular moiety through a nitrogen atom. The term alkylamino refers to a group having the structure —NHR′ wherein R′ is an alkyl group, as previously defined; whereas the term dialkylamino refers to a group having the structure —NR′R″, wherein R′ and R″ are each independently selected from the group consisting of alkyl groups. The term trialkylamino refers to a group having the structure —NR′R″R″′, wherein R′, R″, and R′″ are each independently selected from the group consisting of alkyl groups. Additionally, R′, R″, and/or R″′ taken together may optionally be —(CH₂)ₖ— where k is an integer from 2 to 6. Examples include, but are not limited to, methylamino, dimethylamino, ethylamino, diethylamino, diethylaminocarbonyl, methylethylamino, isopropylamino, piperidino, trimethylamino, and propylamino.

The amino group is —NR′R″, wherein R′ and R″ are typically selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

The terms alkylthioether and thioalkoxyl refer to a saturated (i.e., alkyl-S—) or unsaturated (i.e., alkenyl-S—and alkynyl-S—) group attached to the parent molecular moiety through a sulfur atom. Examples of thioalkoxyl moieties include, but are not limited to, methylthio, ethylthio, propylthio, isopropylthio, n-butylthio, and the like.

“Acylamino” refers to an acyl-NH—group wherein acyl is as previously described. “Aroylamino” refers to an aroyl-NH—group wherein aroyl is as previously described.

The term “carbonyl” refers to the —C(═O)—group, and can include an aldehyde group represented by the general formula R—C(═O)H.

The term “carboxyl” refers to the —COOH group. Such groups also are referred to herein as a “carboxylic acid” moiety.

The term “cyano” refers to the —C≡N group.

The terms “halo,” “halide,” or “halogen” as used herein refer to fluoro, chloro, bromo, and iodo groups. Additionally, terms such as “haloalkyl,” are meant to include monohaloalkyl and polyhaloalkyl. For example, the term “halo(C₁₋₄)alkyl” is mean to include, but not be limited to, trifluoromethyl, 2,2,2-trifluoroethyl, 4-chlorobutyl, 3-bromopropyl, and the like.

The term “hydroxyl” refers to the —OH group.

The term “hydroxyalkyl” refers to an alkyl group substituted with an —OH group.

The term “mercapto” refers to the —SH group.

The term “oxo” as used herein means an oxygen atom that is double bonded to a carbon atom or to another element.

The term “nitro” refers to the —NO₂ group.

The term “thio” refers to a compound described previously herein wherein a carbon or oxygen atom is replaced by a sulfur atom.

The term “sulfate” refers to the —SO₄ group.

The term thiohydroxyl or thiol, as used herein, refers to a group of the formula —SH.

More particularly, the term “sulfide” refers to compound having a group of the formula —SR.

The term “sulfone” refers to compound having a sulfonyl group —S(O₂)R′.

The term “sulfoxide” refers to a compound having a sulfinyl group —S(O)R

The term ureido refers to a urea group of the formula —NH—CO—NH₂.

Throughout the specification and claims, a given chemical formula or name shall encompass all tautomers, congeners, and optical- and stereoisomers, as well as racemic mixtures where such isomers and mixtures exist.

Certain compounds of the present disclosure may possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as D- or L- for amino acids, and individual isomers are encompassed within the scope of the present disclosure. The compounds of the present disclosure do not include those which are known in art to be too unstable to synthesize and/or isolate. The present disclosure is meant to include compounds in racemic, scalemic, and optically pure forms. Optically active (R)- and (S)-, or D- and L-isomers may be prepared using chiral synthons or chiral reagents or resolved using conventional techniques. When the compounds described herein contain olefenic bonds or other centers of geometric asymmetry, and unless specified otherwise, it is intended that the compounds include both E and Z geometric isomers.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

It will be apparent to one skilled in the art that certain compounds of this disclosure may exist in tautomeric forms, all such tautomeric forms of the compounds being within the scope of the disclosure. The term “tautomer,” as used herein, refers to one of two or more structural isomers which exist in equilibrium and which are readily converted from one isomeric form to another.

Unless otherwise stated, structures depicted herein are also meant to include compounds which differ only in the presence of one or more isotopically enriched atoms. For example, compounds having the present structures with the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of atoms that constitute such compounds. For example, the compounds may be radiolabeled with radioactive isotopes, such as for example deuterium (²H), tritium (³H), iodine-125 (¹²⁵I) or carbon-14 (¹⁴C). All isotopic variations of the compounds of the present disclosure, whether radioactive or not, are encompassed within the scope of the present disclosure.

The compounds of the present disclosure may exist as salts. The present disclosure includes such salts. Examples of applicable salt forms include hydrochlorides, hydrobromides, sulfates, methanesulfonates, nitrates, maleates, acetates, citrates, fumarates, tartrates (e.g. (+)-tartrates, (−)-tartrates or mixtures thereof including racemic mixtures, succinates, benzoates and salts with amino acids such as glutamic acid. These salts may be prepared by methods known to those skilled in art. Also included are base addition salts such as sodium, potassium, calcium, zinc, magnesium, ammonium, piperidine, piperazine, organic amino, or a similar salt. When compounds of the present disclosure contain relatively basic functionalities, acid addition salts can be obtained by contacting the neutral form of such compounds with a sufficient amount of the desired acid, either neat or in a suitable inert solvent or by ion exchange. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present disclosure contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

In addition to salt forms, the present disclosure provides compounds, which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

The term “protecting group” refers to chemical moieties that block some or all reactive moieties of a compound and prevent such moieties from participating in chemical reactions until the protective group is removed, for example, those moieties listed and described in T. W. Greene, P. G. M. Wuts, Protective Groups in Organic Synthesis, 3rd ed. John Wiley & Sons (1999). It may be advantageous, where different protecting groups are employed, that each (different) protective group be removable by a different means. Protective groups that are cleaved under totally disparate reaction conditions allow differential removal of such protecting groups. For example, protective groups can be removed by acid, base, and hydrogenolysis. Groups such as trityl, dimethoxytrityl, acetal and tert-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, without limitation, methyl, ethyl, and acetyl in the presence of amines blocked with acid labile groups such as tert-butyl carbamate or with carbamates that are both acid and base stable but hydrolytically removable.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be blocked with oxidatively-removable protective groups such as 2,4-dimethoxybenzyl, while co-existing amino groups may be blocked with fluoride labile silyl carbamates.

Allyl blocking groups are useful in the presence of acid- and base-protecting groups since the former are stable and can be subsequently removed by metal or pi-acid catalysts. For example, an allyl-blocked carboxylic acid can be deprotected with a palladium(O)—catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

Typical blocking/protecting groups include, but are not limited to the following moieties:

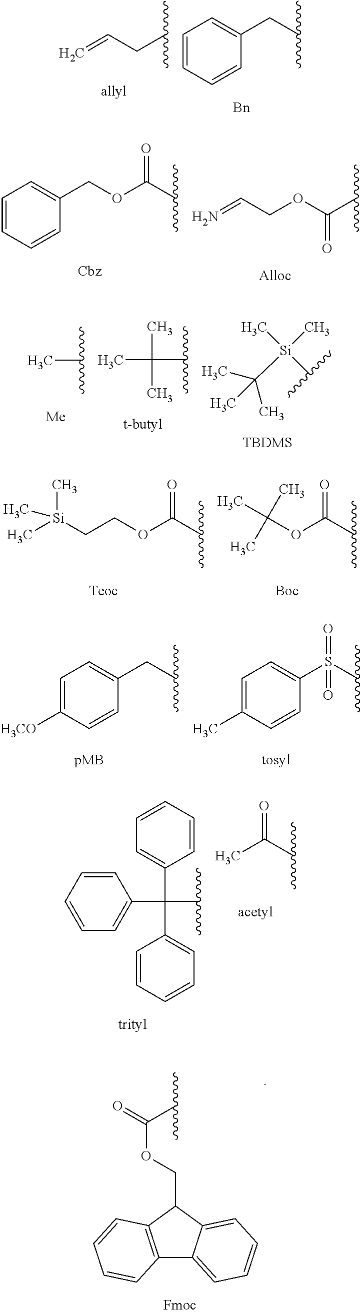

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of ordinary skill in the art depending on the desired properties sought to be obtained by the presently disclosed subject matter. For example, the term “about,” when referring to a value can be meant to encompass variations of, in some embodiments, ±100% in some embodiments±50%, in some embodiments±20%, in some embodiments±10%, in some embodiments±5%, in some embodiments±1%, in some embodiments±0.5%, and in some embodiments±0.10% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration and are not to be construed as limiting in any manner to make compounds of the disclosure by other methods.

### Synthesis of Representative Spirooxindole Acids of Formula (IA)

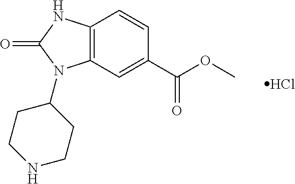 [SMILES: COC(=O)c1ccc2[nH]c(=O)n(C3CCNCC3)c2c1 ; Cl]

### Intermediate A: Methyl 2-oxospiro[indoline-3,4′-piperidine]-5-carboxylate hydrochloride

To a suspension of 1′-tert-butoxycarbonyl-2-oxospiro[indoline-3,4′-piperidine]-5-carboxylic acid (766 mg, 2.21 mmol) in methanol (10 mL) at 0° C. was added thionyl chloride (1.9 mL, 26 mmol). The resulting mixture was allowed to reach room temperature with stirring for 18 h. The solvent was removed in vacuo to give methyl 2-oxospiro[indoline-3,4′-piperidine]-5-carboxylate hydrochloride (600 mg, 2.02 mmol, 91.4% yield) as a yellow solid.

¹H NMR (400 MHz, DMSO-d₆) δ ppm 11.03 (s, 1H) 8.87 (br. s., 2H) 7.91 (dd, J 8.08, 1.77 Hz, 1H) 7.80 (d, J=1.52 Hz, 1H) 7.01 (d, J=8.34 Hz, 1H) 3.84 (s, 3H) 3.44-3.54 (m, 2H) 3.29 (d, J=13.14 Hz, 2H) 2.11-2.21 (m, 2H) 1.93 (d, J 14.40 Hz, 2H)

LCMS: [M+1]=261, rt=1.40 min.

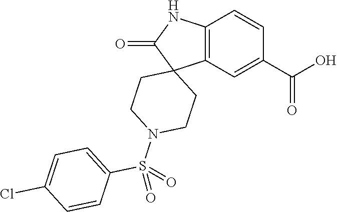 [SMILES: O=C(O)c1ccc2c(c1)C1(CCN(S(=O)(=O)c3ccc(Cl)cc3)CC1)C(=O)N2]

### Example 1: 1′-((4-chlorophenyl)sulfonyl)-2-oxospiro[indoline-3,4′-piperidine]-5-carboxylic acid

### Methyl 1′-(4-chlorophenyl)sulfonyl-2-oxospiro[indoline-3,4′-piperidine]-5-carboxylate

To a suspension of methyl 2-oxospiro[indoline-3,4′-piperidine]-5-carboxylate hydrochloride (50.0 mg, 0.169 mmol), 4-chlorobenzenesulfonyl chloride (39.1 mg, 0.185 mmol) in chloroform (5 mL) was added DIPEA (90.2 μL, 0.506 mmol). The resulting mixture was stirred at room temperature for 30 min. The solvent was removed with a stream of N₂ and the residue triturated with water/MeOH (3:1) for 18 h. The solid was collected by filtration, washed with water and dried to give methyl 1′-(4-chlorophenyl)sulfonyl-2-oxospiro[indoline-3,4′-piperidine]-5-carboxylate (49.5 mg, 0.114 mmol, 67.5% yield) as a white solid.

¹H NMR (400 MHz, DMSO-d₆) δ ppm 10.83 (s, 1H) 7.83-7.88 (m, 3H) 7.77-7.82 (m, 2H) 7.73 (s, 1H) 6.93 (d, J=8.34 Hz, 1H) 3.81 (s, 3H) 3.37-3.45 (m, 2H) 3.23 (t, J=9.35 Hz, 2H) 1.92-2.02 (m, 2H) 1.79 (d, J=14.15 Hz, 2H)

LCMS: [M+1]=435/437, rt=1.32 min (lipophilic method).

### 1′-((4-chlorophenyl)sulfonyl)-2-oxospiro[indoline-3,4′-piperidine]-5-carboxylic acid

A suspension of methyl 1′-(4-chlorophenyl)sulfonyl-2-oxospiro[indoline-3,4′-piperidine]-5-carboxylate (49.5 mg, 0.114 mmol) and lithium hydroxide monohydrate (4.8 mg, 0.11 mmol) in THF (4 mL) and water (0.4 mL) was stirred at room temperature for 21 h, then at 40° C. for 28 h, then at 3 days at room temperature. The solvent was removed under a stream of N₂, taken up in water, filtered through a syringe filter and acidified by addition of 6 N HCl. After stirring for 18 h, the solid was collected by filtration, washed with water and dried to give 1′-((4-chlorophenyl)sulfonyl)-2-oxospiro[indoline-3,4′-piperidine]-5-carboxylic acid (38.4 mg, 0.0912 mmol, 80.2% yield) as a beige solid.

¹H NMR (400 MHz, DMSO-d₆) δ ppm 12.70 (br. s., 1H) 10.77 (s, 1H) 7.71-7.87 (m, 6H) 6.90 (d, J=8.08 Hz, 1H) 3.44 (d, J=11.87 Hz, 2H) 3.12-3.25 (m, 2H) 1.97 (t, J=10.36 Hz, 2H) 1.79 (d, J=13.90 Hz, 2H)

LCMS: [M+1]=421/423, rt=2.20 min.

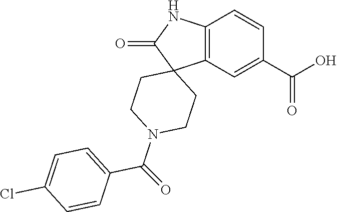 [SMILES: O=C(O)c1ccc2c(c1)C1(CCN(C(=O)c3ccc(Cl)cc3)CC1)C(=O)N2]

### Example 2: 1′-(4-chlorobenzoyl)-2-oxospiro[indoline-3,4′-piperidine]-5-carboxylic acid

### Methyl 1′-(4-chlorobenzoyl)-2-oxospiro[indoline-3,4′-piperidine]-5-carboxylate

To a suspension of methyl 2-oxospiro[indoline-3,4′-piperidine]-5-carboxylate hydrochloride (52 mg, 0.175 mmol), 4-chlorobenzoic acid (30.2 mg, 0.193 mmol) and HBTU (73.1 mg, 0.193 mmol) in chloroform (5 mL) was added DIPEA (93.8 μL, 0.526 mmol). the resulting mixture was stirred at room temperature for 2.5 h. The solvent was removed with a stream of N₂ and the residue triturated with water/MeOH (3:1) for 1 h. The solid was collected by filtration, washed with water and dried to give a solid which was purified by automated normal-phase chromatography (0-100% EtOAc/heptane, 4 g silica gel cartridge) to give methyl 1′-(4-chlorobenzoyl)-2-oxospiro[indoline-3,4′-piperidine]-5-carboxylate (52.4 mg, 0.131 mmol, 74.9% yield) as a white gummy solid.

¹H NMR (400 MHz, DMSO-d₆) δ ppm 10.86 (s, 1H) 8.07 (d, J=1.52 Hz, 1H) 7.88 (dd, J=8.21, 1.64 Hz, 1H) 7.56-7.60 (m, 2H) 7.51-7.55 (m, 2H) 6.97 (d, J=8.08 Hz, 1H) 4.20 (br. s., 1H) 3.83 (s, 3H) 3.76 (d, J=9.85 Hz, 2H) 3.50 (br. s., 1H) 1.94-2.04 (m, 2H) 1.80 (br. s., 1H) 1.66 (br. s., 1H)

LCMS: [M+1]=399/401, rt=2.34 min.

### 1′-(4-chlorobenzoyl)-2-oxospiro[indoline-3,4′-piperidine]-5-carboxylic acid

A suspension of methyl 1′-(4-chlorobenzoyl)-2-oxospiro[indoline-3,4′-piperidine]-5-carboxylate (52.4 mg, 0.131 mmol) and lithium hydroxide monohydrate (5.5 mg, 0.131 mmol) in THF (4 mL) and water (0.4 mL) was stirred at room temperature for 21 h, then at 40° C. for 21 h. The solvent was removed under a stream of N₂ to give a residue which was taken up in water then filtered through a syringe filter. The solution was acidified by addition of 6 N HCl and stirred for 1 h. The solid was collected by filtration, washed with water and air-dried to give a residue which was purified by automated normal-phase chromatography (0-20% MeOH/DCM, 4 g silica gel cartridge) to give 1′-(4-chlorobenzoyl)-2-oxospiro[indoline-3,4′-piperidine]-5-carboxylic acid (4.7 mg, 0.012 mmol, 9.3% yield) as a white solid.

¹H NMR (400 MHz, DMSO-d₆) δ ppm 12.72 (br. s., 1H) 10.82 (s, 1H) 8.05 (s, 1H) 7.86 (d, J=8.08 Hz, 1H) 7.55-7.59 (m, 2H) 7.51-7.55 (m, 2H) 6.94 (d, J=8.08 Hz, 1H) 4.17 (br. s., 1H) 3.78 (t, J=10.61 Hz, 2H) 3.50 (br. s., 1H) 1.90-2.01 (m, 2H) 1.80 (br. s., 1H) 1.67 (br. s., 1H)

LCMS: [M+1]=385/387, rt=2.04 min.

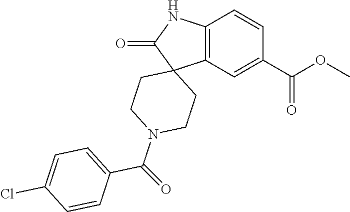 [SMILES: COC(=O)c1ccc2c(c1)C1(CCN(C(=O)c3ccc(Cl)cc3)CC1)C(=O)N2]

### Example 3: Methyl 1′-(4-chlorobenzoyl)-2-oxospiro[indoline-3,4′-piperidine]-5-carboxylate

To a suspension of methyl 2-oxospiro[indoline-3,4′-piperidine]-5-carboxylate hydrochloride (51.8 mg, 0.175 mmol), 4-chlorobenzoic acid (30.1 mg, 0.192 mmol) and HBTU (72.8 mg, 0.192 mmol) in chloroform (5 mL) was added DIPEA (93.5 μL, 0.524 mmol). The resulting mixture was stirred at room temperature for 17 h. The solvent was removed with a stream of N₂ and the residue triturated with water/MeOH (3:1) for 18 h. The solid was collected by filtration, washed with water and dried to give methyl 1′-(4-chlorobenzoyl)-2-oxospiro[indoline-3,4′-piperidine]-5-carboxylate (63.0 mg, 0.158 mmol, 90.5% yield) as a white solid.

¹H NMR (400 MHz, DMSO-d₆) δ ppm 10.86 (s, 1H) 8.07 (s, 1H) 7.88 (d, J=8.34 Hz, 1H) 7.56-7.59 (m, 2H) 7.51-7.55 (m, 2H) 6.97 (d, J=8.08 Hz, 1H) 4.22 (br. s., 1H) 3.83 (s, 3H) 3.77 (br. s., 2H) 3.50 (br. s., 1H) 1.94-2.04 (m, 2H) 1.77 (br. s., 1H) 1.67 (br. s., 1H)

LCMS: [M+1]=399/401, rt=2.35 min.

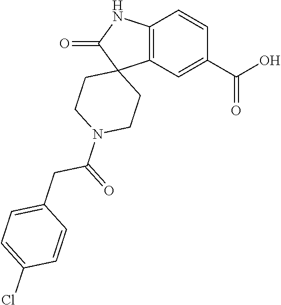 [SMILES: O=C(O)c1ccc2c(c1)C1(CCN(C(=O)Cc3ccc(Cl)cc3)CC1)C(=O)N2]

### Example 4: 1′-(2-(4-chlorophenyl)acetyl)-2-oxospiro[indoline-3,4′-piperidine]-5-carboxylic acid

### Methyl 1′-(2-(4-chlorophenyl)acetyl)-2-oxospiro[indoline-3,4′-piperidine]-5-carboxylate

To a suspension of methyl 2-oxospiro[indoline-3,4′-piperidine]-5-carboxylate hydrochloride (55.0 mg, 0.185 mmol), 4-chlorophenylacetic acid (34.8 mg, 0.204 mmol) and HBTU (77.3 mg, 0.204 mmol) in chloroform (5 mL) was added DIPEA (99.2 μL, 0.556 mmol). The resulting mixture was stirred at room temperature for 20 h. The solvent was removed with a stream of N₂ and the residue triturated with water/MeOH (3:1) for 18 h. The solid was collected by filtration, washed with water and dried to give methyl 1′-[2-(4-chlorophenyl)acetyl]-2-oxospiro[indoline-3,4′-piperidine]-5-carboxylate (49.5 mg, 0.120 mmol, 64.7% yield) as a white solid.

¹H NMR (400 MHz, DMSO-d₆) δ ppm 10.86 (s, 1H) 7.85-7.90 (m, 2H) 7.37-7.43 (m, 2H) 7.29-7.34 (m, 2H) 6.96 (d, J=8.59 Hz, 1H) 3.98-4.07 (m, 1H) 3.75-3.89 (m, 7H) 3.57-3.67 (m, 1H) 1.64-1.80 (m, 4H)

LCMS: [M+1]=413/415, rt=1.19 min (lipophilic method).

### 1′-(2-(4-chlorophenyl)acetyl)-2-oxospiro[indoline-3,4′-piperidine]-5-carboxylic acid

A suspension of methyl 1′-[2-(4-chlorophenyl)acetyl]-2-oxo-spiro[indoline-3,4′-piperidine]-5-carboxylate (49.5 mg, 0.120 mmol) and lithium hydroxide monohydrate (5.0 mg, 0.120 mmol) in THF (4 mL) and water (0.4 mL) was stirred at room temperature for 6 days. The solvent was removed with a stream of N₂. The residue was taken up in water, filtered through a syringe filter, acidified by addition of 6 N HCl and stirred for 3 h. The solid was collected by filtration, washed with water and dried to give a yellow solid which was purified by automated normal-phase chromatography (0-15% MeOH/DCM, 4 g silica gel cartridge) to give 1′-[2-(4-chlorophenyl)acetyl]-2-oxospiro[indoline-3,4′-piperidine]-5-carboxylic acid (15.0 mg, 0.0376 mmol, 31.4% yield) as a colorless film.

¹H NMR (400 MHz, DMSO-d₆) δ ppm 12.73 (br. s., 1H) 10.82 (s, 1H) 7.83-7.90 (m, 2H) 7.37-7.42 (m, 2H) 7.28-7.33 (m, 2H) 6.94 (d, J=7.83 Hz, 1H) 3.91-4.04 (m, 1H) 3.73-3.89 (m, 4H) 3.60-3.71 (m, 1H) 1.63-1.83 (m, 4H)

LCMS: [M+1]=399/401, rt=2.05 min.

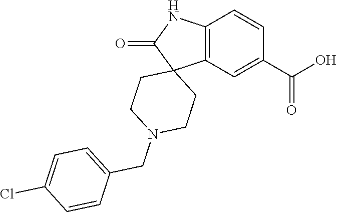 [SMILES: O=C(O)c1ccc2c(c1)C1(CCN(Cc3ccc(Cl)cc3)CC1)C(=O)N2]

### Example 5: 1′-(4-chlorobenzyl)-2-oxospiro[indoline-3,4′-piperidine]-5-carboxylic acid

### Methyl 1′-(4-chlorobenzyl)-2-oxospiro[indoline-3,4′-piperidine]-5-carboxylate

A suspension of methyl 2-oxospiro[indoline-3,4′-piperidine]-5-carboxylate hydrochloride (56.5 mg, 0.190 mmol), 4-chlorobenzaldehyde (29.4 mg, 0.209 mmol) and sodium triacetoxyborohydride (121 mg, 0.571 mmol) in chloroform (5 mL) was stirred at room temperature for 26 h. The solvent was removed with a stream of N₂ and the residue was partitioned between water and EtOAc. The solvent was removed from the organic layer with a stream of N₂ to give a residue which was purified by automated normal-phase chromatography (0-20% MeOH/DCM, 4 g silica gel cartridge) to give methyl 1′-[(4-chlorophenyl)methyl]-2-oxo-spiro[indoline-3,4′-piperidine]-5-carboxylate acetate (72.0 mg, 0.162 mmol, 85.0% yield) as a colorless gum.

¹H NMR (400 MHz, DMSO-d₆) δ ppm 12.02 (br. s., 1H) 10.82 (s, 1H) 7.96 (s, 1H) 7.87 (d, J=8.08 Hz, 1H) 7.42 (s, 4H) 6.96 (d, J=8.08 Hz, 1H) 3.83 (s, 3H) 3.63 (s, 2H) 2.83 (br. s., 2H) 2.56 (br. s., 2H) 1.92 (s, 3H) 1.81 (br. s., 2H) 1.71 (br. s., 2H)

LCMS: [M+1]=385/387, rt=2.01 min.

### 1′-(4-chlorobenzyl)-2-oxospiro[indoline-3,4′-piperidine]-5-carboxylic acid

To a solution of methyl 1′-[(4-chlorophenyl)methyl]-2-oxospiro[indoline-3,4′-piperidine]-5-carboxylate acetate (72.0 mg, 0.162 mmol) in methanol (5 mL) was added 1 N NaOH (485 μL, 0.485 mmol). The resulting mixture was stirred at room temperature for 18 h, then at 50° C. for 31 h. After cooling, 6 N HCl (27 μL, 1 eq.) was added and the solvent removed with a stream of N₂ to give a residue which was purified by automated normal-phase chromatography (0-40% MeOH/DCM, 4 g silica gel cartridge) to give 1′-[(4-chlorophenyl)methyl]-2-oxospiro[indoline-3,4′-piperidine]-5-carboxylic acid (29.1 mg, 0.0785 mmol, 48.5% yield) as a white solid.

¹H NMR (400 MHz, DMSO-d₆) δ ppm 10.79 (s, 1H) 7.98 (s, 1H) 7.86 (dd, J=8.21, 1.64 Hz, 1H) 7.37-7.46 (m, 4H) 6.94 (d, J=8.08 Hz, 1H) 3.63 (br. s., 2H) 2.84 (br. s., 2H) 2.56 (br. s., 2H) 1.78-1.88 (m, 2H) 1.71 (br. s., 2H)

LCMS: [M+1]=371/373, rt=1.99 min.

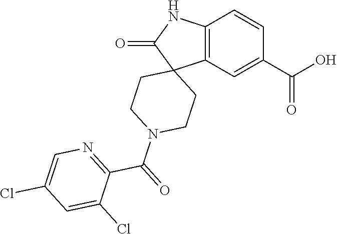 [SMILES: O=C(O)c1ccc2c(c1)C1(CCN(C(=O)c3ncc(Cl)cc3Cl)CC1)C(=O)N2]

### Example 6: 1′-(3,5-dichloropicolinoyl)-2-oxospiro[indoline-3,4′-piperidine]-5-carboxylic acid

### Methyl 1′-(3,5-dichloropicolinoyl)-2-oxospiro[indoline-3,4′-piperidine]-5-carboxylate

To a suspension of methyl 2-oxospiro[indoline-3,4′-piperidine]-5-carboxylate hydrochloride (40.0 mg, 0.135 mmol), 3,5-dichloropyridine-2-carboxylic acid (28.5 mg, 0.148 mmol) and HBTU (56.2 mg, 0.148 mmol) in chloroform (5 mL) was added DIPEA (72.2 μL, 0.404 mmol). The resulting mixture was stirred at room temperature for 21 h. The solvent was removed with a stream of N₂ and the residue triturated with water/MeOH (5:1) for 18 h. The solid was collected by filtration, washed with water and dried to give methyl 1′-(3,5-dichloropicolinoyl)-2-oxospiro[indoline-3,4′-piperidine]-5-carboxylate (29.2 mg, 0.0672 mmol, 49.9% yield) as a white solid.

¹H NMR (400 MHz, DMSO-d₆) δ ppm 10.93 (s, 1H) 8.71 (d, J=2.02 Hz, 1H) 8.44 (d, J=2.02 Hz, 1H) 7.87-7.93 (m, 2H) 6.99 (d, J=8.08 Hz, 1H) 4.10-4.22 (m, 1H) 3.79-3.92 (m, 4H) 3.63-3.74 (m, 1H) 3.23-3.32 (m, 1H) 1.69-1.96 (m, 4H)

LCMS: [M+1]=434/436, rt=2.22 min.

### 1′-(3,5-dichloropicolinoyl)-2-oxospiro[indoline-3,4′-piperidine]-5-carboxylic acid

To a solution of methyl 1′-(3,5-dichloropyridine-2-carbonyl)-2-oxo-spiro[indoline-3,4′-piperidine]-5-carboxylate (29.2 mg, 0.0672 mmol) in methanol (5 mL) was added 1 N NaOH (135 μL, 0.135 mmol). The resulting mixture was stirred at room temperature for 22 h, then at 50° C. for 48 h. The solvent was removed with a stream of N₂ to give a residue which was purified by automated normal-phase chromatography (0-20% MeOH/DCM, 4 g silica gel cartridge) to give 1′-(3,5-dichloropicolinoyl)-2-oxospiro[indoline-3,4′-piperidine]-5-carboxylic acid (18.0 mg, 0.0428 mmol, 63.7% yield) as a white solid.

¹H NMR (400 MHz, DMSO-d₆) δ ppm 12.76 (br. s., 1H) 10.89 (s, 1H) 8.71 (d, J=2.02 Hz, 1H) 8.44 (d, J=2.02 Hz, 1H) 7.84-7.90 (m, 2H) 6.96 (d, J=8.34 Hz, 1H) 4.09-4.20 (m, 1H) 3.84-3.93 (m, 1H) 3.63-3.73 (m, 1H) 3.28 (d, J=13.89 Hz, 1H) 1.86 (d, J=4.80 Hz, 2H) 1.71-1.80 (m, 2H)

LCMS: [M+1]=420/422/424, rt=1.90 min.

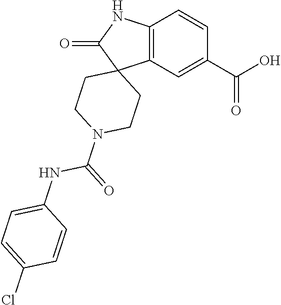 [SMILES: O=C(O)c1ccc2c(c1)C1(CCN(C(=O)Nc3ccc(Cl)cc3)CC1)C(=O)N2]

### Example 7: 1′-((4-chlorophenyl)carbamoyl)-2-oxospiro[indoline-3,4′-piperidine]-5-carboxylic acid

### Methyl 1′-((4-chlorophenyl)carbamoyl)-2-oxospiro[indoline-3,4′-piperidine]-5-carboxylate

To a suspension of methyl 2-oxospiro[indoline-3,4′-piperidine]-5-carboxylate hydrochloride (38.0 mg, 0.128 mmol) and 4-chlorophenyl isocyanate (20.6 mg, 0.135 mmol) was added DIPEA (45.7 μL, 0.256 mmol). The resulting mixture was stirred at room temperature for 3 h. The solvent was removed with a stream of N₂ and the residue was triturated with water/MeOH (4:1) for 18 h. The solid was collected by filtration and dried to give methyl 1′-((4-chlorophenyl)carbamoyl)-2-oxospiro[indoline-3,4′-piperidine]-5-carboxylate (48.0 mg, 0.116 mmol, 90.6% yield) as a white solid, and was used without further purification.

¹H NMR (400 MHz, DMSO-d₆) δ ppm 10.89 (s, 1H) 8.73 (s, 1H) 7.96 (d, J=1.52 Hz, 1H) 7.89 (dd, J=8.08, 1.52 Hz, 1H) 7.53-7.57 (m, 2H) 7.28-7.33 (m, 2H) 6.97-7.01 (m, 1H) 3.77-3.85 (m, 7H) 1.72-1.88 (m, 4H)

LCMS: [M+1]=414/416, rt=2.35 min.

### 1′-((4-chlorophenyl)carbamoyl)-2-oxospiro[indoline-3,4′-piperidine]-5-carboxylic acid

A mixture of methyl 1′-[(4-chlorophenyl)carbamoyl]-2-oxospiro[indoline-3,4′-piperidine]-5-carboxylate (48.0 mg, 0.116 mmol) and 1 N NaOH (232 μL, 0.232 mmol) was stirred at 50° C. for 75 h. The contents were acidified by addition of 6 N HCl, then the solvent was removed with a stream of N₂. The residue which was purified by automated normal-phase chromatography (0-100% MeOH/DCM, 4 g silica gel cartridge) to give 1′-((4-chlorophenyl)carbamoyl)-2-oxospiro[indoline-3,4′-piperidine]-5-carboxylic acid (5.5 mg, 0.014 mmol, 12% yield) as a white solid.

¹H NMR (400 MHz, DMSO-d₆) δ ppm 12.73 (br. s., 1H) 10.85 (s, 1H) 8.74 (s, 1H) 7.93 (d, J=1.52 Hz, 1H) 7.87 (dd, J=8.08, 1.52 Hz, 1H) 7.52-7.57 (m, 2H) 7.27-7.32 (m, 2H) 6.96 (d, J=8.08 Hz, 1H) 3.81 (t, J=5.68 Hz, 4H) 1.71-1.86 (m, 4H)

LCMS: [M+1]=400/402, rt=2.03 min.

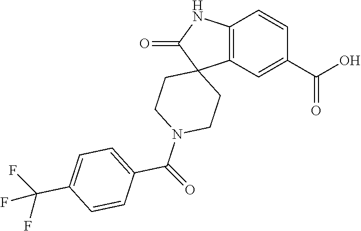 [SMILES: O=C(O)c1ccc2c(c1)C1(CCN(C(=O)c3ccc(C(F)(F)F)cc3)CC1)C(=O)N2]

### Example 8: 2-oxo-1′-(4-(trifluoromethyl)benzoyl)spiro[indoline-3,4′-piperidine]-5-carboxylic acid

### Methyl 2-oxo-1′-(4-(trifluoromethyl)benzoyl)spiro[indoline-3,4′-piperidine]-5-carboxylate

To a suspension of methyl 2-oxospiro[indoline-3,4′-piperidine]-5-carboxylate hydrochloride (57.0 mg, 0.192 mmol), 4-(trifluoromethyl)benzoic acid (62.7 mg, 0.330 mmol) and HBTU (80.1 mg, 0.211 mmol) in Chloroform (5 mL) was added DIPEA (103 μL, 0.576 mmol). The resulting mixture was stirred at room temperature for 2 h. The solvent was removed with a stream of N₂ and the residue triturated with water/MeOH (9:1) for 18 h. The solid was collected by filtration, washed with water and dried to give methyl 2-oxo-1′-(4-(trifluoromethyl)benzoyl)spiro[indoline-3,4′-piperidine]-5-carboxylate (48.2 mg, 0.112 mmol, 58.0% yield) as a white solid.

¹H NMR (400 MHz, DMSO-d₆) δ ppm 10.88 (s, 1H) 8.09 (s, 1H) 7.88 (dd, J=8.21, 1.64 Hz, 1H) 7.83-7.86 (m, 2H) 7.75-7.79 (m, 2H) 6.97 (d, J=8.08 Hz, 1H) 4.27 (d, J=12.63 Hz, 1H) 3.83 (s, 3H) 3.76 (d, J=11.37 Hz, 2H) 3.43 (d, J 12.13 Hz, 1H) 2.02 (d, J=11.12 Hz, 2H) 1.80 (d, J=14.15 Hz, 1H) 1.64 (d, J 13.39 Hz, 1H)

¹⁹F NMR (376 MHz, DMSO-d₆) δ ppm −161.04 (s, 3F)

LCMS: [M+1]=433, rt=2.44 min.

### 2-oxo-1′-(4-(trifluoromethyl)benzoyl)spiro[indoline-3,4′-piperidine]-5-carboxylic acid

To a solution of methyl 2-oxo-1′-(4-(trifluoromethyl)benzoyl)spiro[indoline-3,4′-piperidine]-5-carboxylate (48.2 mg, 0.112 mmol) in methanol (5 mL) was added 1 N NaOH (223 μL, 0.223 mmol). The resulting mixture was stirred at room temperature for 16 h, then at 50° C. for 79 h. The solvent was removed under a stream of N₂, and the residue taken up in water and filtered through a syringe filter. The solution was acidified by addition of 6 N HCl and stirred for 18 h. The solid was collected by filtration, washed with water and dried under vacuum to give 2-oxo-1′-(4-(trifluoromethyl)benzoyl)spiro[indoline-3,4′-piperidine]-5-carboxylic acid (32.4 mg, 0.0774 mmol, 69.5% yield) as an off-white solid.

¹H NMR (400 MHz, DMSO-d₆) δ ppm 12.72 (br. s., 1H) 10.83 (s, 1H) 8.06 (s, 1H) 7.73-7.90 (m, 5H) 6.94 (d, J=8.08 Hz, 1H) 4.22 (d, J=11.62 Hz, 1H) 3.77 (t, J=12.00 Hz, 2H) 3.43 (d, J=13.14 Hz, 1H) 1.98 (d, J=10.86 Hz, 2H) 1.80 (d, J 13.39 Hz, 1H) 1.65 (d, J=14.15 Hz, 1H)

¹⁹F NMR (376 MHz, DMSO-d₆) δ ppm −161.05 (s, 3F)

LCMS: [M+1]=419, rt=2.14 min.

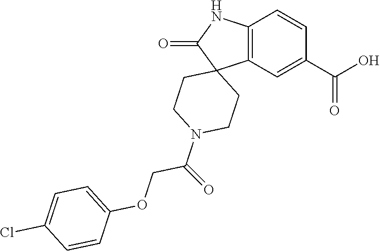 [SMILES: O=C(O)c1ccc2c(c1)C1(CCN(C(=O)COc3ccc(Cl)cc3)CC1)C(=O)N2]

### Example 9: 1′-(2-(4-chlorophenoxy)acetyl)-2-oxospiro[indoline-3,4′-piperidine]-5-carboxylic acid

### Methyl 1′-(2-(4-chlorophenoxy)acetyl)-2-oxospiro[indoline-3,4′-piperidine]-5-carboxylate

To a suspension of methyl 2-oxospiro[indoline-3,4′-piperidine]-5-carboxylate hydrochloride (42.0 mg, 0.142 mmol), (4-chlorophenoxy)acetic acid (29.1 mg, 0.156 mmol) and HBTU (59.0 mg, 0.156 mmol) in chloroform (5 mL) was added DIPEA (75.8 μL, 0.425 mmol). The resulting mixture was stirred at room temperature for 2 h. The solvent was removed with a stream of N₂ and the residue triturated with water/MeOH (7:1) for 4 h. The solid was collected by filtration, washed with water and dried to give methyl 1′-[2-(4-chlorophenoxy)acetyl]-2-oxospiro[indoline-3,4′-piperidine]-5-carboxylate (41.2 mg, 0.0961 mmol, 67.9% yield) as a white solid.

¹H NMR (400 MHz, DMSO-d₆) δ ppm 10.88 (s, 1H) 7.93 (d, J=1.52 Hz, 1H) 7.88 (dd, J=8.08, 1.77 Hz, 1H) 7.32-7.37 (m, 2H) 6.99-7.02 (m, 2H) 6.97 (d, J 8.08 Hz, 1H) 4.88-5.00 (m, 2H) 4.02 (d, J=12.63 Hz, 1H) 3.78-3.88 (m, 4H) 3.68-3.76 (m, 1H) 3.57-3.67 (m, 1H) 2.02 (t, J=9.85 Hz, 1H) 1.67-1.83 (m, 3H)

LCMS: [M+1]=429/431, rt=2.40 min.

### 1′-(2-(4-chlorophenoxy)acetyl)-2-oxospiro[indoline-3,4′-piperidine]-5-carboxylic acid

A suspension of methyl 1′-[2-(4-chlorophenoxy)acetyl]-2-oxo-spiro[indoline-3,4′-piperidine]-5-carboxylate (41.2 mg, 0.961 mmol) in methanol (5 mL) was treated with 1 N NaOH (192 μL, 0.192 mmol) and stirred at 50° C. for 102 h. The solvent was removed under a stream of N₂, and the residue taken up in water and filtered through a syringe filter. The solution was acidified by addition of 6 N HCl and stirred for 18 h. The solid was collected by filtration, washed with water and dried under vacuum to give 1′-(2-(4-chlorophenoxy)acetyl)-2-oxospiro[indoline-3,4′-piperidine]-5-carboxylic acid (16.4 mg, 0.0395 mmol, 41.2% yield) as an off-white solid.

¹H NMR (400 MHz, DMSO-d₆) δ ppm 12.69 (br. s., 1H) 10.82 (br. s., 1H) 7.92 (br. s., 1H) 7.86 (d, J=7.23 Hz, 1H) 7.34 (d, J=8.49 Hz, 2H) 7.00 (d, J=8.65 Hz, 2H) 6.95 (d, J=7.70 Hz, 1H) 4.88-5.00 (m, 2H) 3.97 (d, J=12.42 Hz, 1H) 3.84 (br. s., 1H) 3.62-3.76 (m, 2H) 1.99 (br. s., 1H) 1.68-1.82 (m, 3H)

LCMS: [M+1]=415/417, rt=2.10 min.

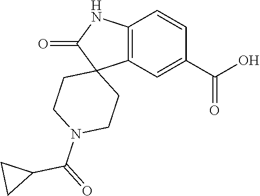 [SMILES: O=C(O)c1ccc2c(c1)C1(CCN(C(=O)C3CC3)CC1)C(=O)N2]

### Example 10: 1′-(cyclopropanecarbonyl)-2-oxospiro[indoline-3,4′-piperidine]-5-carboxylic acid

### Methyl 1′-(cyclopropanecarbonyl)-2-oxospiro[indoline-3,4′-piperidine]-5-carboxylate

To a suspension of methyl 2-oxospiro[indoline-3,4′-piperidine]-5-carboxylate hydrochloride (45.4 mg, 0.153 mmol) in chloroform (5 mL) was added cyclopropanecarbonyl chloride (13.9 μL, 0.153 mmol) followed by DIPEA (81.9 μL, 0.459 mmol). The resulting mixture was stirred at room temperature for 90 min. The solvent was removed with a stream of N₂ and the residue triturated with water/MeOH (9:1) for 18 h. The solid was collected by filtration, washed with water and dried to give methyl 1′-(cyclopropanecarbonyl)-2-oxospiro[indoline-3,4′-piperidine]-5-carboxylate (39.2 mg, 0.119 mmol, 78.0% yield) as a white solid.

¹H NMR (400 MHz, DMSO-d₆) δ ppm 10.89 (s, 1H) 7.96 (d, J=1.52 Hz, 1H) 7.89 (dd, J=8.21, 1.64 Hz, 1H) 6.98 (d, J=8.34 Hz, 1H) 3.93-4.05 (m, 3H) 3.82 (s, 3H) 3.67 (br. s., 1H) 2.00-2.08 (m, 1H) 1.88 (br. s., 1H) 1.64-1.83 (m, 3H) 0.83 (br. s., 1H) 0.70-0.80 (m, 3H)

LCMS: [M+1]=329, rt=1.92 min.

### 1′-(cyclopropanecarbonyl)-2-oxospiro[indoline-3,4′-piperidine]-5-carboxylic acid

A suspension of methyl 1′-(cyclopropanecarbonyl)-2-oxospiro[indoline-3,4′-piperidine]-5-carboxylate (39.2 mg, 0.119 mmol) in methanol (5 mL) was treated with 1 N NaOH (239 μL, 0.239 mmol) and stirred at 50° C. for 60 h. The solvent was removed under a stream of N₂, and the residue taken up in water and filtered through a syringe filter. The solution was acidified by addition of 6 N HCl and stirred for 18 h. The solid was collected by filtration, washed with water and dried under vacuum to give 1′-(cyclopropanecarbonyl)-2-oxospiro[indoline-3,4′-piperidine]-5-carboxylic acid (28.3 mg, 0.0900 mmol, 75.4% yield) as a white solid.

¹H NMR (400 MHz, DMSO-d₆) δ ppm 12.68 (br. s., 1H) 10.82 (br. s., 1H) 7.93 (br. s., 1H) 7.87 (d, J=7.70 Hz, 1H) 6.96 (d, J=7.86 Hz, 1H) 3.89-4.07 (m, 3H) 3.71 (br. s., 1H) 2.03 (br. s., 1H) 1.66-1.92 (m, 4H) 0.70-0.86 (m, 4H)

LCMS: [M+1]=315, rt=1.59 min.

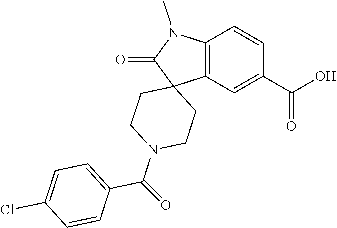 [SMILES: CN1C(=O)C2(CCN(C(=O)c3ccc(Cl)cc3)CC2)c2cc(C(=O)O)ccc21]

### Example 11: 1′-(4-chlorobenzoyl)-1-methyl-2-oxospiro[indoline-3,4′-piperidine]-5-carboxylic acid

A suspension of sodium hydride (6.0 mg, 0.15 mmol) in THF (2 mL) was stirred at room temperature for 10 min, then a solution of methyl 1′-(4-chlorobenzoyl)-2-oxospiro[indoline-3,4′-piperidine]-5-carboxylate (50.0 mg, 0.125 mmol) and iodomethane (9.4 μL, 0.15 mmol) in THF (2 mL) was added portionwise over 5 min at room temperature and the resulting mixture stirred for 18 h. Added sodium hydride (6.0 mg, 0.15 mmol) and stirred an additional 24 h. The solvent was removed under a stream of N₂, and the residue taken up in water and filtered through a syringe filter. The solution was acidified by addition of 6 N HCl and stirred for 2 h. The solid was collected by filtration, washed with water and dried under vacuum to give 1′-(4-chlorobenzoyl)-1-methyl-2-oxo-spiro[indoline-3,4′-piperidine]-5-carboxylic acid (44.3 mg, 0.111 mmol, 88.6% yield) as a yellow solid.

¹H NMR (400 MHz, DMSO-d₆) δ ppm 12.76 (br. s., 1H) 8.09 (br. s., 1H) 7.95 (d, J 7.23 Hz, 1H) 7.58 (d, J=8.02 Hz, 2H) 7.50-7.55 (m, 2H) 7.14 (d, J=8.17 Hz, 1H) 4.22 (br. s., 1H) 3.77 (br. s., 2H) 3.51 (br. s., 1H) 3.18 (s, 3H) 1.95-2.05 (m, 2H) 1.77 (br. s., 1H) 1.65 (br. s., 1H)

LCMS: [M+1]=413, rt=2.22 min.

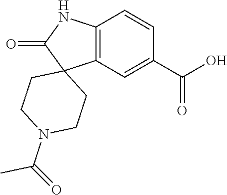 [SMILES: CC(=O)N1CCC2(CC1)C(=O)Nc1ccc(C(=O)O)cc12]

### Example 12: 1′-acetyl-2-oxospiro[indoline-3,4′-piperidine]-5-carboxylic acid

### Methyl 1′-acetyl-2-oxospiro[indoline-3,4′-piperidine]-5-carboxylate

To a suspension of methyl 2-oxospiro[indoline-3,4′-piperidine]-5-carboxylate hydrochloride (40.4 mg, 0.136 mmol) in chloroform (5 mL) was added acetyl chloride (9.7 μL, 0.136 mmol) followed by DIPEA (72.9 μL, 0.408 mmol). The resulting mixture was stirred at room temperature for 90 min. The solvent was removed with a stream of N₂ and the residue triturated with water/MeOH (7:1) for 18 h. The solid was collected by filtration, washed with water and dried to give methyl 1′-acetyl-2-oxospiro[indoline-3,4′-piperidine]-5-carboxylate (20.0 mg, 0.0662 mmol, 48.6% yield) as a beige solid.

¹H NMR (400 MHz, DMSO-d₆) δ ppm 10.87 (s, 1H) 7.95 (d, J=1.52 Hz, 1H) 7.88 (dd, J=8.21, 1.64 Hz, 1H) 6.97 (d, J=8.08 Hz, 1H) 3.99 (d, J=12.63 Hz, 1H) 3.82 (s, 4H) 3.68 (d, J=13.90 Hz, 1H) 3.54-3.63 (m, 1H) 2.08 (s, 3H) 1.89-1.98 (m, 1H) 1.62-1.79 (m, 3H)

LCMS: [M+1]=303, rt=1.72 min.

### 1′-acetyl-2-oxospiro[indoline-3,4′-piperidine]-5-carboxylic acid

A suspension of methyl 1′-acetyl-2-oxospiro[indoline-3,4′-piperidine]-5-carboxylate (20.0 mg, 0.0662 mmol) in methanol (5 mL) was treated with 1 N NaOH (132 μL, 0.132 mmol) and stirred at 50° C. for 123 h. The solvent was removed under a stream of N₂, and the residue taken up in water and filtered through a syringe filter. The solution was acidified by addition of 6 N HCl and stirred for 3 h. The solid was collected by filtration, washed with water and dried under vacuum to give 1′-acetyl-2-oxospiro[indoline-3,4′-piperidine]-5-carboxylic acid (10.1 mg, 0.0350 mmol, 52.9% yield) as an off-white solid.

¹H NMR (400 MHz, DMSO-d₆) δ ppm 12.67 (br. s., 1H) 10.80 (br. s., 1H) 7.92 (br. s., 1H) 7.86 (d, J=7.86 Hz, 1H) 6.95 (d, J=7.86 Hz, 1H) 3.95 (br. d, J=12.40 Hz, 1H) 3.80-3.88 (m, 1H) 3.60-3.71 (m, 2H) 2.08 (s, 3H) 1.87-1.95 (m, 1H) 1.64-1.79 (m, 3H)

LCMS: [M+1]=289, rt=1.39 min.

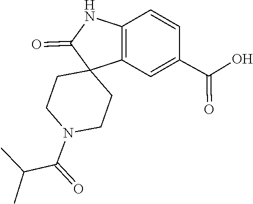 [SMILES: CC(C)C(=O)N1CCC2(CC1)C(=O)Nc1ccc(C(=O)O)cc12]

### Example 13: 1′-isobutyryl-2-oxospiro[indoline-3,4′-piperidine]-5-carboxylic acid

### Methyl 1′-isobutyryl-2-oxospiro[indoline-3,4′-piperidine]-5-carboxylic acid

To a suspension of methyl 2-oxospiro[indoline-3,4′-piperidine]-5-carboxylate hydrochloride (42.1 mg, 0.142 mmol) in chloroform (5 mL) was added isobutyryl chloride (14.9 μL, 0.142 mmol) followed by DIPEA (76.0 μL, 0.426 mmol). The resulting mixture was stirred at room temperature for 90 min. The solvent was removed with a stream of N₂ and the residue triturated with water/MeOH (9:1) for 42 h. The solid was collected by filtration, washed with water and dried to give methyl 1′-(2-methylpropanoyl)-2-oxospiro[indoline-3,4′-piperidine]-5-carboxylate (20.0 mg, 0.0605 mmol, 42.7% yield) as a white solid.

¹H NMR (400 MHz, DMSO-d₆) δ ppm 10.88 (s, 1H) 7.95 (d, J=1.52 Hz, 1H) 7.89 (dd, J=8.08, 1.77 Hz, 1H) 6.98 (d, J=8.34 Hz, 1H) 3.98 (d, J=13.64 Hz, 1H) 3.86 (d, J=9.85 Hz, 1H) 3.82 (s, 3H) 3.79 (br. s., 1H) 3.65 (t, J=8.72 Hz, 1H) 2.93 (dt, J=13.58, 6.73 Hz, 1H) 1.82-1.92 (m, 1H) 1.65-1.80 (m, 3H) 1.05 (dd, J=15.28, 6.69 Hz, 6H)

LCMS: [M+1]=331, rt=1.99 min.

### 1′-isobutyryl-2-oxospiro[indoline-3,4′-piperidine]-5-carboxylic acid

A suspension of methyl 1′-(2-methylpropanoyl)-2-oxospiro[indoline-3,4′-piperidine]-5-carboxylate (20.0 mg, 0.0605 mmol) in methanol (5 mL) was treated with 1 N NaOH (242 μL, 0.242 mmol) and stirred at 50° C. for 96 h. The solvent was removed under a stream of N₂, and the residue taken up in water and filtered through a syringe filter. The solution was acidified by addition of 6 N HCl and stirred for 3 h. The solid was collected by filtration, washed with water and dried under vacuum to give 1′-(2-methylpropanoyl)-2-oxospiro[indoline-3,4′-piperidine]-5-carboxylic acid (14.5 mg, 0.0458 mmol, 75.7% yield) as a white solid.

¹H NMR (400 MHz, DMSO-d₆) δ ppm 12.68 (br. s., 1H) 10.82 (br. s., 1H) 7.92 (br. s., 1H) 7.86 (d, J=7.70 Hz, 1H) 6.96 (d, J=7.39 Hz, 1H) 3.85-3.98 (m, 2H) 3.65-3.81 (m, 2H) 2.89-2.98 (m, 1H) 1.67-1.89 (m, 4H) 1.06 (br. d, J=10.80 Hz, 6H)

LCMS: [M+1]=317, rt=1.68 min.

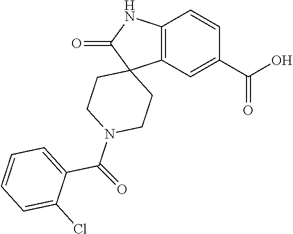 [SMILES: O=C(O)c1ccc2c(c1)C1(CCN(C(=O)c3ccccc3Cl)CC1)C(=O)N2]

### Example 14: 1′-(2-chlorobenzoyl)-2-oxospiro[indoline-3,4′-piperidine]-5-carboxylic acid

### Methyl 1′-(2-chlorobenzoyl)-2-oxospiro[indoline-3,4′-piperidine]-5-carboxylate

To a suspension of methyl 2-oxospiro[indoline-3,4′-piperidine]-5-carboxylate hydrochloride (50.0 mg, 0.169 mmol), 2-chlorobenzoic acid (29.0 mg, 0.185 mmol) and HBTU (70.3 mg, 0.185 mmol) in chloroform (5 mL) was added DIPEA (90.2 μL, 0.506 mmol). The resulting mixture was stirred at room temperature for 18 h. The solvent was removed with a stream of N₂ and the residue triturated with water/MeOH (5:1) for 18 h. The solid was collected by filtration, washed with water and dried to give methyl 1′-(2-chlorobenzoyl)-2-oxospiro[indoline-3,4′-piperidine]-5-carboxylate (50.0 mg, 0.125 mmol, 74.4% yield) as a white solid.

¹H NMR (400 MHz, DMSO-d₆) δ ppm 10.87-10.95 (m, 1H) 7.65-8.09 (m, 3H) 7.52-7.61 (m, 1H) 7.42-7.50 (m, 2H) 6.93-7.02 (m, 1H) 4.13-4.35 (m, 1H) 3.83 (s, 3H) 3.61-3.79 (m, 2H) 3.23 (d, J=13.39 Hz, 1H) 1.60-2.10 (m, 5H)

LCMS: [M+1]=399/401, rt=2.27 min.

### 1′-(2-chlorobenzoyl)-2-oxospiro[indoline-3,4′-piperidine]-5-carboxylic acid

To a solution of methyl 1′-(2-chlorobenzoyl)-2-oxospiro[indoline-3,4′-piperidine]-5-carboxylate (50.0 mg, 0.125 mmol) in methanol (8 mL) was added 1 N NaOH (376 μL, 0.376 mmol). The resulting mixture was stirred at 50° C. for 48 h. The solvent was removed with a stream of N₂ and the residue was taken up in water, filtered through a syringe filter and acidified by addition of 6 N HCl. After stirring for 18 h, the solid was collected by filtration and dried to give a beige solid which was further purified by automated normal-phase chromatography (0-40% MeOH/DCM, 20 g silica gel cartridge) to give 1′-(2-chlorobenzoyl)-2-oxospiro[indoline-3,4′-piperidine]-5-carboxylic acid (38.8 mg, 0.101 mmol, 80.4% yield) as an off-white solid.

¹H NMR (400 MHz, DMSO-d₆) δ ppm 12.74 (br. s., 1H) 10.83-10.89 (m, 1H) 7.81-8.06 (m, 2H) 7.42-7.70 (m, 4H) 6.92-6.98 (m, 1H) 4.13-4.29 (m, 1H) 3.61-3.93 (m, 2H) 3.19-3.32 (m, 1H) 1.62-2.05 (m, 4H)

LCMS: [M+1]=385, rt=1.95 min.

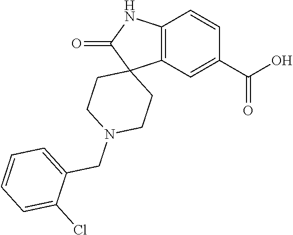 [SMILES: O=C(O)c1ccc2c(c1)C1(CCN(Cc3ccccc3Cl)CC1)C(=O)N2]

### Example 15: 1′-(2-chlorobenzyl)-2-oxospiro[indoline-3,4′-piperidine]-5-carboxylic acid

### Methyl 1′-(2-chlorobenzyl)-2-oxospiro[indoline-3,4′-piperidine]-5-carboxylate

A suspension of methyl 2-oxospiro[indoline-3,4′-piperidine]-5-carboxylate hydrochloride (47.0 mg, 0.158 mmol), 2-chlorobenzaldehyde (19.6 μL, 0.174 mmol) and sodium triacetoxyborohydride (101 mg, 0.475 mmol) in CHCl₃ (5 mL) was stirred at room temperature for 5 days (for convenience) The contents were treated with 5% Na₂CO₃ and extracted with CHCl₃ (3×) and passed through a cotton plug. The solvent was removed with a stream of N₂ to give a residue which was purified by automated normal-phase chromatography (0-20% MeOH/DCM, 4 g silica gel cartridge) to give methyl 1′-[(2-chlorophenyl)methyl]-2-oxospiro[indoline-3,4′-piperidine]-5-carboxylate (46.3 mg, 0.120 mmol, 76.0% yield) as a white solid.

¹H NMR (400 MHz, DMSO-d₆) δ ppm 10.83 (s, 1H) 7.98 (d, J=1.26 Hz, 1H) 7.88 (dd, J=8.08, 1.77 Hz, 1H) 7.60 (dd, J=7.58, 1.77 Hz, 1H) 7.46 (dd, J=7.83, 1.26 Hz, 1H) 7.37 (td, J=7.45, 1.52 Hz, 1H) 7.28-7.34 (m, 1H) 6.97 (d, J=8.08 Hz, 1 H) 3.83 (s, 3H) 3.72 (s, 2H) 2.91 (ddd, J=11.49, 7.58, 3.66 Hz, 2H) 2.59-2.69 (m, 2H) 1.80-1.88 (m, 2H) 1.70-1.79 (m, 2H)

LCMS: [M+1]=385/387, rt=1.91 min.

### 1′-(2-chlorobenzyl)-2-oxospiro[indoline-3,4′-piperidine]-5-carboxylic acid

To a suspension of methyl 1′-[(2-chlorophenyl)methyl]-2-oxospiro[indoline-3,4′-piperidine]-5-carboxylate (46.3 mg, 0.120 mmol) in methanol (5 mL) was added 1 N NaOH (602 μL, 0.602 mmol). The resulting mixture was stirred at 60° C. for 22 h.

After cooling, the solvent was removed under a stream of N₂, and the residue taken up in water and filtered through a syringe filter. The solution was carefully neutralized by addition of 2 N HCl and stirred for 3 h. The solid was collected by filtration, washed with water and dried under vacuum to give 1′-[(2-chlorophenyl)methyl]-2-oxospiro[indoline-3,4′-piperidine]-5-carboxylic acid (3.2 mg, 0.0086 mmol, 7.2% yield) as an off-white solid.

¹H NMR (400 MHz, DMSO-d₆) δ ppm 12.71 (br. s., 1H) 10.78 (br. s., 1H) 8.00 (br. s., 1H) 7.86 (d, J=6.82 Hz, 1H) 7.62 (br. s., 1H) 7.45 (d, J=7.58 Hz, 1H) 7.27-7.40 (m, 2H) 6.95 (d, J=7.33 Hz, 1H) 3.72 (br. s., 2H) 2.91 (br. s., 2H) 2.64 (br. s., 2H) 1.66-1.90 (m, 4H)

LCMS: [M+1]=371/373, rt=1.69 min.

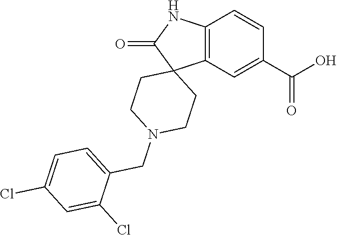 [SMILES: O=C(O)c1ccc2c(c1)C1(CCN(Cc3ccc(Cl)cc3Cl)CC1)C(=O)N2]

### Example 16: 1′-(2,4-dichlorobenzyl)-2-oxospiro[indoline-3,4′-piperidine]-5-carboxylic acid

### Methyl 1′-(2,4-dichlorobenzyl)-2-oxospiro[indoline-3,4′-piperidine]-5-carboxylate

A suspension of methyl 2-oxospiro[indoline-3,4′-piperidine]-5-carboxylate hydrochloride (41.4 mg, 0.140 mmol), 2,4-dichlorobenzaldehyde (26.9 mg, 0.154 mmol) and sodium triacetoxyborohydride (88.7 mg, 0.419 mmol) in CHCl₃ (5 mL) was stirred at room temperature for 4 days (for convenience). The contents were treated with 5% Na₂CO₃ and extracted with CHCl₃ (3×) and passed through a cotton plug. The solvent was removed with a stream of N₂ to give a residue which was purified by automated normal-phase chromatography (0-20% MeOH/DCM, 4 g silica gel cartridge) to give methyl 1′-[(2,4-dichlorophenyl)methyl]-2-oxospiro[indoline-3,4′-piperidine]-5-carboxylate (44.8 mg, 0.107 mmol, 76.6% yield) as a white solid.

¹H NMR (400 MHz, DMSO-d₆) δ ppm 10.83 (s, 1H) 7.98 (d, J=1.52 Hz, 1H) 7.88 (dd, J=8.08, 1.52 Hz, 1H) 7.62-7.65 (m, 2H) 7.45 (dd, J=8.34, 2.27 Hz, 1H) 6.97 (d, J=8.08 Hz, 1H) 3.83 (s, 3H) 3.70 (s, 2H) 2.90 (td, J=7.58, 3.54 Hz, 2H) 2.63 (dt, J=7.39, 3.76 Hz, 2H) 1.80-1.87 (m, 2H) 1.71-1.79 (m, 2H)

LCMS: [M+1]=419/421/423, rt=2.07 min.

### 1′-(2,4-dichlorobenzyl)-2-oxospiro[indoline-3,4′-piperidine]-5-carboxylic acid

To a suspension of methyl 1′-[(2,4-dichlorophenyl)methyl]-2-oxospiro[indoline-3,4′-piperidine]-5-carboxylate (44.8 mg, 0.107 mmol) in methanol (5 mL) was added 1 N NaOH (534 μL, 0.534 mmol). The resulting mixtures was stirred at 50° C. for 22 h. After cooling, the solvent was removed under a stream of N₂, and the residue taken up in water and filtered through a syringe filter. The solution was carefully neutralized by addition of 2 N HCl and stirred for 3 h. The solid was collected by filtration, washed with water and dried under vacuum to give 1′-[(2,4-dichlorophenyl)methyl]-2-oxospiro[indoline-3,4′-piperidine]-5-carboxylic acid (38.0 mg, 0.0938 mmol, 87.8% yield) as a white solid.

¹H NMR (400 MHz, DMSO-d₆) δ ppm 12.72 (br. s., 1H) 10.79 (s, 1H) 7.99 (s, 1H) 7.86 (dd, J=8.08, 1.52 Hz, 1H) 7.61-7.66 (m, 2H) 7.45 (dd, J=8.34, 2.27 Hz, 1H) 6.94 (d, J=8.08 Hz, 1H) 3.70 (s, 2H) 2.89 (d, J=7.83 Hz, 2H) 2.63 (br. s., 2H) 1.83 (d, J=8.08 Hz, 2H) 1.74 (br. s., 2H)

LCMS: [M+1]=405/407/409, rt=1.88 min.

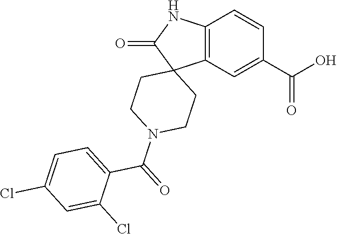 [SMILES: O=C(O)c1ccc2c(c1)C1(CCN(C(=O)c3ccc(Cl)cc3Cl)CC1)C(=O)N2]

### Example 17: 1′-(2,4-dichlorobenzoyl)-2-oxospiro[indoline-3,4′-piperidine]-5-carboxylic acid

### Methyl 1′-(2,4-dichlorobenzoyl)-2-oxospiro[indoline-3,4′-piperidine]-5-carboxylate

To a suspension of methyl 2-oxospiro[indoline-3,4′-piperidine]-5-carboxylate hydrochloride (41.0 mg, 0.138 mmol), 2,4-dichlorobenzoic acid (29.0 mg, 0.152 mmol) and HBTU (57.6 mg, 0.152 mmol) in CHCl₃ (5 mL) was added DIPEA (74.0 μL, 0.415 mmol). The resulting mixture was stirred at room temperature for 7 days (for convenience). The contents were treated with water and extracted with CHCl₃ (3×). The organic layers were filtered through a cotton plug, reduced in volume with a stream of N₂ and then purified by automated normal-phase chromatography (0-20% MeOH/DCM, 4 g silica gel cartridge) to give methyl 1′-(2,4-dichlorobenzoyl)-2-oxospiro[indoline-3,4′-piperidine]-5-carboxylate (50.5 mg, 0.117 mmol, 84.4% yield) as an off white solid.

¹H NMR (400 MHz, DMSO-d₆) δ ppm 10.85-10.91 (m, 1H) 7.47-8.08 (m, 5H) 6.94-7.00 (m, 1H) 4.13-4.33 (m, 1H) 3.63-3.89 (m, 5H) 3.19-3.29 (m, 1H) 1.59-2.10 (m, 4H)

LCMS: [M+1]=433/435/437, rt=2.53 min.

### 1′-(2,4-dichlorobenzoyl)-2-oxospiro[indoline-3,4′-piperidine]-5-carboxylic acid

To a suspension of methyl 1′-(2,4-dichlorobenzoyl)-2-oxospiro[indoline-3,4′-piperidine]-5-carboxylate (50.5 mg, 0.117 mmol) in methanol (5 mL) was added 1 N NaOH (583 μL, 0.583 mmol). The resulting mixture was stirred at 50° C. for 24 h. The solvent was removed under a stream of N₂, and the residue taken up in water and filtered through a syringe filter. The solution was acidified by addition of 6 N HCl and stirred for 3 days. The solid was collected by filtration, washed with water and dried under vacuum to give 1′-(2,4-dichlorobenzoyl)-2-oxospiro[indoline-3,4′-piperidine]-5-carboxylic acid (33.8 mg, 0.0806 mmol, 69.2% yield) as a white solid.

¹H NMR (400 MHz, DMSO-d₆) δ ppm 12.70 (br. s., 1H) 10.79-10.87 (m, 1H) 7.46-8.06 (m, 5H) 6.90-6.98 (m, 1H) 4.11-4.29 (m, 1H) 3.62-3.92 (m, 2H) 3.20-3.29 (m, 1H) 1.60-2.06 (m, 4H)

LCMS: [M+1]=419/421/423, rt=2.18 min.

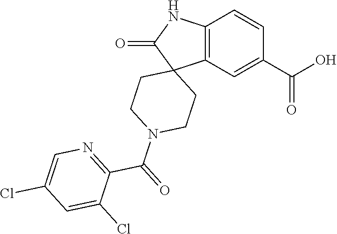 [SMILES: O=C(O)c1ccc2c(c1)C1(CCN(C(=O)c3ncc(Cl)cc3Cl)CC1)C(=O)N2]

### Example 18: 1′-((3,5-dichloropyridin-2-yl)methyl)-2-oxospiro[indoline-3,4′-piperidine]-5-carboxylic acid

### Methyl 1′-((3,5-dichloropyridin-2-yl)methyl)-2-oxospiro[indoline-3,4′-piperidine]-5-carboxylate

A mixture of methyl 2-oxospiro[indoline-3,4′-piperidine]-5-carboxylate hydrochloride (48.8 mg, 0.164 mmol), 3,5-dichloropyridine-2-carboxaldehyde (17.5 μL, 0.181 mmol) and sodium triacetoxyborohydride (104.6 mg, 0.493 mmol) in CHCl₃ (5 mL) was stirred at room temperature for 6 days (for convenience) The contents were treated with 10% Na₂CO₃ and extracted with CHCl₃ (3×) and passed through a cotton plug. The solvent was removed with a stream of N₂ to give a residue which was purified by automated normal-phase chromatography (0-20% MeOH/DCM, 4 g silica gel cartridge) to give methyl 1′-[(3,5-dichloro-2-pyridyl)methyl]-2-oxospiro[indoline-3,4′-piperidine]-5-carboxylate (25.0 mg, 0.0595 mmol, 36.2% yield) as an off-white solid.

¹H NMR (400 MHz, DMSO-d₆) δ ppm 10.80 (s, 1H) 8.63 (d, J=2.20 Hz, 1H) 8.25 (d, J=2.20 Hz, 1H) 7.98 (s, 1H) 7.87 (dd, J=8.17, 1.73 Hz, 1H) 6.96 (d, J=8.17 Hz, 1H) 3.87 (s, 2H) 3.83 (s, 3H) 2.90-2.96 (m, 2H) 2.72-2.80 (m, 2H) 1.76-1.84 (m, 2H) 1.61-1.69 (m, 2H)

LCMS: [M+1]=434/436/438, rt=1.94 min.

### 1′-((3,5-dichloropyridin-2-yl)methyl)-2-oxospiro[indoline-3,4′-piperidine]-5-carboxylic acid

To a suspension of methyl 1′-[(3,5-dichloro-2-pyridyl)methyl]-2-oxospiro[indoline-3,4′-piperidine]-5-carboxylate (25.0 mg, 0.0595 mmol) in methanol (5 mL) was added 1 N NaOH (297 μL, 0.297 mmol). The resulting mixture was stirred at 60° C. for 24 h. The solvent was removed under a stream of N₂, and the residue taken up in water and filtered through a syringe filter. The solution was acidified by addition of 6 N HCl and stirred for 18 h. The solid was collected by filtration, washed with water and dried under vacuum to give 1′-[(3,5-dichloro-2-pyridyl)methyl]-2-oxospiro[indoline-3,4′-piperidine]-5-carboxylic acid hydrochloride (12.1 mg, 0.0273 mmol, 45.948% yield) as a white solid.

¹H NMR (400 MHz, DMSO-d₆) δ ppm 12.77 (br. s., 1H) 10.90-11.10 (m, 1H) 10.08-10.52 (m, 1H) 8.76-8.89 (m, 1H) 8.36-8.49 (m, 2H) 7.86-7.96 (m, 1H) 7.81 (s, 1H) 6.94-7.05 (m, 1H) 4.77-5.03 (m, 2H) 3.55-3.86 (m, 4H) 1.92-2.30 (m, 2H)

LCMS: [M+1]=406/408/410, rt=1.71 min.

### Synthesis of Representative Benzimidazolones of Formula (IB)

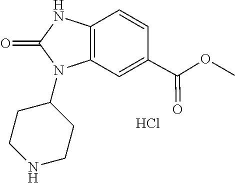 [SMILES: COC(=O)c1ccc2[nH]c(=O)n(C3CCNCC3)c2c1 ; Cl]

### Intermediate A: Methyl 2-oxo-3-(piperidin-4-yl)-2,3-dihydro-1H-benzo[d]imidazole-5-carboxylate hydrochloride

### tert-butyl 4-((5-(methoxycarbonyl)-2-nitrophenyl)amino)piperidine-1-carboxylate

A mixture of methyl 3-fluoro-4-nitrobenzoate (302 mg, 1.52 mmol), tert-butyl 4-aminopiperidine-1-carboxylate (364 mg, 1.82 mmol) and cesium carbonate (593 mg, 1.82 mmol) in acetonitrile (10 mL) was stirred at room temperature for 90 h. The contents were taken up in EtOAc, washed with water (3×), brine (lx), dried over MgSO₄, filtered and the solvent removed in vacuo to give a residue which was purified by automated normal-phase chromatography (0-100% EtOAc/heptane, 20 g silica gel cartridge) to give tert-butyl 4-((5-(methoxycarbonyl)-2-nitrophenyl)amino)piperidine-1-carboxylate (440 mg, 1.16 mmol, 76.5% yield) as a yellow-orange solid.

¹H NMR (400 MHz, DMSO-d₆) δ ppm 8.19 (d, J=8.84 Hz, 1H) 7.90 (d, J=8.08 Hz, 1H) 7.60 (d, J=1.52 Hz, 1H) 7.17 (dd, J=8.84, 1.77 Hz, 1H) 3.84-3.98 (m, 6H) 3.02 (br. s., 2H) 1.94 (d, J=10.36 Hz, 2H) 1.44-1.55 (m, 2H) 1.42 (s, 9H)

LCMS: [M−1+23]=402, rt=1.90 min (lipophilic method).

### tert-butyl 4-((2-amino-5-(methoxycarbonyl)phenyl)amino)piperidine-1-carboxylate

To a solution of tert-butyl 4-((5-(methoxycarbonyl)-2-nitrophenyl)amino)piperidine-1-carboxylate (440 mg, 1.16 mmol) in EtOAc (20 mL) was added 10% Pd/C (40 mg). The resulting suspension was placed under an atmosphere of H₂ via a balloon and stirred at room temperature for 18 h. The contents were filtered through Celite and the solvent removed in vacuo to give tert-butyl 4-((2-amino-5-(methoxycarbonyl)phenyl)amino)piperidine-1-carboxylate (400 mg, 1.145 mmol, 98.7% yield) as an off-white solid.

¹H NMR (400 MHz, DMSO-d₆) δ ppm 7.14 (dd, J=8.08, 1.77 Hz, 1H) 7.04 (d, J 1.77 Hz, 1H) 6.55 (d, J=8.08 Hz, 1H) 5.42-5.48 (m, 2H) 4.41 (d, J=7.58 Hz, 1H) 3.89 (d, J=10.11 Hz, 2H) 3.72 (s, 3H) 3.39-3.50 (m, 1H) 2.94 (br. s., 2H) 1.91 (d, J=10.11 Hz, 2H) 1.41 (s, 9H) 1.21-1.33

LCMS: [M−1+23]=350, rt=1.14 min (lipophilic method).

### Methyl 3-(1-(tert-butoxycarbonyl)piperidin-4-yl)-2-oxo-2,3-dihydro-1H-benzo[d]imidazole-5-carboxylate

To a solution of tert-butyl 4-((2-amino-5-(methoxycarbonyl)phenyl)amino)piperidine-1-carboxylate (247 mg, 0.708 mmol) in chloroform (5 mL) was added 1,1′-carbonyldiimidazole (172 mg, 1.06 mmol). The resulting mixture was stirred at 50° C. for 18 h. Added 1,1′-carbonyldiimidazole (86 mg, 0.53 mmol) and stirred at 50° C. for 18 h. The solvent was removed under a stream of N₂, and the residue was purified by automated normal-phase chromatography (0-100% EtOAc/heptane, 4 g silica gel cartridge) to give methyl 3-(1-(tert-butoxycarbonyl)piperidin-4-yl)-2-oxo-2,3-dihydro-1H-benzo[d]imidazole-5-carboxylate (220 mg, 0.586 mmol, 82.8% yield) as an off-white solid.

¹H NMR (400 MHz, DMSO-d₆) δ ppm 11.36 (s, 1H) 7.72 (d, J=1.26 Hz, 1H) 7.69 (dd, J=8.08, 1.52 Hz, 1H) 7.09 (d, J=8.08 Hz, 1H) 4.39-4.49 (m, 1H) 4.01-4.14 (m, 2H) 3.83 (s, 3H) 2.91 (br. s., 2H) 2.11-2.24 (m, 2H) 1.71 (d, J=10.11 Hz, 2H) 1.45 (s, 9H)

LCMS: [M−1]+23=398, rt=2.44 min

### Methyl 2-oxo-3-(piperidin-4-yl)-2,3-dihydro-1H-benzo[d]imidazole-5-carboxylate hydrochloride

To a suspension of methyl 3-(1-(tert-butoxycarbonyl)piperidin-4-yl)-2-oxo-2,3-dihydro-1H-benzo[d]imidazole-5-carboxylate (220 mg, 0.586 mmol) in methanol (5 mL) was added 6 N HCl (977 μL, 5.86 mmol). The resulting mixture was stirred at 60° C. for 4 h. The solvent was removed in vacuo to give methyl 2-oxo-3-(piperidin-4-yl)-2,3-dihydro-1H-benzo[d]imidazole-5-carboxylate hydrochloride (188 mg, 0.605 mmol, 103% yield) as a beige solid.

¹H NMR (400 MHz, DMSO-d₆) δ ppm 11.43 (s, 1H) 9.03 (d, J=11.37 Hz, 1H) 8.67 (d, J=8.59 Hz, 1H) 7.90 (s, 1H) 7.71 (dd, J=8.08, 1.52 Hz, 1H) 7.11 (d, J=8.08 Hz, 1H) 4.59 (ddd, J=12.13, 8.08, 3.79 Hz, 1H) 3.85 (s, 3H) 3.42 (d, J=11.87 Hz, 2H) 3.05-3.15 (m, 2H) 2.53-2.64 (m, 2H) 1.89 (d, J=11.87 Hz, 2H)

LCMS: [M+1]=276, rt=1.46 min

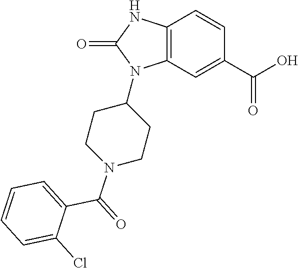 [SMILES: O=C(O)c1ccc2[nH]c(=O)n(C3CCN(C(=O)c4ccccc4Cl)CC3)c2c1]

### Example 1: 3-(1-(2-chlorobenzoyl)piperidin-4-yl)-2-oxo-2,3-dihydro-1H-benzo[d]imidazole-5-carboxylic acid

### Methyl 3-(1-(2-chlorobenzoyl)piperidin-4-yl)-2-oxo-2,3-dihydro-1H-benzo[d]imidazole-5-carboxylate

A mixture of methyl 2-oxo-3-(piperidin-4-yl)-2,3-dihydro-1H-benzo[d]imidazole-5-carboxylate hydrochloride (30.0 mg, 0.0962 mmol), 2-chlorobenzoic acid (15.1 mg, 0.0962 mmol), HBTU (36.5 mg, 0.0962 mmol) and DIPEA (17.2 μL, 0.0962 mmol) in chloroform (5 mL) was stirred at room temperature for 41 h. The solvent was removed with a stream of N₂ and the residue was purified by automated normal-phase chromatography (0-100% EtOAc/heptane, 4 g silica gel cartridge) to give methyl 3-(1-(2-chlorobenzoyl)piperidin-4-yl)-2-oxo-2,3-dihydro-1H-benzo[d]imidazole-5-carboxylate (30.1 mg, 0.0727 mmol, 75.6% yield) as an off-white solid. Two amide isomers seen by 1H NMR—one peak by LCMS.

¹H NMR (400 MHz, DMSO-d₆) δ ppm 11.35-11.40 (m, 1H) 7.75-7.83 (m, 1H) 7.68-7.72 (m, 1H) 7.54-7.62 (m, 1H) 7.37-7.50 (m, 3H) 7.06-7.12 (m, 1H) 4.52-4.76 (m, 2H) 3.83-3.87 (m, 3H) 3.57-3.68 (m, 1H) 3.22-3.31 (m, 1H) 3.10-3.19 (m, 1H) 2.94-3.05 (m, 1H) 2.14-2.43 (m, 3H) 1.87 (d, J=10.36 Hz, 1H) 1.64-1.75 (m, 1H)

LCMS: [M+1]=414/416, rt=2.20 min.

### 3-(1-(2-chlorobenzoyl)piperidin-4-yl)-2-oxo-2,3-dihydro-1H-benzo[d]imidazole-5-carboxylic acid

To a solution of methyl 3-(1-(2-chlorobenzoyl)piperidin-4-yl)-2-oxo-2,3-dihydro-1H-benzo[d]imidazole-5-carboxylate (29.0 mg, 0.0701 mmol) in methanol (5 mL) was added 1 N NaOH (210 μL, 0.210 mmol). The resulting mixture was stirred at 60° C. for 72 h. After cooling, the solvent was removed under a stream of N₂, and the residue taken up in water and filtered through a syringe filter. The solution was acidified by addition of 6 N HCl and stirred for 3 h. The solid was collected by filtration, washed with water and dried under vacuum to give 3-(1-(2-chlorobenzoyl)piperidin-4-yl)-2-oxo-2,3-dihydro-1H-benzo[d]imidazole-5-carboxylic acid (12.9 mg, 0.0323 mmol, 46.0% yield) as an off-white solid. Two amide conformers seen by ¹H NMR—one peak by LCMS.

¹H NMR (400 MHz, DMSO-d₆) δ ppm 12.79 (br. s., 1H) 11.30-11.33 (m, 1H) 7.75-7.82 (m, 1H) 7.67 (dd, J=8.21, 1.39 Hz, 1H) 7.53-7.60 (m, 1H) 7.35-7.50 (m, 3H) 7.04-7.09 (m, 1H) 4.64-4.76 (m, 1H) 4.51-4.64 (m, 1H) 3.21-3.39 (m, 2H) 2.93-3.04 (m, 1H) 2.15-2.43 (m, 2H) 1.87 (d, J=11.62 Hz, 1H) 1.69 (br. s., 1H)

LCMS: [M+1]=400/402, rt=1.88 min.

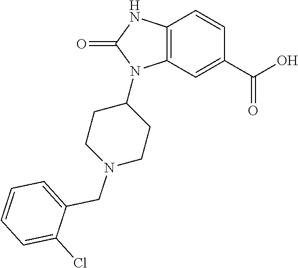 [SMILES: O=C(O)c1ccc2[nH]c(=O)n(C3CCN(Cc4ccccc4Cl)CC3)c2c1]

### Example 2: 3-(1-(2-chlorobenzyl)piperidin-4-yl)-2-oxo-2,3-dihydro-1H-benzo[d]imidazole-5-carboxylic acid

### Methyl 3-(1-(2-chlorobenzyl)piperidin-4-yl)-2-oxo-2,3-dihydro-1H-benzo[d]imidazole-5-carboxylate

A mixture of methyl 2-oxo-3-(piperidin-4-yl)-2,3-dihydro-1H-benzo[d]imidazole-5-carboxylate hydrochloride (41.0 mg, 0.132 mmol), 2-chlorobenzaldehyde (16.3 μL, 0.145 mmol) and sodium triacetoxyborohydride (83.6 mg, 0.395 mmol) in chloroform (5 mL) was stirred at room temperature for 4 days (for convenience). The contents were treated with 5% Na₂CO₃ and extracted with CHCl₃ (3×) and passed through a cotton plug. The solvent was removed with a stream of N₂ to give a residue which was purified by automated normal-phase chromatography (0-20% MeOH/DCM, 4 g silica gel cartridge) to give methyl 3-(1-(2-chlorobenzyl)piperidin-4-yl)-2-oxo-2,3-dihydro-1H-benzo[d]imidazole-5-carboxylate (42.4 mg, 0.106 mmol, 80.6% yield) as a white solid.

¹H NMR (400 MHz, DMSO-d₆) δ ppm 11.35 (s, 1H) 7.76 (d, J=1.26 Hz, 1H) 7.69 (dd, J=8.08, 1.52 Hz, 1H) 7.56 (dd, J=7.58, 1.77 Hz, 1H) 7.46 (dd, J=7.71, 1.39 Hz, 1H) 7.38 (td, J=7.39, 1.39 Hz, 1H) 7.28-7.34 (m, 1H) 7.09 (d, J=8.34 Hz, 1H) 4.19-4.29 (m, 1H) 3.85 (s, 3H) 3.64 (s, 2H) 2.98 (d, J=10.61 Hz, 2H) 2.31-2.43 (m, 2H) 2.21-2.30 (m, 2H) 1.69 (d, J=11.62 Hz, 2H)

LCMS: [M+1]=400/402, rt=1.91 min.

### 3-(1-(2-chlorobenzyl)piperidin-4-yl)-2-oxo-2,3-dihydro-1H-benzo[d]imidazole-5-carboxylic acid

To a suspension of methyl 3-(1-(2-chlorobenzyl)piperidin-4-yl)-2-oxo-2,3-dihydro-1H-benzo[d]imidazole-5-carboxylate (42.4 mg, 0.106 mmol) in methanol (5 mL) was added 10% NaOH (382 μL, 1.06 mmol). The resulting mixture was stirred at 50° C. for 42 h. The solvent was removed with a stream of N₂ and the residue dissolved in water. The solution was acidified first by addition of 6 N HCl (177 μL, 1.06 mmol), then by careful dropwise addition of 6 N HCl. Once a precipitate appeared the mixture was allowed to stir for 2 h as the amount of precipitate increased. The solid was collected by filtration then dried under vacuum to give 3-(1-(2-chlorobenzyl)piperidin-4-yl)-2-oxo-2,3-dihydro-1H-benzo[d]imidazole-5-carboxylic acid hydrochloride (33.2 mg, 0.0786 mmol, 74.1% yield) as a white solid.

¹H NMR (400 MHz, DMSO-d₆) δ ppm 12.79 (br. s., 1H) 11.40 (s, 1H) 10.26 (br. s., 1H) 7.86 (br. s., 2H) 7.69 (d, J=8.08 Hz, 1H) 7.64 (d, J=7.33 Hz, 1H) 7.48-7.58 (m, 2H) 7.08 (d, J=8.08 Hz, 1H) 4.55-4.66 (m, 1H) 4.50 (d, J=4.29 Hz, 2H) 3.56 (d, J=11.87 Hz, 2H) 3.33-3.44 (m, 2H) 2.70 (d, J=13.39 Hz, 2H) 1.95 (d, J 11.12 Hz, 2H)

LCMS: [M+1]=386/388, rt=1.66 min.

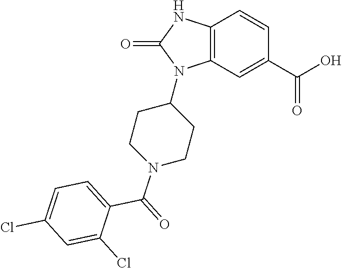 [SMILES: O=C(O)c1ccc2[nH]c(=O)n(C3CCN(C(=O)c4ccc(Cl)cc4Cl)CC3)c2c1]

### Example 3: 3-(1-(2,4-dichlorobenzoyl)piperidin-4-yl)-2-oxo-2,3-dihydro-1H-benzo[d]imidazole-5-carboxylic acid

### Methyl 3-(1-(2,4-dichlorobenzoyl)piperidin-4-yl)-2-oxo-2,3-dihydro-1H-benzo[d]imidazole-5-carboxylate

A mixture of methyl 2-oxo-3-(piperidin-4-yl)-2,3-dihydro-1H-benzo[d]imidazole-5-carboxylate hydrochloride (36.4 mg, 0.117 mmol), 2,4-dichlorobenzoic acid (22.3 mg, 0.117 mmol), HBTU (44.3 mg, 0.117 mmol) and DIPEA (20.8 μL, 0.117 mmol) in chloroform (5 mL) was stirred at room temperature for 42 h. The solvent was removed with a stream of N₂ and the residue which was purified by automated normal-phase chromatography (0-100% EtOAc/heptane, 4 g silica gel cartridge) to give methyl 3-(1-(2,4-dichlorobenzoyl)piperidin-4-yl)-2-oxo-2,3-dihydro-1H-benzo[d]imidazole-5-carboxylate (45.0 mg, 0.100 mmol, 86.0% yield) as a colorless gum. Two amide isomers seen by 1H NMR—one peak by LCMS

¹H NMR (400 MHz, DMSO-d₆) δ ppm 11.32-11.37 (m, 1H) 7.73-7.82 (m, 2H) 7.70 (dd, J=8.17, 1.26 Hz, 1H) 7.52-7.59 (m, 1H) 7.41-7.50 (m, 1H) 7.06-7.12 (m, 1H) 4.52-4.73 (m, 2H) 3.83-3.87 (m, 3H) 3.23-3.40 (m, 2H) 2.94-3.04 (m, 1H) 2.15-2.39 (m, 2H) 1.87 (d, J=12.42 Hz, 1H) 1.65-1.75 (m, 1H)

LCMS: [M+1]=448/450/452, rt=2.43 min.

### 3-(1-(2,4-dichlorobenzoyl)piperidin-4-yl)-2-oxo-2,3-dihydro-1H-benzo[d]imidazole-5-carboxylic acid

To a solution of methyl 3-(1-(2,4-dichlorobenzoyl)piperidin-4-yl)-2-oxo-2,3-dihydro-1H-benzo[d]imidazole-5-carboxylate (45.0 mg, 0.100 mmol) in methanol (5 mL) was added 1 N NaOH (502 μL, 0.502 mmol). The resulting mixture was stirred at 60° C. for 51 h. After cooling, the solvent was removed under a stream of N₂, and the residue taken up in water and filtered through a syringe filter. The solution was acidified by addition of 6 N HCl and stirred for 18 h. The solid was collected by filtration, washed with water and dried under vacuum to give 3-(1-(2,4-dichlorobenzoyl)piperidin-4-yl)-2-oxo-2,3-dihydro-1H-benzo[d]imidazole-5-carboxylic acid (25.7 mg, 0.0592 mmol, 59.0% yield) as a white solid. Two amide isomers seen by 1H NMR—one peak by LCMS.

¹H NMR (400 MHz, DMSO-d₆) δ ppm 12.71 (s, 1H) 11.26-11.30 (m, 1H) 7.40-7.82 (m, 5H) 7.03-7.09 (m, 1H) 4.63-4.74 (m, 1H) 4.50-4.63 (m, 1H) 3.22-3.40 (m, 2H) 2.94-3.04 (m, 1H) 2.15-2.38 (m, 2H) 1.87 (d, J=11.63 Hz, 1H) 1.71 (d, J=11.16 Hz, 1H)

LCMS: [M+1]=434/436/438, rt=2.09 min.

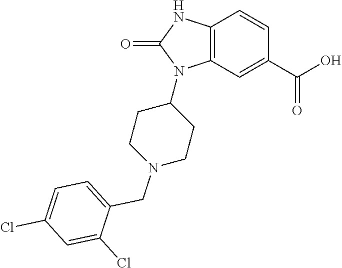 [SMILES: O=C(O)c1ccc2[nH]c(=O)n(C3CCN(Cc4ccc(Cl)cc4Cl)CC3)c2c1]

### Example 4: 3-(1-(2,4-dichlorobenzyl)piperidin-4-yl)-2-oxo-2,3-dihydro-1H-benzo[d]imidazole-5-carboxylic acid

### Methyl 3-(1-(2,4-dichlorobenzoyl)piperidin-4-yl)-2-oxo-2,3-dihydro-1H-benzo[d]imidazole-5-carboxylate

A mixture of methyl 2-oxo-3-(piperidin-4-yl)-2,3-dihydro-1H-benzo[d]imidazole-5-carboxylate hydrochloride (35.3 mg, 0.113 mmol), 2,4-dichlorobenzaldehyde (17.5 μL, 0.125 mmol) and Sodium triacetoxyborohydride (72.0 mg, 0.340 mmol) in chloroform (5 mL) was stirred at room temperature for 3 days (for convenience) The contents were treated with 5% Na₂CO₃ and extracted with CHCl₃ (3×) and passed through a cotton plug. The solvent was removed with a stream of N₂ to give a residue which was purified by automated normal-phase chromatography (0-20% MeOH/DCM, 4 g silica gel cartridge) to give methyl 3-(1-(2,4-dichlorobenzoyl)piperidin-4-yl)-2-oxo-2,3-dihydro-1H-benzo[d]imidazole-5-carboxylate (22.9 mg, 0.0527 mmol, 46.6% yield) as a white solid.

¹H NMR (400 MHz, DMSO-d₆) δ ppm 11.32 (br. s., 1H) 7.75 (d, J=1.10 Hz, 1H) 7.68 (dd, J=8.17, 1.41 Hz, 1H) 7.61 (d, J=2.04 Hz, 1H) 7.57 (d, J=8.33 Hz, 1H) 7.47 (dd, J=8.25, 2.12 Hz, 1H) 7.08 (d, J=8.17 Hz, 1H) 4.19-4.27 (m, 1H) 3.85 (s, 3H) 3.62 (s, 2H) 2.96 (d, J=11.00 Hz, 2H) 2.32-2.42 (m, 2H) 2.22-2.30 (m, 2H) 1.70 (d, J=12.73 Hz, 2H)

LCMS: [M+1]=434/436/438, rt=2.07 min.

### 3-(1-(2,4-dichlorobenzyl)piperidin-4-yl)-2-oxo-2,3-dihydro-1H-benzo[d]imidazole-5-carboxylic acid

To a suspension of methyl 3-(1-(2,4-dichlorobenzoyl)piperidin-4-yl)-2-oxo-2,3-dihydro-1H-benzo[d]imidazole-5-carboxylate (22.9 mg, 0.0527 mmol) in methanol (5 mL) was added 1 N NaOH (264 μL, 0.264 mmol). the resulting mixture was stirred at 60° C. for 58 h. The solvent was removed under a stream of N₂, and the residue taken up in water and filtered through a syringe filter. The solution was acidified by addition of 6 N HCl and stirred for 18 h. The solid was collected by filtration, washed with water and dried under vacuum to give 3-(1-(2,4-dichlorobenzyl)piperidin-4-yl)-2-oxo-2,3-dihydro-1H-benzo[d]imidazole-5-carboxylic acid hydrochloride (16.6 mg, 0.0363 mmol, 68.9% yield) as a white solid.

¹H NMR (400 MHz, DMSO-d₆) δ ppm 12.75 (br. s., 1H) 11.35 (s, 1H) 10.14 (br. s., 1H) 7.81-7.87 (m, 3H) 7.62-7.71 (m, 2H) 7.07 (d, J=8.02 Hz, 1H) 4.58 (t, J=12.03 Hz, 1H) 4.48 (d, J=4.24 Hz, 2H) 3.57 (d, J=11.00 Hz, 2H) 2.61-2.74 (m, 2H) 1.95 (d, J=11.95 Hz, 2H)

LCMS: [M+1]=420/422/424, rt=1.85 min.

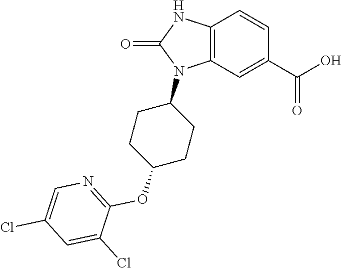 [SMILES: O=C(O)c1ccc2[nH]c(=O)n([C@H]3CC[C@H](Oc4ncc(Cl)cc4Cl)CC3)c2c1]

### Example 5: 3-((1r,4r)-4-((3,5-dichloropyridin-2-yl)oxy)cyclohexyl)-2-oxo-2,3-dihydro-1H-benzo[d]imidazole-5-carboxylic acid

### (1r,4r)-4-((3,5-dichloropyridin-2-yl)oxy)cyclohexan-1-amine

To a solution of trans-4-aminocyclohexanol (520 mg, 4.52 mmol) in DMF (20 mL) was added 60% sodium hydride (542 mg, 13.6 mmol). The resulting mixture was stirred for 1 h, then 3,5-dichloro-2-fluoro-pyridine (899 mg, 5.42 mmol) was added. The contents were stirred for 4 h, then diluted with EtOAc. The organic layer was washed with 5% Na₂CO₃ (lx), water (3×), brine (lx), dried over MgSO₄, filtered and the solvent removed in vacuo to give a residue which was purified by automated normal-phase chromatography (0-100% MeOH/DCM, 40 g silica gel cartridge) to give (1r,4r)-4-((3,5-dichloropyridin-2-yl)oxy)cyclohexan-1-amine (316.9 mg, 1.214 mmol, 26.9% yield) as a brown syrup.

¹H NMR (400 MHz, DMSO-d₆) δ ppm 8.20 (d, J=2.27 Hz, 1H) 8.15 (d, J=2.53 Hz, 1H) 4.94 (tt, J=10.42, 4.11 Hz, 1H) 2.64 (tt, J=10.23, 3.66 Hz, 1H) 1.99-2.07 (m, 2H) 1.80 (dd, J=13.14, 2.78 Hz, 2H) 1.40-1.51 (m, 2H) 1.11-1.22 (m, 2H)

LCMS: [M+1]=261/263/265, rt=2.02 min.

### Methyl 3-(((1r,4r)-4-((3,5-dichloropyridin-2-yl)oxy)cyclohexyl)amino)-4-nitrobenzoate

A mixture of methyl 3-fluoro-4-nitro-benzoate (100 mg, 0.502 mmol), (1r,4r)-4-((3,5-dichloropyridin-2-yl)oxy)cyclohexan-1-amine (157 mg, 0.603 mmol) and cesium carbonate (196 mg, 0.603 mmol) in MeCN (5 mL) was stirred at 50° C. for 24 h, then at 30° C. for 3 days. The contents were taken up in EtOAc, washed with water (3×), brine (lx), dried over MgSO₄, filtered and the solvent removed in vacuo to give a residue which was purified by automated normal-phase chromatography (0-100% EtOAc/heptane, 20 g silica gel cartridge) to give methyl 3-(((1r,4r)-4-((3,5-dichloropyridin-2-yl)oxy)cyclohexyl)amino)-4-nitrobenzoate (151 mg, 0.343 mmol, 68.2% yield) as an orange solid.

¹H NMR (400 MHz, DMSO-d₆) δ ppm 8.22 (d, J=2.36 Hz, 1H) 8.19 (d, J=8.96 Hz, 1H) 8.17 (d, J=2.36 Hz, 1H) 7.93 (d, J=7.86 Hz, 1H) 7.61 (d, J=1.41 Hz, 1H) 7.16 (dd, J 8.88, 1.65 Hz, 1H) 5.05-5.12 (m, 1H) 3.83-3.91 (m, 4H) 2.05-2.16 (m, 4H) 1.68-1.78 (m, 2H) 1.57-1.67 (m, 2H)

LCMS: [M−1+23]=440/442/444, rt=2.87 min (lipophilic method).

### Methyl 4-amino-3-(((1r,4r)-4-((3,5-dichloropyridin-2-yl)oxy)cyclohexyl)amino)benzoate

A mixture of methyl 3-(((1r,4r)-4-((3,5-dichloropyridin-2-yl)oxy)cyclohexyl)amino)-4-nitrobenzoate (151 mg, 0.343 mmol), zinc powder (224 mg, 3.43 mmol) and ammonium chloride (183 mg, 3.43 mmol) in ethanol (3 mL) and water (3 mL) was stirred at 80° C. for 1 h. The contents were diluted with EtOAc, filtered through Celite and the solvent removed in vacuo to give methyl 4-amino-3-(((1r,4r)-4-((3,5-dichloropyridin-2-yl)oxy)cyclohexyl)amino)benzoate (149 mg, 0.362 mmol, 105% yield) as a yellow solid. This material was used without further purification.

¹H NMR (400 MHz, DMSO-d₆) δ ppm 8.21 (d, J=2.36 Hz, 1H) 8.16 (d, J=2.36 Hz, 1H) 7.13 (dd, J=8.10, 1.49 Hz, 1H) 7.04 (s, 1H) 6.55 (d, J=8.02 Hz, 1H) 5.45 (br. s., 2H) 5.00-5.08 (m, 1H) 4.40 (d, J=5.82 Hz, 1H) 3.73 (s, 4H) 3.34-3.39 (m, 1H) 2.11-2.18 (m, 2H) 2.07 (d, J=10.85 Hz, 2H) 1.57-1.67 (m, 2H) 1.33-1.43 (m, 2H)

LCMS: [M+1]=410/412/414, rt=1.48 min (lipophilic method).

### Methyl 3-((1r,4r)-4-((3,5-dichloropyridin-2-yl)oxy)cyclohexyl)-2-oxo-2,3-dihydro-1H-benzo[d]imidazole-5-carboxylate

A mixture of methyl 4-amino-3-(((1r,4r)-4-((3,5-dichloropyridin-2-yl)oxy)cyclohexyl)amino)benzoate (149 mg, 0.362 mmol) and 1,1′-carbonyldiimidazole (117 mg, 0.724 mmol) in chloroform (5 mL) was stirred at 60° C. for 18 h. The reaction mixture was reduced in volume with a stream of N₂ and purified by automated normal-phase chromatography (0-100% EtOAc/heptane, 20 g silica gel cartridge) to give methyl 3-((1r,4r)-4-((3,5-dichloropyridin-2-yl)oxy)cyclohexyl)-2-oxo-2,3-dihydro-1H-benzo[d]imidazole-5-carboxylate (94.4 mg, 0.216 mmol, 59.7% yield) as an off-white solid.

¹H NMR (400 MHz, DMSO-d₆) δ ppm 11.31 (s, 1H) 8.25 (d, J=2.36 Hz, 1H) 8.18 (d, J=2.36 Hz, 1H) 7.82 (s, 1H) 7.69 (dd, J=8.17, 1.41 Hz, 1H) 7.08 (d, J=8.17 Hz, 1H) 5.08-5.16 (m, 1H) 4.31-4.40 (m, 1H) 3.85 (s, 3H) 2.30-2.42 (m, 2H) 2.23 (d, J=10.06 Hz, 2H) 1.83 (d, J=11.63 Hz, 2H) 1.66-1.78 (m, 2H)

LCMS: [M+1]=436/438/440, rt=1.81 min (lipophilic method).

### 3-((1r,4r)-4-((3,5-dichloropyridin-2-yl)oxy)cyclohexyl)-2-oxo-2,3-dihydro-1H-benzo[d]imidazole-5-carboxylic acid

To a suspension of methyl 3-((1r,4r)-4-((3,5-dichloropyridin-2-yl)oxy)cyclohexyl)-2-oxo-2,3-dihydro-1H-benzo[d]imidazole-5-carboxylate (94.4 mg, 0.216 mmol) in methanol (8 mL) was added 1 N NaOH (1.08 mL, 1.08 mmol). the resulting mixture was stirred at 60° C. for 24 h. The solvent was removed under a stream of N₂, and the residue taken up in water and filtered through a syringe filter. The solution was acidified by addition of 6 N HCl and stirred for 3 h. The solid was collected by filtration, washed with water and dried under vacuum to give 3-((1r,4r)-4-((3,5-dichloropyridin-2-yl)oxy)cyclohexyl)-2-oxo-2,3-dihydro-1H-benzo[d]imidazole-5-carboxylic acid (64.4 mg, 0.153 mmol, 70.5% yield) as a tan solid.

¹H NMR (400 MHz, DMSO-d₆) δ ppm 12.62 (br. s, 1H) 11.27 (s, 1H) 8.25 (d, J 2.36 Hz, 1H) 8.17 (d, J=2.36 Hz, 1H) 7.80 (s, 1H) 7.67 (dd, J=8.17, 1.41 Hz, 1H) 7.06 (d, J=8.17 Hz, 1H) 5.08-5.17 (m, 1H) 4.30-4.40 (m, 1H) 2.29-2.41 (m, 2H) 2.23 (d, J=11.16 Hz, 2H) 1.84 (d, J=11.48 Hz, 2H) 1.66-1.77 (m, 2H)

LCMS: [M+1]=422/424/426, rt=1.35 min (lipophilic method).

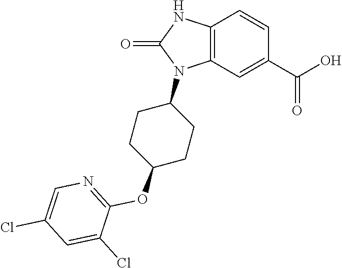 [SMILES: O=C(O)c1ccc2[nH]c(=O)n([C@H]3CC[C@@H](Oc4ncc(Cl)cc4Cl)CC3)c2c1]

### Example 6: 3-((1s,4s)-4-((3,5-dichloropyridin-2-yl)oxy)cyclohexyl)-2-oxo-2,3-dihydro-1H-benzo[d]imidazole-5-carboxylic acid

### (1s,4s)-4-((3,5-dichloropyridin-2-yl)oxy)cyclohexan-1-amine

To a solution of cis-4-aminocyclohexanol hydrochloride (250 mg, 1.65 mmol) in DMF (15 mL) was added 60% sodium hydride (264 mg, 6.60 mmol) portionwise over 10 min. The resulting mixture was stirred for 15 min, then 3,5-dichloro-2-fluoro-pyridine (328 mg, 1.98 mmol) was added. The contents were stirred for 16 h, then diluted with EtOAc. The organic layer was washed with 5% Na₂CO₃ (lx), water (3×), brine (lx), dried over MgSO₄, filtered and the solvent removed in vacuo to give a residue which was purified by automated normal-phase chromatography (0-100% MeOH/DCM, 20 g silica gel cartridge) to give (1s,4s)-4-((3,5-dichloropyridin-2-yl)oxy)cyclohexan-1-amine (168.7 mg, 0.646 mmol, 39.2% yield) as a beige solid.

¹H NMR (400 MHz, DMSO-d₆) δ ppm 8.18 (d, J=2.36 Hz, 1H) 8.14 (d, J=2.36 Hz, 1H) 5.15 (d, J=2.67 Hz, 1H) 2.69-2.76 (m, 1H) 1.91 (dt, J=8.84, 4.46 Hz, 2H) 1.54-1.66 (m, 4H) 1.37-1.46 (m, 2H)

LCMS: [M+1]=261/263/265, rt=2.02 min.

### Methyl 3-(((1s,4s)-4-((3,5-dichloropyridin-2-yl)oxy)cyclohexyl)amino)-4-nitrobenzoate

A mixture of methyl 3-fluoro-4-nitro-benzoate (105 mg, 0.527 mmol), (1s,4s)-4-((3,5-dichloropyridin-2-yl)oxy)cyclohexan-1-amine (165 mg, 0.633 mmol) and cesium carbonate (206 mg, 0.633 mmol) in MeCN (5 mL) was stirred at 60° C. for 20 h. The contents were taken up in EtOAc, washed with water (3×), brine (lx), dried over MgSO₄, filtered and the solvent removed in vacuo to give a residue which was purified by automated normal-phase chromatography (0-50% EtOAc/heptane, 20 g silica gel cartridge) to give methyl 3-(((1s,4s)-4-((3,5-dichloropyridin-2-yl)oxy)cyclohexyl)amino)-4-nitrobenzoate (196.2 mg, 0.446 mmol, 84.5% yield) as an orange foam.

¹H NMR (400 MHz, DMSO-d₆) δ ppm 8.17-8.22 (m, 3H) 8.01 (d, J=7.70 Hz, 1H) 7.60 (d, J=1.41 Hz, 1H) 7.17 (dd, J=8.88, 1.65 Hz, 1H) 5.27 (br. s., 1H) 3.87-3.94 (m, 4H) 1.93 (br. s., 6H) 1.71-1.81 (m, 2H)

LCMS: [M−1+23]=440/442/444, rt=2.84 min (lipophilic method).

### Methyl 4-amino-3-(((1s,4s)-4-((3,5-dichloropyridin-2-yl)oxy)cyclohexyl)amino)benzoate

A mixture of methyl 3-(((1s,4s)-4-((3,5-dichloropyridin-2-yl)oxy)cyclohexyl)amino)-4-nitrobenzoate (196 mg, 0.445 mmol), zinc powder (291 mg, 4.45 mmol) and ammonium chloride (238 mg, 4.45 mmol) in ethanol (3 mL) and water (3 mL) was stirred at 80° C. for 2 h. The contents were diluted with EtOAc, filtered through Celite and the solvent removed in vacuo to give methyl 4-amino-3-(((1s,4s)-4-((3,5-dichloropyridin-2-yl)oxy)cyclohexyl)amino)benzoate (177 mg, 0.431 mmol, 96.8% yield) as a pink foam.

¹H NMR (400 MHz, DMSO-d₆) δ ppm 8.21 (d, J=2.36 Hz, 1H) 8.16 (d, J=2.36 Hz, 1H) 7.12 (dd, J=8.17, 1.73 Hz, 1H) 7.03 (s, 1H) 6.54 (d, J=8.17 Hz, 1H) 5.48-5.53 (m, 2H) 5.25 (br. s., 1H) 4.45 (d, J=7.07 Hz, 1H) 3.72 (s, 3H) 3.39 (d, J 7.07 Hz, 1H) 1.97-2.04 (m, 2H) 1.76-1.88 (m, 4H) 1.62-1.71 (m, 2H)

LCMS: [M+1]=410/412/414, rt=1.43 min (lipophilic method).

### Methyl 3-((1s,4s)-4-((3,5-dichloropyridin-2-yl)oxy)cyclohexyl)-2-oxo-2,3-dihydro-1H-benzo[d]imidazole-5-carboxylate

A mixture of methyl 4-amino-3-(((1s,4s)-4-((3,5-dichloropyridin-2-yl)oxy)cyclohexyl)amino)benzoate (176 mg, 0.429 mmol) and 1,1′-carbonyldiimidazole (139 mg, 0.858 mmol) in chloroform (8 mL) was stirred at 60° C. for 4 h. The solvent was removed under a stream of N₂, and the residue triturated with MeOH for 1 h and cooled to 0° C. The solid was collected by filtration, washed with cold MeOH and dried to give methyl 3-((1s,4s)-4-((3,5-dichloropyridin-2-yl)oxy)cyclohexyl)-2-oxo-2,3-dihydro-1H-benzo[d]imidazole-5-carboxylate (144.3 mg, 0.331 mmol, 77.1% yield) as an off-white solid.

¹H NMR (400 MHz, DMSO-d₆) δ ppm 11.35 (s, 1H) 8.23 (s, 2H) 7.88 (s, 1H) 7.70 (dd, J=8.17, 1.26 Hz, 1H) 7.10 (d, J=8.17 Hz, 1H) 5.42 (br. s., 1H) 4.38-4.46 (m, 1H) 3.84 (s, 3H) 2.40-2.48 (m, 2H) 2.10 (d, J=14.15 Hz, 2H) 1.80-1.90 (m, 2H) 1.63 (d, J=12.10 Hz, 2H)

LCMS: [M+1]=436/438/440, rt=1.79 min (lipophilic method).

### 3-((1s,4s)-4-((3,5-dichloropyridin-2-yl)oxy)cyclohexyl)-2-oxo-2,3-dihydro-1H-benzo[d]imidazole-5-carboxylic acid

To a suspension of methyl 3-((1s,4s)-4-((3,5-dichloropyridin-2-yl)oxy)cyclohexyl)-2-oxo-2,3-dihydro-1H-benzo[d]imidazole-5-carboxylate (144.3 mg, 0.331 mmol) in methanol (5 mL) was added 1 N NaOH (1.65 mL, 1.65 mmol). The resulting mixture was stirred at 60° C. for 6 h, at 40° C. for 65 h, then at 60° C. for 26 h. The solvent was removed under a stream of N₂, and the residue taken up in water and filtered through a syringe filter. The solution was acidified by addition of 6 N HCl and stirred for 18 h.

The solid was collected by filtration, washed with water and dried under vacuum to give 3-((1s,4s)-4-((3,5-dichloropyridin-2-yl)oxy)cyclohexyl)-2-oxo-2,3-dihydro-1H-benzo[d]imidazole-5-carboxylic acid (119.8 mg, 0.284 mmol, 85.8% yield) as a beige solid.

¹H NMR (400 MHz, DMSO-d₆) δ ppm 12.72 (br. s., 1H) 11.31 (s, 1H) 8.21-8.24 (m, 1H) 8.19 (d, J=2.27 Hz, 1H) 7.92 (s, 1H) 7.67 (dd, J=8.21, 1.39 Hz, 1H) 7.07 (d, J=8.08 Hz, 1H) 5.44 (br. s., 1H) 4.35-4.47 (m, 1H) 2.42-2.54 (m, 2H) 2.08 (d, J=12.88 Hz, 2H) 1.78-1.92 (m, 2H) 1.63 (d, J=11.87 Hz, 2H)

LCMS: [M+1]=422/424/426, rt=1.22 min (lipophilic method).

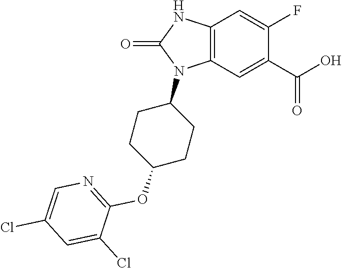 [SMILES: O=C(O)c1cc2c(cc1F)[nH]c(=O)n2[C@H]1CC[C@H](Oc2ncc(Cl)cc2Cl)CC1]

### Example 7: 3-((1r,4r)-4-((3,5-dichloropyridin-2-yl)oxy)cyclohexyl)-6-fluoro-2-oxo-2,3-dihydro-1H-benzo[d]imidazole-5-carboxylic acid

### Methyl 3-(((1r,4r)-4-((3,5-dichloropyridin-2-yl)oxy)cyclohexyl)amino)-2-fluoro-4-nitrobenzoate

A mixture of methyl 2,5-difluoro-4-nitrobenzoate (110 mg, 0.507 mmol), (1r,4r)-4-((3,5-dichloropyridin-2-yl)oxy)cyclohexan-1-amine (159 mg, 0.608 mmol) and cesium carbonate (198 mg, 0.608 mmol) in MeCN (5 mL) was stirred at 30° C. for 8 days. The contents were taken up in EtOAc, washed with water (3×), brine (lx), dried over MgSO₄, filtered and the solvent removed in vacuo to give a residue which was purified by automated normal-phase chromatography (0-100% EtOAc/heptane, 20 g silica gel cartridge) to give methyl 3-(((1r,4r)-4-((3,5-dichloropyridin-2-yl)oxy)cyclohexyl)amino)-2-fluoro-4-nitrobenzoate (80.0 mg, 0.175 mmol, 34.5% yield) as an orange solid.

¹H NMR (400 MHz, DMSO-d₆) δ ppm 8.23 (d, J=2.27 Hz, 1H) 8.19 (d, J=2.27 Hz, 1H) 8.02 (d, J=10.86 Hz, 1H) 7.79 (d, J=8.08 Hz, 1H) 7.52 (d, J=5.81 Hz, 1H) 5.09 (d, J=9.09 Hz, 1H) 3.90 (s, 3H) 3.82 (br. s., 1H) 2.02-2.16 (m, 4H) 1.54-1.77 (m, 4H)

¹⁹F NMR (376 MHz, DMSO-d₆) δ ppm −229.08 (dd, J=10.33, 5.74 Hz, 1F)

LCMS: [M−1+23]=458/460/462, rt=2.67 min (lipophilic method).

### Methyl 4-amino-3-(((1r,4r)-4-((3,5-dichloropyridin-2-yl)oxy)cyclohexyl)amino)-2-fluorobenzoate

A mixture of methyl 3-(((1r,4r)-4-((3,5-dichloropyridin-2-yl)oxy)cyclohexyl)amino)-2-fluoro-4-nitrobenzoate (80.0 mg, 0.175 mmol), zinc powder (114 mg, 1.75 mmol) and ammonium chloride (93.4 mg, 1.75 mmol) in ethanol (3 mL) and water (3 mL) was stirred at 80° C. for 2 h. The contents were diluted with EtOAc, filtered through Celite and the solvent removed in vacuo to give methyl 4-amino-3-(((1r,4r)-4-((3,5-dichloropyridin-2-yl)oxy)cyclohexyl)amino)-2-fluorobenzoate (89.0 mg, 0.208 mmol, 119% yield) as a dark red gum and was used without further purification.

¹H NMR (400 MHz, DMSO-d₆) δ ppm 8.22 (d, J=2.27 Hz, 1H) 8.17 (d, J=2.53 Hz, 1H) 7.21 (br. s., 1H) 6.87 (d, J=7.07 Hz, 1H) 6.33 (d, J=13.39 Hz, 1H) 5.86 (s, 2H) 4.98-5.08 (m, 1H) 4.32 (d, J=7.07 Hz, 1H) 3.73 (s, 3H) 3.20-3.30 (m, 1H) 2.13 (d, J=9.09 Hz, 2H) 2.05 (d, J=10.36 Hz, 2H) 1.53-1.66 (m, 2H) 1.29-1.42 (m, 2H)

¹⁹F NMR (376 MHz, DMSO-d₆) δ ppm −220.67 (dd, J=13.20, 7.46 Hz, 1F)

LCMS: [M+1]=428/430/432, rt=1.46 min (lipophilic method).

### Methyl 3-((1r,4r)-4-((3,5-dichloropyridin-2-yl)oxy)cyclohexyl)-6-fluoro-2-oxo-2,3-dihydro-1H-benzo[d]imidazole-5-carboxylate

A mixture of methyl 4-amino-3-(((1r,4r)-4-((3,5-dichloropyridin-2-yl)oxy)cyclohexyl)amino)-2-fluorobenzoate (89.0 mg, 0.208 mmol) and 1,1′-carbonyldiimidazole (33.7 mg, 0.208 mmol) in chloroform (6 mL) was stirred at 60° C. for 17 h. The solvent was removed with a stream of N₂ and the residue purified by automated normal-phase chromatography (0-100% EtOAc/heptane, 12 g silica gel cartridge) to give methyl 3-((1r,4r)-4-((3,5-dichloropyridin-2-yl)oxy)cyclohexyl)-6-fluoro-2-oxo-2,3-dihydro-1H-benzo[d]imidazole-5-carboxylate (38.7 mg, 0.0852 mmol, 41.0% yield) as a pale orange solid.

¹H NMR (400 MHz, DMSO-d₆) δ ppm 11.47 (br. s., 1H) 8.25 (d, J=2.27 Hz, 1H) 8.19 (d, J=2.27 Hz, 1H) 7.70 (d, J=5.81 Hz, 1H) 6.94 (d, J=10.86 Hz, 1H) 5.07-5.17 (m, 1H) 4.34 (t, J=12.63 Hz, 1H) 3.85 (s, 3H) 2.30-2.37 (m, 2H) 2.22 (d, J=10.86 Hz, 2H) 1.82 (d, J=10.86 Hz, 2H) 1.70 (q, J=11.45 Hz, 2H)

¹⁹F NMR (376 MHz, DMSO-d₆) δ ppm −217.01 (br. s., 1F)

LCMS: [M+1]=454/456/458, rt=1.71 min (lipophilic method).

### 3-((1r,4r)-4-((3,5-dichloropyridin-2-yl)oxy)cyclohexyl)-6-fluoro-2-oxo-2,3-dihydro-1H-benzo[d]imidazole-5-carboxylic acid

A mixture of methyl 3-((1r,4r)-4-((3,5-dichloropyridin-2-yl)oxy)cyclohexyl)-6-fluoro-2-oxo-2,3-dihydro-1H-benzo[d]imidazole-5-carboxylate (38.7 mg, 0.0852 mmol) and lithium hydroxide monohydrate (3.6 mg, 0.0852 mmol) in THF (4 mL) and water (1 mL) was stirred at 60° C. for 46 h. The solvent was removed with a stream of N₂, and the residue taken up in water. The solution was acidified by addition of 6 N HCl and stirred for 3 days (for convenience). The solid was collected by filtration, washed with water and dried under vacuum to give 3-((1r,4r)-4-((3,5-dichloropyridin-2-yl)oxy)cyclohexyl)-6-fluoro-2-oxo-2,3-dihydro-1H-benzo[d]imidazole-5-carboxylic acid (35.6 mg, 0.0809 mmol, 94.9% yield) as a tan solid.

¹H NMR (400 MHz, DMSO-d₆) δ ppm 12.99 (br. s., 1H) 11.41 (s, 1H) 8.26 (d, J 2.53 Hz, 1H) 8.19 (d, J=2.53 Hz, 1H) 7.68 (d, J=5.81 Hz, 1H) 6.90 (d, J=10.86 Hz, 1H) 5.06-5.17 (m, 1H) 4.33 (br. s., 1H) 2.32 (d, J=13.39 Hz, 2H) 2.22 (d, J=10.86 Hz, 2H) 1.82 (d, J=13.39 Hz, 2H) 1.63-1.76 (m, 2H)

¹⁹F NMR (376 MHz, DMSO-d₆) δ ppm −216.89 (br. s., 1F)

LCMS: [M+23]=462/464/466, parent 277, rt=1.31 min (lipophilic method).

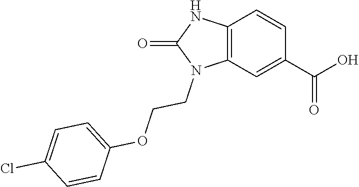 [SMILES: O=C(O)c1ccc2[nH]c(=O)n(CCOc3ccc(Cl)cc3)c2c1]

### Example 8: 3-(2-(4-chlorophenoxy)ethyl)-2-oxo-2,3-dihydro-1H-benzo[d]imidazole-5-carboxylic acid

### Methyl 3-((2-(4-chlorophenoxy)ethyl)amino)-4-nitrobenzoate

A mixture of methyl 3-fluoro-4-nitrobenzoate (100 mg, 0.502 mmol), 2-(4-chlorophenoxy)ethan-1-amine (103 mg, 0.603 mmol) and cesium carbonate (229 mg, 0.703 mmol) in MeCN (10 mL) was stirred at 60° C. for 18 h. The contents were taken up in EtOAc, washed with water (3×), brine (lx), dried over MgSO₄, filtered and the solvent removed in vacuo to give a residue which was purified by automated normal-phase chromatography (0-50% EtOAc/heptane, 12 g silica gel cartridge) to give methyl 3-((2-(4-chlorophenoxy)ethyl)amino)-4-nitrobenzoate (150 mg, 0.428 mmol, 85.2% yield) as an orange solid.

¹H NMR (400 MHz, DMSO-d₆) δ ppm 8.33 (t, J=5.74 Hz, 1H) 8.18 (d, J=8.80 Hz, 1H) 7.71 (d, J=1.57 Hz, 1H) 7.30-7.36 (m, 2H) 7.18 (dd, J=8.88, 1.65 Hz, 1H) 6.96-7.01 (m, 2H) 4.26 (t, J=5.27 Hz, 2H) 3.89 (s, 3H) 3.81 (q, J=5.50 Hz, 2H)

LCMS: [M−1+23]=351/353, rt=1.97 min (lipophilic method).

### Methyl 4-amino-3-((2-(4-chlorophenoxy)ethyl)amino)benzoate

To a solution of methyl 3-((2-(4-chlorophenoxy)ethyl)amino)-4-nitrobenzoate (150 mg, 0.428 mmol) in ethyl acetate (20 mL) was added 10% Pd/C (20. mg, 0.4300 mmol). the resulting suspension was stirred under a H₂ atmosphere (balloon) for 4 h. An 85:15 mix of desired product and des-chloro byproduct was seen by LCMS. The contents were filtered through Celite and the solvent removed in vacuo to give a pale yellow solid which was purified by automated normal-phase chromatography (0-100% EtOAc/heptane, 12 g silica gel cartridge) to give methyl 4-amino-3-((2-(4-chlorophenoxy)ethyl)amino)benzoate (49.2 mg, 0.153 mmol, 35.9% yield) as a beige solid.

¹H NMR (400 MHz, DMSO-d₆) δ ppm 7.31-7.36 (m, 2H) 7.18 (dd, J=8.10, 1.81 Hz, 1H) 7.06 (d, J=1.57 Hz, 1H) 6.99-7.03 (m, 2H) 6.56 (d, J=8.17 Hz, 1H) 5.47 (s, 2H) 4.84 (t, J=5.42 Hz, 1H) 4.18 (t, J=5.42 Hz, 2H) 3.73 (s, 3H) 3.45 (q, J=5.34 Hz, 2H)

LCMS: [M+1]=321, rt=1.33 min (lipophilic method).

### Methyl 3-(2-(4-chlorophenoxy)ethyl)-2-oxo-2,3-dihydro-1H-benzo[d]imidazole-5-carboxylate

A mixture of methyl 4-amino-3-((2-(4-chlorophenoxy)ethyl)amino)benzoate (49.2 mg, 0.153 mmol) and 1,1′-carbonyldiimidazole (49.7 mg, 0.307 mmol) in chloroform (5 mL) was stirred at 60° C. for 4 h. The solvent was removed under a stream of N₂, and the residue triturated with MeOH for 18 h. The solid was collected by filtration, washed with cold MeOH and dried to give methyl 3-(2-(4-chlorophenoxy)ethyl)-2-oxo-2,3-dihydro-1H-benzo[d]imidazole-5-carboxylate (30.0 mg, 0.0865 mmol, 56.4% yield) as a white solid.

¹H NMR (400 MHz, DMSO-d₆) δ ppm 11.37 (s, 1H) 7.83 (d, J=1.26 Hz, 1H) 7.67-7.71 (m, 1H) 7.27-7.33 (m, 2H) 7.08 (d, J=8.34 Hz, 1H) 6.86-6.92 (m, 2H) 4.24 (s, 4H) 3.85 (s, 3H)

LCMS: [M+1]=347/349, rt=1.29 min (lipophilic method).

### 3-(2-(4-chlorophenoxy)ethyl)-2-oxo-2,3-dihydro-1H-benzo[d]imidazole-5-carboxylic acid

To a suspension of methyl 3-(2-(4-chlorophenoxy)ethyl)-2-oxo-2,3-dihydro-1H-benzo[d]imidazole-5-carboxylate (30.0 mg, 0.0865 mmol) in methanol (5 mL) was added 1 N NaOH (433 μL, 0.433 mmol). The resulting mixture was stirred at 60° C. for 1 h, at 40° C. for 65 h, then at 60° C. for 24 h. The solvent was removed under a stream of N₂, and the residue taken up in water and filtered through a syringe filter. The solution was acidified by addition of 6 N HCl and stirred for 18 h. The solid was collected by filtration, washed with water and dried under vacuum to give 3-(2-(4-chlorophenoxy)ethyl)-2-oxo-2,3-dihydro-1H-benzo[d]imidazole-5-carboxylic acid (24.4 mg, 0.0733 mmol, 84.8% yield) as an off-white solid.

¹H NMR (400 MHz, DMSO-d₆) δ ppm 12.71 (br. s., 1H) 11.31 (s, 1H) 7.84 (d, J 1.26 Hz, 1H) 7.67 (dd, J=8.34, 1.52 Hz, 1H) 7.26-7.32 (m, 2H) 7.05 (d, J=8.34 Hz, 1H) 6.85-6.92 (m, 2H) 4.24 (s, 4H)

LCMS: [M+1]=333, rt=1.03 min (lipophilic method).

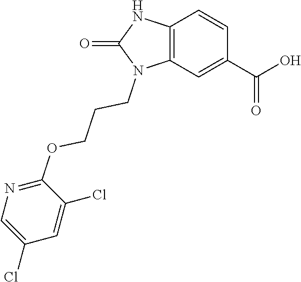 [SMILES: O=C(O)c1ccc2[nH]c(=O)n(CCCOc3ncc(Cl)cc3Cl)c2c1]

### Example 9: 3-(3-((3,5-dichloropyridin-2-yl)oxy)propyl)-2-oxo-2,3-dihydro-1H-benzo[d]imidazole-5-carboxylic acid

### Methyl 3-((3-((3,5-dichloropyridin-2-yl)oxy)propyl)amino)-4-nitrobenzoate

A mixture of methyl 3-fluoro-4-nitrobenzoate (177 mg, 0.889 mmol), 3-((3,5-dichloropyridin-2-yl)oxy)propan-1-amine (236 mg, 1.07 mmol) and Cesium carbonate (348 mg, 1.07 mmol) in MeCN (15 mL) was stirred at 70° C. for 3 h. The contents were taken up in EtOAc, washed with water (3×), brine (lx), dried over MgSO₄, filtered and the solvent removed in vacuo to give a residue which was purified by automated normal-phase chromatography (0-25% EtOAc/heptane, 20 g silica gel cartridge) to give methyl 3-((3-((3,5-dichloropyridin-2-yl)oxy)propyl)amino)-4-nitrobenzoate (270 mg, 0.675 mmol, 75.9% yield) as a yellow-orange solid.

¹H NMR (400 MHz, DMSO-d₆) δ ppm 8.27 (t, J=5.68 Hz, 1H) 8.15-8.19 (m, 3H) 7.53 (d, J=1.77 Hz, 1H) 7.13 (dd, J=8.84, 1.77 Hz, 1H) 4.44 (t, J=6.06 Hz, 2H) 3.86 (s, 3H) 3.59 (q, J=6.48 Hz, 2H) 2.13 (t, J=6.32 Hz, 2H)

LCMS: [M+1]=400/402/404, rt=2.16 min (lipophilic method).

### Methyl 4-amino-3-((3-((3,5-dichloropyridin-2-yl)oxy)propyl)amino)benzoate

A mixture of methyl 3-((3-((3,5-dichloropyridin-2-yl)oxy)propyl)amino)-4-nitrobenzoate (270 mg, 0.675 mmol), zinc powder (441 mg, 6.75 mmol) and ammonium chloride (361 mg, 6.75 mmol) in ethanol (3 mL) and water (3 mL) was stirred at 80° C. for 3 h. The contents were diluted with EtOAc, filtered through Celite and the solvent removed in vacuo to give methyl 4-amino-3-((3-((3,5-dichloropyridin-2-yl)oxy)propyl)amino)benzoate (309 mg, 0.835 mmol, 123% yield) as a tan solid. This material was carried forward without further purification.

¹H NMR (400 MHz, DMSO-d₆) δ ppm 8.22 (d, J=2.53 Hz, 1H) 8.19 (d, J=2.27 Hz, 1H) 7.16 (dd, J=8.08, 1.77 Hz, 1H) 6.98 (d, J=1.77 Hz, 1H) 6.54 (d, J=8.08 Hz, 1H) 5.46 (s, 2H) 4.70 (t, J=5.43 Hz, 1H) 4.47 (t, J=6.32 Hz, 2H) 3.18-3.26 (m, 2H) 2.09 (t, J=6.44 Hz, 2H)

LCMS: [M+1]=370/372/374, rt=1.31 min (lipophilic method).

### Methyl 3-(3-((3,5-dichloropyridin-2-yl)oxy)propyl)-2-oxo-2,3-dihydro-1H-benzo[d]imidazole-5-carboxylate

A mixture of methyl 4-amino-3-((3-((3,5-dichloropyridin-2-yl)oxy)propyl)amino)benzoate (250 mg, 0.675 mmol) and 1,1′-carbonyldiimidazole (219 mg, 1.35 mmol) in chloroform (8 mL) was stirred at 60° C. for 22 h. The solvent was removed with a stream of N₂ and the residue triturated with water/MeOH (1:1) for 18 h. The solvent was collected by filtration, washed with water and dried under vacuum to give methyl 3-(3-((3,5-dichloropyridin-2-yl)oxy)propyl)-2-oxo-2,3-dihydro-1H-benzo[d]imidazole-5-carboxylate (263 mg, 0.665 mmol, 98.4% yield) as an off-white solid.

¹H NMR (400 MHz, DMSO-d₆) δ ppm 11.32 (s, 1H) 8.17 (d, J=2.02 Hz, 1H) 8.13 (d, J=2.27 Hz, 1H) 7.65 (dd, J=8.21, 1.64 Hz, 1H) 7.60 (d, J=1.52 Hz, 1H) 7.07 (d, J=8.34 Hz, 1H) 4.32 (t, J=5.81 Hz, 2H) 4.02 (t, J=6.44 Hz, 2H) 3.79 (s, 3H) 2.14 (quin, J=6.13 Hz, 2H)

LCMS: [M+1]=396/398/400, rt=1.32 min (lipophilic method).

### 3-(3-((3,5-dichloropyridin-2-yl)oxy)propyl)-2-oxo-2,3-dihydro-1H-benzo[d]imidazole-5-carboxylic acid

To a suspension of methyl 3-(3-((3,5-dichloropyridin-2-yl)oxy)propyl)-2-oxo-2,3-dihydro-1H-benzo[d]imidazole-5-carboxylate (263 mg, 0.665 mmol) in methanol (10 mL) was added 1 N NaOH (6.65 mL, 6.65 mmol). The resulting mixture was stirred at 60° C. for 18 h. After cooling, the solvent volume was reduced with a stream of N₂ and the contents acidified by addition of 6 N HCl. The precipitate was collected by filtration, washed with water and dried under vacuum to give 3-(3-((3,5-dichloropyridin-2-yl)oxy)propyl)-2-oxo-2,3-dihydro-1H-benzo[d]imidazole-5-carboxylic acid (217 mg, 0.568 mmol, 85.5% yield) as an off-white solid.

¹H NMR (400 MHz, DMSO-d₆) δ ppm 12.64 (br. s., 1H) 11.25 (s, 1H) 8.14-8.15 (m, 1H) 8.13-8.14 (m, 1H) 7.64 (td, J=4.29, 1.52 Hz, 2H) 7.04 (d, J=8.34 Hz, 1H) 4.34 (t, J=5.94 Hz, 2H) 4.01 (t, J=6.69 Hz, 2H) 2.13 (t, J=6.19 Hz, 2H)

LCMS: [M+1]=382/384/386, rt=1.07 min (lipophilic method).

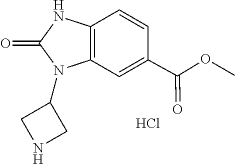 [SMILES: COC(=O)c1ccc2[nH]c(=O)n(C3CNC3)c2c1 ; Cl]

### Intermediate B: Methyl 3-(azetidin-3-yl)-2-oxo-2,3-dihydro-1H-benzo[d]imidazole-5-carboxylate hydrochloride

### tert-butyl 3-((5-(methoxycarbonyl)-2-nitrophenyl)amino)azetidine-1-carboxylate

A mixture of methyl 3-fluoro-4-nitro-benzoate (307 mg, 1.54 mmol), tert-butyl 3-aminoazetidine-1-carboxylate (319 mg, 1.85 mmol) and cesium carbonate (603 mg, 1.85 mmol) in MeCN (5 mL) was stirred at 30° C. for 4 days, then at 60° C. for 6 h. The contents were taken up in EtOAc, washed with water (3×), brine (1×), dried over MgSO₄, filtered and the solvent removed in vacuo to give a residue which was purified by automated normal-phase chromatography (0-50% EtOAc/heptane, 20 g silica gel cartridge) to give tert-butyl 3-((5-(methoxycarbonyl)-2-nitrophenyl)amino)azetidine-1-carboxylate (340 mg, 0.968 mmol, 62.8% yield) as a yellow-orange foam.

¹H NMR (400 MHz, DMSO-d₆) δ ppm 8.20 (d, J=8.80 Hz, 1H) 8.13 (d, J=5.66 Hz, 1H) 7.26 (dd, J=8.72, 1.65 Hz, 1H) 7.23 (d, J=1.57 Hz, 1H) 4.48-4.56 (m, 1H) 4.24 (br. s., 2H) 3.85-3.91 (m, 5H) 1.39 (s, 9H)

LCMS: [M+23]=374, rt=1.62 min (lipophilic method).

### tert-butyl 3-((2-amino-5-(methoxycarbonyl)phenyl)amino)azetidine-1-carboxylate

To a solution of tert-butyl 3-((5-(methoxycarbonyl)-2-nitrophenyl)amino)azetidine-1-carboxylate (340 mg, 0.968 mmol) in ethyl acetate (25 mL) was added 10% Pd/C (34 mg, 0.097 mmol). The resulting suspension was stirred under a H₂ atmosphere (balloon) for 4 h. The contents were filtered through Celite and the solvent removed in vacuo to give tert-butyl 3-((2-amino-5-(methoxycarbonyl)phenyl)amino)azetidine-1-carboxylate (304 mg, 0.947 mmol, 97.9% yield) as a tan solid.

¹H NMR (400 MHz, DMSO-d₆) δ ppm 7.21 (dd, J=8.10, 1.81 Hz, 1H) 6.72 (d, J=1.73 Hz, 1H) 6.57 (d, J=8.17 Hz, 1H) 5.48 (s, 2H) 5.26 (d, J=6.13 Hz, 1H) 4.11-4.23 (m, 3H) 3.72 (s, 3H) 3.67 (dd, J=8.25, 4.17 Hz, 2H) 1.39 (s, 9H)

LCMS: [M+23]=344, rt=1.15 min (lipophilic method).

### Methyl 3-(1-(tert-butoxycarbonyl)azetidin-3-yl)-2-oxo-2,3-dihydro-1H-benzo[d]imidazole-5-carboxylate

A mixture of tert-butyl 3-((2-amino-5-(methoxycarbonyl)phenyl)amino)azetidine-1-carboxylate (304 mg, 0.947 mmol) and 1,1′-carbonyldiimidazole (276 mg, 1.70 mmol) in chloroform (8 mL) was stirred at 60° C. for 18 h. The solvent was removed under a stream of N₂, and the residue triturated with 12 mL MeOH for 1 h, then cooled to 0° C. The solid was collected by filtration, washed with cold MeOH and dried to give methyl 3-(1-(tert-butoxycarbonyl)azetidin-3-yl)-2-oxo-2,3-dihydro-1H-benzo[d]imidazole-5-carboxylate (284 mg, 0.819 mmol, 86.4% yield) as a white solid.

¹H NMR (400 MHz, DMSO-d₆) δ ppm 11.44 (br. s., 1H) 7.80 (d, J=1.10 Hz, 1H) 7.73 (dd, J=8.17, 1.41 Hz, 1H) 7.12 (d, J=8.17 Hz, 1H) 5.24 (tt, J=8.51, 5.56 Hz, 1H) 4.22-4.40 (m, 4H) 3.82 (s, 3H) 1.44 (s, 9H)

LCMS: [M+23]=370, rt=2.28 min.

### Methyl 3-(azetidin-3-yl)-2-oxo-2,3-dihydro-1H-benzo[d]imidazole-5-carboxylate hydrochloride

To a suspension of methyl 3-(1-(tert-butoxycarbonyl)azetidin-3-yl)-2-oxo-2,3-dihydro-1H-benzo[d]imidazole-5-carboxylate (284 mg, 0.819 mmol) in methanol (10 mL) was added 6 N HCl (1.36 mL, 8.19 mmol). The resulting mixture was stirred at 60° C. for 6 h. The contents were allowed to cool to room temperature and stirred over the weekend, then cooled to 0° C. The solid was collected by filtration, washed with cold MeOH and dried under vacuum to give methyl 3-(azetidin-3-yl)-2-oxo-2,3-dihydro-1H-benzo[d]imidazole-5-carboxylate hydrochloride (187 mg, 0.658 mmol, 80.3% yield) as a white solid.

¹H NMR (400 MHz, DMSO-d₆) δ ppm 11.66 (br. s., 1H) 9.18 (br. s., 2H) 7.82 (d, J 1.52 Hz, 1H) 7.73 (dd, J=8.34, 1.52 Hz, 1H) 7.13 (d, J=8.08 Hz, 1H) 5.38-5.48 (m, 1H) 4.68 (dd, J=11.50, 7.45 Hz, 2H) 4.27 (dd, J=11.37, 9.09 Hz, 2H) 3.85 (s, 3H)

LCMS: [M+1]=248, rt=1.24 min.

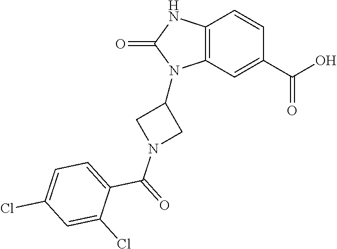 [SMILES: O=C(O)c1ccc2[nH]c(=O)n(C3CN(C(=O)c4ccc(Cl)cc4Cl)C3)c2c1]

### Example 10: 3-(1-(2,4-dichlorobenzoyl)azetidin-3-yl)-2-oxo-2,3-dihydro-1H-benzo[d]imidazole-5-carboxylic acid

### Methyl 3-(1-(2,4-dichlorobenzoyl)azetidin-3-yl)-2-oxo-2,3-dihydro-1H-benzo[d]imidazole-5-carboxylate

A mixture of methyl 3-(azetidin-3-yl)-2-oxo-2,3-dihydro-1H-benzo[d]imidazole-5-carboxylate hydrochloride (42.0 mg, 0.148 mmol), 2,4-dichlorobenzoic acid (29.7 mg, 0.155 mmol), HBTU (58.9 mg, 0.155 mmol) and DIPEA (77.4 μL, 0.444 mmol) in chloroform (5 mL) was stirred at room temperature for 18 h. The contents were treated with water and extracted with CHCl₃ (3×). The organic layers were filtered through a cotton plug, reduced in volume with a stream of N₂ and then purified by automated normal-phase chromatography (0-20% MeOH/DCM, 4 g silica gel cartridge) to give methyl 3-(1-(2,4-dichlorobenzoyl)azetidin-3-yl)-2-oxo-2,3-dihydro-1H-benzo[d]imidazole-5-carboxylate (30.4 mg, 0.0723 mmol, 48.9% yield) as a white solid. Two amide conformers by 1H NMR—one peak by LCMS.

¹H NMR (400 MHz, DMSO-d₆) δ ppm 11.50 (s, 1H) 7.12-7.92 (m, 6H) 5.33-5.43 (m, 1H) 4.25-4.54 (m, 4H) 3.85 (s, 3H)

LCMS: [M+1]=420/422/424, rt=2.30 min.

### 3-(1-(2,4-dichlorobenzoyl)azetidin-3-yl)-2-oxo-2,3-dihydro-1H-benzo[d]imidazole-5-carboxylic acid

To a suspension of methyl 3-(1-(2,4-dichlorobenzoyl)azetidin-3-yl)-2-oxo-2,3-dihydro-1H-benzo[d]imidazole-5-carboxylate (30.4 mg, 0.0723 mmol) in methanol (5 mL) was added 1 N NaOH (723 μL, 0.723 mmol). the resulting mixture was stirred at 60° C. for 40 h. The solvent was removed under a stream of N₂, and the residue taken up in water and filtered through a syringe filter. The solution was acidified by addition of 6 N HCl and stirred for 18 h. The solid was collected by filtration, washed with water and dried under vacuum to give 3-(1-(2,4-dichlorobenzoyl)azetidin-3-yl)-2-oxo-2,3-dihydro-1H-benzo[d]imidazole-5-carboxylic acid (20.3 mg, 0.0500 mmol, 69.1% yield) as a white solid.

¹H NMR (400 MHz, DMSO-d₆) δ ppm 12.81 (br. s., 1H) 11.44 (s, 1H) 7.91 (s, 1H) 7.77 (dd, J=1.52, 0.76 Hz, 1H) 7.73 (dd, J=8.08, 1.52 Hz, 2H) 7.55 (d, J=1.52 Hz, 2H) 7.11 (d, J=8.08 Hz, 1H) 5.32-5.41 (m, 1H) 4.52 (d, J=7.07 Hz, 2H) 4.45 (dd, J=9.47, 5.43 Hz, 1H) 4.28 (t, J=9.22 Hz, 1H)

LCMS: [M+1]=406/408/410, rt=1.97 min.

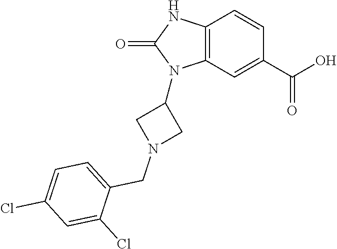 [SMILES: O=C(O)c1ccc2[nH]c(=O)n(C3CN(Cc4ccc(Cl)cc4Cl)C3)c2c1]

### Example 11: 3-(1-(2,4-dichlorobenzyl)azetidin-3-yl)-2-oxo-2,3-dihydro-1H-benzo[d]imidazole-5-carboxylic acid

### Methyl 3-(1-(2,4-dichlorobenzyl)azetidin-3-yl)-2-oxo-2,3-dihydro-1H-benzo[d]imidazole-5-carboxylate

A mixture of methyl 3-(azetidin-3-yl)-2-oxo-2,3-dihydro-1H-benzo[d]imidazole-5-carboxylate hydrochloride (40.0 mg, 0.141 mmol), 2,4-Dichlorobenzaldehyde (25.9 mg, 0.148 mmol) and Sodium triacetoxyborohydride (89.6 mg, 0.423 mmol) in chloroform (5 mL) was stirred at room temperature for 18 h. The contents were treated with 5% Na₂CO₃ and extracted with CHCl₃ (3×). The organic layers were filtered through a cotton plug, reduced in volume with a stream of N₂ and then purified by automated normal-phase chromatography (0-20% MeOH/DCM, 4 g silica gel cartridge) to give methyl 3-(1-(2,4-dichlorobenzyl)azetidin-3-yl)-2-oxo-2,3-dihydro-1H-benzo[d]imidazole-5-carboxylate (28.0 mg, 0.0689 mmol, 48.9% yield) as a white solid.

¹H NMR (400 MHz, DMSO-d₆) δ ppm 11.41 (br. s., 1H) 8.53 (s, 1H) 7.74 (d, J=8.08 Hz, 1H) 7.64 (d, J=2.02 Hz, 1H) 7.61 (d, J=8.59 Hz, 1H) 7.46 (dd, J=8.21, 2.15 Hz, 1H) 7.12 (d, J=8.08 Hz, 1H) 5.06 (d, J=6.57 Hz, 1H) 3.71-3.89 (m, 7H)

LCMS: [M+1]=406/408/410, rt=1.96 min.

### 3-(1-(2,4-dichlorobenzyl)azetidin-3-yl)-2-oxo-2,3-dihydro-1H-benzo[d]imidazole-5-carboxylic acid

To a suspension of methyl 3-(1-(2,4-dichlorobenzyl)azetidin-3-yl)-2-oxo-2,3-dihydro-1H-benzo[d]imidazole-5-carboxylate (28.0 mg, 0.0689 mmol) in methanol (5 mL) was added 1 N NaOH (689 μL, 0.689 mmol). the resulting mixture was stirred at 60° C. for 40 h. The solvent was removed under a stream of N₂, and the residue taken up in water and filtered through a syringe filter. The solution was acidified by addition of 6 N HCl and stirred for 18 h. The solid was collected by filtration, washed with water and dried under vacuum to give 3-(1-(2,4-dichlorobenzyl)azetidin-3-yl)-2-oxo-2,3-dihydro-1H-benzo[d]imidazole-5-carboxylic acid (23.5 mg, 0.0599 mmol, 86.9% yield) as a white solid.

¹H NMR (400 MHz, DMSO-d₆) δ ppm 12.71 (br. s., 1H) 11.35 (s, 1H) 8.46 (s, 1H) 7.71 (dd, J=8.08, 1.52 Hz, 1H) 7.64 (d, J=8.34 Hz, 1H) 7.61 (d, J=2.27 Hz, 1H) 7.43 (dd, J=8.34, 2.27 Hz, 1H) 7.09 (d, J=8.34 Hz, 1H) 4.99-5.08 (m, 1H) 3.87 (s, 2H) 3.72-3.81 (m, 4H)

LCMS: [M+1]=392/394/396, rt=1.79 min.

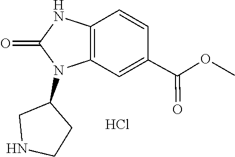 [SMILES: COC(=O)c1ccc2[nH]c(=O)n([C@H]3CCNC3)c2c1 ; Cl]

### Intermediate C: Methyl (S)-2-oxo-3-(pyrrolidin-3-yl)-2,3-dihydro-1H-benzo[d]imidazole-5-carboxylate hydrochloride

### tert-butyl (S)-3-((5-(methoxycarbonyl)-2-nitrophenyl)amino)pyrrolidine-1-carboxylate

A mixture of methyl 3-fluoro-4-nitrobenzoate (306 mg, 1.54 mmol), tert-butyl (S)-3-aminopyrrolidine-1-carboxylate (343 mg, 1.84 mmol) and cesium carbonate (601 mg, 1.84 mmol) in MeCN (5 mL) was stirred at 30° C. for 24 h, then at 60° C. for 12 h. The contents were taken up in EtOAc, washed with water (3×), brine (lx), dried over MgSO₄, filtered and the solvent removed in vacuo to give a residue which was purified by automated normal-phase chromatography (0-50% EtOAc/heptane, 20 g silica gel cartridge) to give tert-butyl (S)-3-((5-(methoxycarbonyl)-2-nitrophenyl)amino)pyrrolidine-1-carboxylate (400 mg, 1.095 mmol, 71.2% yield) as a yellow-orange foam.

¹H NMR (400 MHz, DMSO-d₆) δ ppm 8.19 (d, J=8.80 Hz, 1H) 7.89 (br. s., 1H) 7.57 (s, 1H) 7.22 (dd, J=8.88, 1.49 Hz, 1H) 4.37-4.49 (m, 1H) 3.90 (s, 3H) 3.68 (dd, J=9.98, 6.21 Hz, 1H) 3.35-3.45 (m, 2H) 3.26 (dd, J=10.93, 4.48 Hz, 1H) 2.25 (dd, J=12.65, 6.37 Hz, 1H) 1.93-2.07 (m, 1H) 1.41 (br. s., 9H)

LCMS: [M+23]=388, rt=1.70 min (lipophilic method).

### tert-butyl (S)-3-((2-amino-5-(methoxycarbonyl)phenyl)amino)pyrrolidine-1-carboxylate

To a solution of tert-butyl (S)-3-((5-(methoxycarbonyl)-2-nitrophenyl)amino)pyrrolidine-1-carboxylate (400 mg, 1.09 mmol) in ethyl acetate (25 mL) was added 10% Pd/C (40 mg, 0.109 mmol). The resulting suspension was stirred under a H₂ atmosphere (balloon) for 16 h, filtered through Celite and the solvent removed in vacuo to give tert-butyl (S)-3-((2-amino-5-(methoxycarbonyl)phenyl)amino)pyrrolidine-1-carboxylate (378 mg, 1.13 mmol, 103% yield) as a tan foam.

¹H NMR (400 MHz, DMSO-d₆) δ ppm 7.18 (dd, J=8.10, 1.81 Hz, 1H) 6.99 (s, 1H) 6.55 (d, J=8.17 Hz, 1H) 5.48-5.52 (m, 2H) 4.71 (d, J=5.66 Hz, 1H) 3.94-4.04 (m, 1H) 3.73 (s, 3H) 3.54-3.61 (m, 1H) 3.34-3.45 (m, 2H) 3.12-3.20 (m, 1H) 2.07-2.19 (m, 1H) 1.85 (td, J=12.18, 5.50 Hz, 1H) 1.40 (d, J=6.29 Hz, 9H)

LCMS: [M+1]=336, rt=1.21 min (lipophilic method).

### Methyl (S)-3-(1-(tert-butoxycarbonyl)pyrrolidin-3-yl)-2-oxo-2,3-dihydro-1H-benzo[d]imidazole-5-carboxylate

A mixture of tert-butyl (S)-3-((2-amino-5-(methoxycarbonyl)phenyl)amino)pyrrolidine-1-carboxylate (378 mg, 1.13 mmol) and 1,1′-carbonyldiimidazole (366 mg, 2.26 mmol) in chloroform (10 mL) was stirred at 60° C. for 4 h. The solvent was removed under a stream of N₂, and the residue triturated with MeOH/H₂O (1:1) for 65 h. The solid was collected by filtration, washed with MeOH/water (1:3) and dried to give methyl (S)-3-(1-(tert-butoxycarbonyl)pyrrolidin-3-yl)-2-oxo-2,3-dihydro-1H-benzo[d]imidazole-5-carboxylate (317 mg, 0.877 mmol, 77.8% yield) as an off-white solid.

¹H NMR (400 MHz, DMSO-d₆) δ ppm 11.42 (s, 1H) 7.68-7.74 (m, 2H) 7.10 (d, J 8.34 Hz, 1H) 5.07 (br. s., 1H) 3.83 (s, 3H) 3.52-3.70 (m, 3H) 2.44 (d, J=9.60 Hz, 2H) 2.15 (br. s., 1H) 1.43 (d, J=11.87 Hz, 9H)

LCMS: [M+23]=384, rt=2.28 min.

### Methyl (S)-2-oxo-3-(pyrrolidin-3-yl)-2,3-dihydro-1H-benzo[d]imidazole-5-carboxylate hydrochloride

To a suspension of methyl (S)-3-(1-(tert-butoxycarbonyl)pyrrolidin-3-yl)-2-oxo-2,3-dihydro-1H-benzo[d]imidazole-5-carboxylate (317 mg, 0.877 mmol) in methanol (10 mL) was added 6 N HCl (1.46 mL, 8.77 mmol). The resulting mixture was stirred at 60° C. for 90 min. The solvent was removed in vacuo to give methyl (S)-2-oxo-3-(pyrrolidin-3-yl)-2,3-dihydro-1H-benzo[d]imidazole-5-carboxylate hydrochloride (262 mg, 0.879 mmol, 100% yield) as a beige solid.

¹H NMR (400 MHz, DMSO-d₆) δ ppm 11.60 (s, 1H) 9.50 (br. s., 1H) 9.07 (br. s., 1H) 7.82 (d, J=1.26 Hz, 1H) 7.73 (dd, J=8.34, 1.52 Hz, 1H) 7.13 (d, J=8.34 Hz, 1H) 5.18 (dt, J=14.34, 7.11 Hz, 1H) 3.85 (s, 3H) 3.63-3.73 (m, 1H) 3.45-3.60 (m, 2H) 3.29 (dt, J=11.37, 5.94 Hz, 1H) 2.35 (q, J=7.33 Hz, 2H)

LCMS: [M+1]=262, rt=1.36 min.

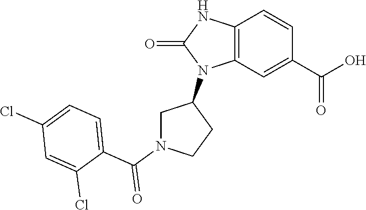 [SMILES: O=C(O)c1ccc2[nH]c(=O)n([C@H]3CCN(C(=O)c4ccc(Cl)cc4Cl)C3)c2c1]

### Example 12: (S)-3-(1-(2,4-dichlorobenzoyl)pyrrolidin-3-yl)-2-oxo-2,3-dihydro-1H-benzo[d]imidazole-5-carboxylic acid

### Methyl (S)-3-(1-(2,4-dichlorobenzoyl)pyrrolidin-3-yl)-2-oxo-2,3-dihydro-1H-benzo[d]imidazole-5-carboxylate

A mixture of methyl (S)-2-oxo-3-(pyrrolidin-3-yl)-2,3-dihydro-1H-benzo[d]imidazole-5-carboxylate hydrochloride (53.0 mg, 0.178 mmol), 2,4-dichlorobenzoic acid (37.4 mg, 0.196 mmol), HBTU (74.3 mg, 0.196 mmol) and DIPEA (124 μL, 0.712 mmol) in chloroform (5 mL) was stirred at room temperature for 21 h. The contents were treated with water and extracted with CHCl₃ (3×). The organic layers were filtered through a cotton plug, reduced in volume with a stream of N₂ and then purified by automated normal-phase chromatography (0-20% MeOH/DCM, 4 g silica gel cartridge) to give methyl (S)-3-(1-(2,4-dichlorobenzoyl)pyrrolidin-3-yl)-2-oxo-2,3-dihydro-1H-benzo[d]imidazole-5-carboxylate (65.0 mg, 0.150 mmol, 84.1% yield) as a beige foam. Two amide conformers seen by ¹H NMR—one peak by LCMS.

¹H NMR (400 MHz, DMSO-d₆) δ ppm 11.37-11.48 (m, 1H) 7.45-7.81 (m, 5H) 7.10 (dd, J=18.44, 8.34 Hz, 1H) 5.07-5.24 (m, 1H) 3.86-4.00 (m, 1H) 3.85 (d, J 2.78 Hz, 3H) 3.45-3.72 (m, 3H) 2.34 (br. s., 2H)

LCMS: [M+1]=434/436/438, rt=2.32 min.

### (S)-3-(1-(2,4-dichlorobenzoyl)pyrrolidin-3-yl)-2-oxo-2,3-dihydro-1H-benzo[d]imidazole-5-carboxylic acid

To a suspension of methyl (S)-3-(1-(2,4-dichlorobenzoyl)pyrrolidin-3-yl)-2-oxo-2,3-dihydro-1H-benzo[d]imidazole-5-carboxylate (65.0 mg, 0.150 mmol) in methanol (5 mL) was added 1 N NaOH (1.50 mL, 1.50 mmol). The resulting mixture was stirred at 60° C. for 3 h, then at 40° C. for 65 h. The solvent was removed under a stream of N₂, and the residue taken up in water and filtered through a syringe filter. The solution was acidified by addition of 6 N HCl and stirred for 18 h. The solid was collected by filtration, washed with water and dried under vacuum to give (S)-3-(1-(2,4-dichlorobenzoyl)pyrrolidin-3-yl)-2-oxo-2,3-dihydro-1H-benzo[d]imidazole-5-carboxylic acid (38.7 mg, 0.0921 mmol, 51.7% yield) as an off-white solid. Two amide conformers seen by ¹H NMR—one peak by LCMS.

¹H NMR (400 MHz, DMSO-d₆) δ ppm 12.78 (br. s., 1H) 11.31-11.42 (m, 1H) 7.46-7.80 (m, 5H) 7.07 (dd, J=18.70, 8.34 Hz, 1H) 5.05-5.24 (m, 1H) 3.38-4.00 (m, 4H) 2.15-2.46 (m, 2H)

LCMS: [M+1]=420/422/424, rt=2.00 min.

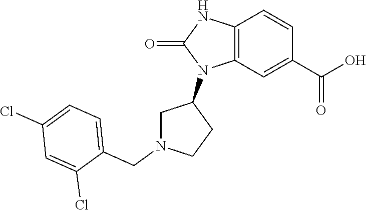 [SMILES: O=C(O)c1ccc2[nH]c(=O)n([C@H]3CCN(Cc4ccc(Cl)cc4Cl)C3)c2c1]

### Example 13: (S)-3-(1-(2,4-dichlorobenzyl)pyrrolidin-3-yl)-2-oxo-2,3-dihydro-1H-benzo[d]imidazole-5-carboxylic acid

### Methyl (S)-3-(1-(2,4-dichlorobenzyl)pyrrolidin-3-yl)-2-oxo-2,3-dihydro-1H-benzo[d]imidazole-5-carboxylate

A mixture of methyl (S)-2-oxo-3-(pyrrolidin-3-yl)-2,3-dihydro-1H-benzo[d]imidazole-5-carboxylate hydrochloride (53.0 mg, 0.178 mmol), 2,4-dichlorobenzaldehyde (34.3 mg, 0.196 mmol) and sodium triacetoxyborohydride (113 mg, 0.534 mmol) in chloroform (5 mL) was stirred at room temperature for 45 h. The contents were treated with 5% Na₂CO₃ and extracted with CHCl₃ (3×). The organic layers were filtered through a cotton plug, reduced in volume with a stream of N₂ and then purified by automated normal-phase chromatography (0-20% MeOH/DCM, 4 g silica gel cartridge) to give methyl (S)-3-(1-(2,4-dichlorobenzyl)pyrrolidin-3-yl)-2-oxo-2,3-dihydro-1H-benzo[d]imidazole-5-carboxylate (48.6 mg, 0.116 mmol, 65.0% yield) as a white foam.

¹H NMR (400 MHz, DMSO-d₆) δ ppm 11.34 (br. s., 1H) 8.32 (d, J=1.52 Hz, 1H) 7.78 (d, J=8.34 Hz, 1H) 7.71 (dd, J=8.21, 1.64 Hz, 1H) 7.62 (d, J=2.27 Hz, 1H) 7.41 (dd, J=8.34, 2.27 Hz, 1H) 7.08 (d, J=8.34 Hz, 1H) 5.05-5.15 (m, 1H) 3.85 (s, 3H) 3.78 (d, J=3.03 Hz, 2H) 3.15 (t, J=7.96 Hz, 1H) 3.00 (dd, J=10.36, 3.03 Hz, 1H) 2.70 (t, J=9.85 Hz, 1H) 2.32-2.42 (m, 1H) 2.17-2.28 (m, 1H) 1.95-2.07 (m, 1H)

LCMS: [M+1]=420/422/424, rt=2.00 min.

### (S)-3-(1-(2,4-dichlorobenzyl)pyrrolidin-3-yl)-2-oxo-2,3-dihydro-1H-benzo[d]imidazole-5-carboxylic acid

To a suspension of methyl (S)-3-(1-(2,4-dichlorobenzyl)pyrrolidin-3-yl)-2-oxo-2,3-dihydro-1H-benzo[d]imidazole-5-carboxylate (48.6 mg, 0.1200 mmol) in Methanol (5 mL) was added 1 N NaOH (1.16 mL, 1.16 mmol). The resulting mixture was stirred at 60° C. for 21 h. The solvent was removed under a stream of N₂, and the residue taken up in water and filtered through a syringe filter. The solution was acidified by addition of 6 N HCl and stirred for 18 h. The solid was collected by filtration, washed with water and dried under vacuum to give (S)-3-(1-(2,4-dichlorobenzyl)pyrrolidin-3-yl)-2-oxo-2,3-dihydro-1H-benzo[d]imidazole-5-carboxylic acid hydrochloride (32.9 mg, 0.0743 mmol, 64.3% yield) as a white solid.

¹H NMR (400 MHz, DMSO-d₆) δ ppm 12.82 (br. s., 1H) 11.67 (s, 1H) 11.52 (br. s., 1H) 11.03 (br. s., 1H) 10.21 (br. s., 1H) 7.57-7.92 (m, 5H) 7.07-7.15 (m, 1H) 5.10-5.48 (m, 2H) 4.62-4.79 (m, 2H) 3.57-3.98 (m, 4H)

LCMS: [M+1]=406/408/410, rt=1.83 min.

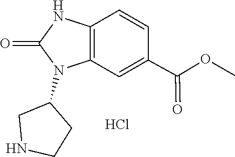 [SMILES: COC(=O)c1ccc2[nH]c(=O)n([C@@H]3CCNC3)c2c1 ; Cl]

### Intermediate D: Methyl (R)-2-oxo-3-(pyrrolidin-3-yl)-2,3-dihydro-1H-benzo[d]imidazole-5-carboxylate hydrochloride

### tert-butyl (R)-3-((5-(methoxycarbonyl)-2-nitrophenyl)amino)pyrrolidine-1-carboxylate

A mixture of methyl 3-fluoro-4-nitrobenzoate (298 mg, 1.50 mmol), tert-butyl (R)-3-aminopyrrolidine-1-carboxylate (334 mg, 1.80 mmol) and cesium carbonate (585 mg, 1.80 mmol) in MeCN (5 mL) was stirred at 50° C. for 17 h. The contents were taken up in EtOAc, washed with water (3×), brine (lx), dried over MgSO₄, filtered and the solvent removed in vacuo to give a residue which was purified by automated normal-phase chromatography (0-50% EtOAc/heptane, 20 g silica gel cartridge) to give tert-butyl (R)-3-((5-(methoxycarbonyl)-2-nitrophenyl)amino)pyrrolidine-1-carboxylate (340 mg, 0.931 mmol, 62.2% yield) as a yellow-orange gum.

¹H NMR (400 MHz, DMSO-d₆) δ ppm 8.20 (d, J=8.80 Hz, 1H) 7.89 (br. s., 1H) 7.58 (s, 1H) 7.22 (dd, J=8.80, 1.57 Hz, 1H) 4.43 (d, J=16.66 Hz, 1H) 3.90 (s, 3H) 3.64-3.71 (m, 1H) 3.35-3.45 (m, 2H) 3.26 (dd, J=11.00, 4.40 Hz, 1H) 2.19-2.30 (m, 1H) 1.94-2.08 (m, 1H) 1.41 (br. s., 9H)

LCMS: [M+23]=388, rt=1.70 min (lipophilic method).

### tert-butyl (R)-3-((2-amino-5-(methoxycarbonyl)phenyl)amino)pyrrolidine-1-carboxylate

To a solution of tert-butyl (R)-3-((5-(methoxycarbonyl)-2-nitrophenyl)amino)pyrrolidine-1-carboxylate (340 mg, 0.931 mmol) in ethyl acetate (25 mL) was added 10% Pd/C (35.0 mg, 0.093 mmol). The resulting suspension was stirred under a H₂ atmosphere (balloon) for 18 h. The contents were filtered through Celite and the solvent removed in vacuo to give tert-butyl (R)-3-((2-amino-5-(methoxycarbonyl)phenyl)amino)pyrrolidine-1-carboxylate (332 mg, 0.990 mmol, 106% yield) as a tan foam. This material was used without further purification.

¹H NMR (400 MHz, DMSO-d₆) δ ppm 7.18 (dd, J=8.17, 1.73 Hz, 1H) 6.99 (s, 1H) 6.55 (d, J=8.17 Hz, 1H) 5.50 (s, 2H) 4.71 (d, J=5.66 Hz, 1H) 3.94-4.05 (m, 1H) 3.73 (s, 3H) 3.53-3.61 (m, 1H) 3.34-3.45 (m, 2H) 3.16 (t, J=11.71 Hz, 1H) 2.08-2.19 (m, 1H) 1.85 (td, J=12.42, 5.19 Hz, 1H) 1.40 (d, J=5.97 Hz, 9H)

LCMS: [M+23]=358, rt=1.21 min (lipophilic method).

### Methyl (R)-3-(1-(tert-butoxycarbonyl)pyrrolidin-3-yl)-2-oxo-2,3-dihydro-1H-benzo[d]imidazole-5-carboxylate

A mixture of tert-butyl (R)-3-((2-amino-5-(methoxycarbonyl)phenyl)amino)pyrrolidine-1-carboxylate (332 mg, 0.990 mmol) and 1,1′-carbonyldiimidazole (321 mg, 1.98 mmol) in chloroform (10 mL) was stirred at 60° C. for 4 h. The solvent was removed under a stream of N₂, and the residue triturated with MeOH/H2O (1:1) for 65 h. The solid was collected by filtration, washed with MeOH/water (1:3) and dried to give methyl (R)-3-(1-(tert-butoxycarbonyl)pyrrolidin-3-yl)-2-oxo-2,3-dihydro-1H-benzo[d]imidazole-5-carboxylate (284 mg, 0.787 mmol, 79.5% yield) as an off-white solid.

¹H NMR (400 MHz, DMSO-d₆) δ ppm 11.42 (br. s., 1H) 7.67-7.75 (m, 2H) 7.10 (d, J 8.34 Hz, 1H) 5.06 (br. s., 1H) 3.84 (s, 3H) 3.51-3.71 (m, 3H) 2.34 (br. s., 2H) 2.16 (br. s., 1H) 1.43 (d, J=11.12 Hz, 9H)

LCMS: [M+23]=384, rt=2.28 min.

### Methyl (R)-2-oxo-3-(pyrrolidin-3-yl)-2,3-dihydro-1H-benzo[d]imidazole-5-carboxylate hydrochloride

To a suspension of methyl (R)-3-(1-(tert-butoxycarbonyl)pyrrolidin-3-yl)-2-oxo-2,3-dihydro-1H-benzo[d]imidazole-5-carboxylate (284 mg, 0.7987 mmol) in methanol (10 mL) was added 6 N HCl (1.31 mL, 7.86 mmol). The resulting mixture was stirred at 60° C. for 90 min. The solvent was removed in vacuo to give methyl (R)-2-oxo-3-(pyrrolidin-3-yl)-2,3-dihydro-1H-benzo[d]imidazole-5-carboxylate hydrochloride (229 mg, 0.768 mmol, 97.6% yield) as a beige solid.

¹H NMR (400 MHz, DMSO-d₆) δ ppm 11.61 (s, 1H) 9.53 (br. s., 1H) 9.09 (br. s., 1H) 7.83 (s, 1H) 7.74 (dd, J=8.34, 1.52 Hz, 1H) 7.13 (d, J=8.08 Hz, 1H) 5.12-5.24 (m, 1H) 3.86 (s, 3H) 3.67 (br. s., 1H) 3.52 (br. s., 2H) 3.29 (br. s., 1H) 2.35 (q, J=7.24 Hz, 2H)

LCMS: [M+1]=262, rt=1.36 min.

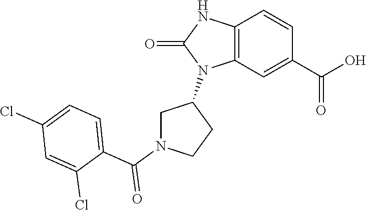 [SMILES: O=C(O)c1ccc2[nH]c(=O)n([C@@H]3CCN(C(=O)c4ccc(Cl)cc4Cl)C3)c2c1]

### Example 14: (R)-3-(1-(2,4-dichlorobenzoyl)pyrrolidin-3-yl)-2-oxo-2,3-dihydro-1H-benzo[d]imidazole-5-carboxylic acid

### Methyl (R)-3-(1-(2,4-dichlorobenzoyl)pyrrolidin-3-yl)-2-oxo-2,3-dihydro-1H-benzo[d]imidazole-5-carboxylate

A mixture of methyl (R)-2-oxo-3-(pyrrolidin-3-yl)-2,3-dihydro-1H-benzo[d]imidazole-5-carboxylate hydrochloride (53.0 mg, 0.178 mmol), 2,4-dichlorobenzoic acid (37.4 mg, 0.196 mmol), HBTU (74.3 mg, 0.196 mmol) and DIPEA (124 μL, 0.712 mmol) in chloroform (5 mL) was stirred at room temperature for 21 h. The contents were treated with water and extracted with CHCl₃ (3×). The organic layers were filtered through a cotton plug, reduced in volume with a stream of N₂ and then purified by automated normal-phase chromatography (0-20% MeOH/DCM, 4 g silica gel cartridge) to give methyl (R)-3-(1-(2,4-dichlorobenzoyl)pyrrolidin-3-yl)-2-oxo-2,3-dihydro-1H-benzo[d]imidazole-5-carboxylate (60.0 mg, 0.138 mmol, 77.6% yield) as a white foam. Two amide conformers seen by 1H NMR—one peak by LCMS.

¹H NMR (400 MHz, DMSO-d₆) δ ppm 11.37-11.47 (m, 1H) 7.45-7.81 (m, 5H) 7.10 (dd, J=18.70, 8.34 Hz, 1H) 5.06-5.24 (m, 1H) 3.87-4.01 (m, 1H) 3.85 (d, J 2.78 Hz, 3H) 3.38-3.72 (m, 3H) 2.16-2.35 (m, 2H)

LCMS: [M+1]=434/436/468, rt=2.32 min.

### (R)-3-(1-(2,4-dichlorobenzoyl)pyrrolidin-3-yl)-2-oxo-2,3-dihydro-1H-benzo[d]imidazole-5-carboxylic acid

To a suspension of methyl (R)-3-(1-(2,4-dichlorobenzoyl)pyrrolidin-3-yl)-2-oxo-2,3-dihydro-1H-benzo[d]imidazole-5-carboxylate (60.0 mg, 0.138 mmol) in methanol (5 mL) was added 1 N NaOH (1.38 mL, 1.38 mmol). The resulting mixture was stirred at 60° C. for 3 h, then at 40° C. for 65 h. The solvent was removed under a stream of N₂, and the residue taken up in water and filtered through a syringe filter. The solution was acidified by addition of 6 N HCl and stirred for 18 h. The solid was collected by filtration, washed with water and dried under vacuum to give (R)-3-(1-(2,4-dichlorobenzoyl)pyrrolidin-3-yl)-2-oxo-2,3-dihydro-1H-benzo[d]imidazole-5-carboxylic acid (39.6 mg, 0.0942 mmol, 68.2% yield) as an off-white solid. Two amide conformers by 1H NMR—one peak by LCMS.

¹H NMR (400 MHz, DMSO-d₆) δ ppm 12.77 (br. s., 1H) 11.31-11.41 (m, 1H) 7.46-7.80 (m, 5H) 7.07 (dd, J=18.57, 8.21 Hz, 1H) 5.05-5.24 (m, 1H) 3.39-4.01 (m, 4H) 2.14-2.43 (m, 2H)

LCMS: [M+1]=420/422/424, rt=2.00 min.

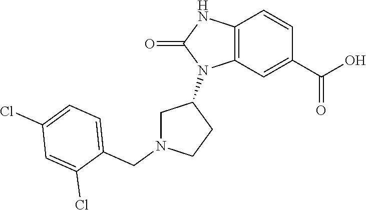 [SMILES: O=C(O)c1ccc2[nH]c(=O)n([C@@H]3CCN(Cc4ccc(Cl)cc4Cl)C3)c2c1]

### Example 15: (R)-3-(1-(2,4-dichlorobenzyl)pyrrolidin-3-yl)-2-oxo-2,3-dihydro-1H-benzo[d]imidazole-5-carboxylic acid

### Methyl (R)-3-(1-(2,4-dichlorobenzyl)pyrrolidin-3-yl)-2-oxo-2,3-dihydro-1H-benzo[d]imidazole-5-carboxylate

A mixture of methyl (R)-2-oxo-3-(pyrrolidin-3-yl)-2,3-dihydro-1H-benzo[d]imidazole-5-carboxylate hydrochloride (53.0 mg, 0.178 mmol), 2,4-dichlorobenzaldehyde (34.3 mg, 0.196 mmol) and sodium triacetoxyborohydride (113 mg, 0.534 mmol) in chloroform (5 mL) was stirred at room temperature for 45 h. The contents were treated with 5% Na₂CO₃ and extracted with CHCl₃ (3×). The organic layers were filtered through a cotton plug, reduced in volume with a stream of N₂ and then purified by automated normal-phase chromatography (0-20% MeOH/DCM, 4 g silica gel cartridge) to give methyl (R)-3-(1-(2,4-dichlorobenzyl)pyrrolidin-3-yl)-2-oxo-2,3-dihydro-1H-benzo[d]imidazole-5-carboxylate (46.0 mg, 0.109 mmol, 61.5% yield) as a white foam.

¹H NMR (400 MHz, DMSO-d₆) δ ppm 11.34 (br. s., 1H) 8.32 (d, J=1.52 Hz, 1H) 7.78 (d, J=8.34 Hz, 1H) 7.71 (dd, J=8.34, 1.52 Hz, 1H) 7.62 (d, J=2.27 Hz, 1H) 7.41 (dd, J=8.34, 2.27 Hz, 1H) 7.08 (d, J=8.34 Hz, 1H) 5.05-5.15 (m, 1H) 3.85 (s, 3H) 3.78 (d, J=3.03 Hz, 2H) 3.15 (t, J=7.96 Hz, 1H) 3.00 (dd, J=10.36, 3.03 Hz, 1H) 2.70 (t, J=9.73 Hz, 1H) 2.32-2.42 (m, 1H) 2.17-2.28 (m, 1H) 1.94-2.07 (m, 1H)

LCMS: [M+1]=420/422/424, rt=1.99 min.

### (R)-3-(1-(2,4-dichlorobenzyl)pyrrolidin-3-yl)-2-oxo-2,3-dihydro-1H-benzo[d]imidazole-5-carboxylic acid

To a suspension of methyl (R)-3-(1-(2,4-dichlorobenzyl)pyrrolidin-3-yl)-2-oxo-2,3-dihydro-1H-benzo[d]imidazole-5-carboxylate (46.0 mg, 0.109 mmol) in methanol (5 mL) was added 1 N NaOH (1.09 mL, 1.09 mmol). The resulting mixture was stirred at 60° C. for 21 h. The solvent was removed under a stream of N₂, and the residue taken up in water and filtered through a syringe filter. The solution was acidified by addition of 6 N HCl and stirred for 18 h. The solid was collected by filtration, washed with water and dried under vacuum to give (R)-3-(1-(2,4-dichlorobenzyl)pyrrolidin-3-yl)-2-oxo-2,3-dihydro-1H-benzo[d]imidazole-5-carboxylic acid hydrochloride (27.2 mg, 0.0614 mmol, 56.1% yield) as an off-white solid.

¹H NMR (400 MHz, DMSO-d₆) δ ppm 12.81 (br. s., 1H) 11.67 (s, 1H) 11.52 (br. s., 1H) 10.92 (br. s., 1H) 10.17 (br. s., 1H) 7.58-7.90 (m, 5H) 7.07-7.15 (m, 1H) 5.43 (br. s., 1H) 5.16 (br. s., 1H) 4.73 (br. s., 1H) 4.65 (br. s., 1H) 3.57-3.98 (m, 4H)

LCMS: [M+1]=406/408/410, rt=1.82 min.

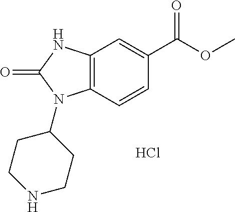 [SMILES: COC(=O)c1ccc2c(c1)[nH]c(=O)n2C1CCNCC1 ; Cl]

### Intermediate E: Methyl 2-oxo-1-(piperidin-4-yl)-2,3-dihydro-1H-benzo[d]imidazole-5-carboxylate hydrochloride

### tert-butyl 4-((4-(methoxycarbonyl)-2-nitrophenyl)amino)piperidine-1-carboxylate

A mixture of methyl 4-chloro-3-nitrobenzoate (325 mg, 1.51 mmol), tert-butyl 4-aminopiperidine-1-carboxylate (362 mg, 1.81 mmol) and cesium carbonate (589 mg, 1.81 mmol) in MeCN (8 mL) was stirred at room temperature for 17 h, then at 50° C. for 26 h, then at 30° C. for 3 days. The contents were taken up in EtOAc, washed with water (3×), brine (lx), dried over MgSO₄, filtered and the solvent removed in vacuo to give a residue which was purified by automated normal-phase chromatography (0-100% EtOAc/heptane, 20 g silica gel cartridge) to give tert-butyl 4-((4-(methoxycarbonyl)-2-nitrophenyl)amino)piperidine-1-carboxylate (340 mg, 0.896 mmol, 59.4% yield) as a yellow solid.

¹H NMR (400 MHz, DMSO-d₆) δ ppm 8.63 (d, J=2.04 Hz, 1H) 8.24 (d, J=8.02 Hz, 1H) 7.98 (dd, J=9.12, 2.04 Hz, 1H) 7.29 (d, J=9.27 Hz, 1H) 3.92 (dd, J 7.62, 3.38 Hz, 3H) 3.83 (s, 3H) 2.96 (br. s., 2H) 1.94 (d, J=10.22 Hz, 2H) 1.51 (q, J=10.53 Hz, 2H) 1.41 (s, 9H)

LCMS: [M+23]=402, rt=1.80 min (lipophilic method).

### tert-butyl 4-((2-amino-4-(methoxycarbonyl)phenyl)amino)piperidine-1-carboxylate

To a solution of tert-butyl 4-((4-(methoxycarbonyl)-2-nitrophenyl)amino)piperidine-1-carboxylate (340 mg, 0.896 mmol) in ethyl acetate (25 mL) was added 10% Pd/C (35 mg, 0.090 mmol). The resulting mixture was stirred under atmospheric H₂ (balloon) for 6 days (for convenience). The contents were filtered through Celite and the solvent removed in vacuo to give tert-butyl 4-((2-amino-4-(methoxycarbonyl)phenyl)amino)piperidine-1-carboxylate (325 mg, 0.929 mmol, 103% yield) as a greenish-gray foam. This material was sued without further purification.

¹H NMR (400 MHz, DMSO-d₆) δ ppm 7.16-7.20 (m, 2H) 6.53 (d, J=8.17 Hz, 1H) 4.98 (d, J=7.55 Hz, 1H) 4.77 (br. s., 2H) 3.92 (d, J=11.16 Hz, 2H) 3.72 (s, 3H) 3.49-3.58 (m, 1H) 2.91 (br. s., 2H) 1.87-1.96 (m, 2H) 1.41 (s, 9H) 1.23-1.34 (m, 2H)

LCMS: [M+1]=350, rt=1.14 min (lipophilic method).

### Methyl 1-(1-(tert-butoxycarbonyl)piperidin-4-yl)-2-oxo-2,3-dihydro-1H-benzo[d]imidazole-5-carboxylate

A mixture of tert-butyl 4-((2-amino-4-(methoxycarbonyl)phenyl)amino)piperidine-1-carboxylate (310 mg, 0.887 mmol) and 1,1′-carbonyldiimidazole (216 mg, 1.33 mmol) in chloroform (10 mL) was stirred at 60° C. for 21 h. The reaction mixture was reduced in volume with a stream of N₂ and purified by automated normal-phase chromatography (0-100% EtOAc/heptane, 20 g silica gel cartridge) to give methyl 1-(1-(tert-butoxycarbonyl)piperidin-4-yl)-2-oxo-2,3-dihydro-1H-benzo[d]imidazole-5-carboxylate (255 mg, 0.678 mmol, 76.5% yield) as a gray foam.

¹H NMR (400 MHz, DMSO-d₆) δ ppm 11.17 (s, 1H) 7.68 (dd, J=8.33, 1.57 Hz, 1H) 7.51 (d, J=1.57 Hz, 1H) 7.35 (d, J=8.33 Hz, 1H) 4.33-4.42 (m, 1H) 4.10 (br. s., 2H) 3.83 (s, 3H) 2.88 (br. s., 2H) 2.19 (qd, J=12.50, 4.48 Hz, 2H) 1.71 (d, J 10.38 Hz, 2H) 1.44 (s, 9H)

LCMS: [M+23]=398, rt=1.26 min (lipophilic method).

### Methyl 2-oxo-1-(piperidin-4-yl)-2,3-dihydro-1H-benzo[d]imidazole-5-carboxylate hydrochloride

To a suspension of methyl 1-(1-(tert-butoxycarbonyl)piperidin-4-yl)-2-oxo-2,3-dihydro-1H-benzo[d]imidazole-5-carboxylate (255 mg, 0.678 mmol) in methanol (5 mL) was added 6 N HCl (1.13 mL, 6.77 mmol). The resulting mixture was stirred at 60° C. for 4 h. The solvent was removed in vacuo to give methyl 2-oxo-1-(piperidin-4-yl)-2,3-dihydro-1H-benzo[d]imidazole-5-carboxylate hydrochloride (191 mg, 0.614 mmol, 90.7% yield) as a beige solid.

¹H NMR (400 MHz, DMSO-d₆) δ ppm 11.26 (s, 1H) 8.77-8.93 (m, 2H) 7.71 (dd, J 8.41, 1.65 Hz, 1H) 7.54 (d, J=1.41 Hz, 1H) 7.52 (d, J=8.49 Hz, 1H) 4.53-4.62 (m, 1H) 3.84 (s, 3H) 3.42 (d, J=12.42 Hz, 2H) 3.09 (q, J=11.69 Hz, 2H) 2.55-2.66 (m, 2H) 1.88 (d, J=12.26 Hz, 2H)

LCMS: [M+1]=276, rt=1.41 min

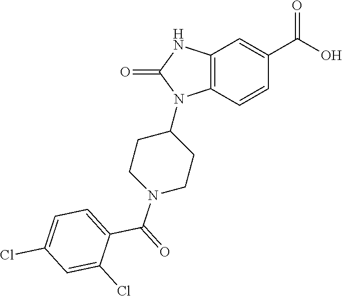 [SMILES: O=C(O)c1ccc2c(c1)[nH]c(=O)n2C1CCN(C(=O)c2ccc(Cl)cc2Cl)CC1]

### Example 16: 1-(1-(2,4-dichlorobenzoyl)piperidin-4-yl)-2-oxo-2,3-dihydro-1H-benzo[d]imidazole-5-carboxylic acid

### Methyl 1-(1-(2,4-dichlorobenzoyl)piperidin-4-yl)-2-oxo-2,3-dihydro-1H-benzo[d]imidazole-5-carboxylate

A mixture of methyl 2-oxo-1-(piperidin-4-yl)-2,3-dihydro-1H-benzo[d]imidazole-5-carboxylate hydrochloride (50.0 mg, 0.160 mmol), 2,4-dichlorobenzoic acid (33.7 mg, 0.176 mmol), HBTU (66.9 mg, 0.176 mmol) and DIPEA (112 μL, 0.642 mmol) in chloroform (5 mL) was stirred at room temperature for 18 h. The contents were treated with water and extracted with CHCl₃ (3×). The organic layers were filtered through a cotton plug, reduced in volume with a stream of N₂ and then purified by automated normal-phase chromatography (0-20% MeOH/DCM, 4 g silica gel cartridge) to give methyl 1-(1-(2,4-dichlorobenzoyl)piperidin-4-yl)-2-oxo-2,3-dihydro-1H-benzo[d]imidazole-5-carboxylate (49.3 mg, 0.110 mmol, 68.6% yield) as a white foam. Two amide conformers by ¹H NMR—one peak by LCMS.

¹H NMR (400 MHz, DMSO-d₆) δ ppm 11.23 (d, J=8.34 Hz, 1H) 7.25-7.80 (m, 6H) 4.47-4.73 (m, 2H) 3.84 (s, 3H) 3.24 (d, J=16.67 Hz, 1H) 2.89-3.00 (m, 1H) 2.08-2.40 (m, 2H) 1.61-1.91 (m, 2H)

LCMS: [M+1]=448/450/452, rt=2.41 min.

### 1-(1-(2,4-dichlorobenzoyl)piperidin-4-yl)-2-oxo-2,3-dihydro-1H-benzo[d]imidazole-5-carboxylic acid

To a suspension of methyl 1-(1-(2,4-dichlorobenzoyl)piperidin-4-yl)-2-oxo-2,3-dihydro-1H-benzo[d]imidazole-5-carboxylate (49.3 mg, 0.110 mmol) in methanol (5 mL) was added 1 N NaOH (1.10 mL, 1.10 mmol). The resulting mixture was stirred at 60° C. for 3 h, then at 40° C. for 65 h, then at 60° C. for 2 h. The solvent was removed under a stream of N₂, and the residue taken up in water and filtered through a syringe filter. The solution was acidified by addition of 6 N HCl and stirred for 18 h. The solid was collected by filtration, washed with water and dried under vacuum to give 1-(1-(2,4-dichlorobenzoyl)piperidin-4-yl)-2-oxo-2,3-dihydro-1H-benzo[d]imidazole-5-carboxylic acid (33.5 mg, 0.0771 mmol, 70.1% yield) as a white solid. Two amide conformers seen by ¹H NMR—one peak by LCMS.

¹H NMR (400 MHz, DMSO-d₆) δ ppm 12.71 (br. s., 1H) 11.17 (d, J=7.58 Hz, 1H) 7.21-7.80 (m, 6H) 4.69 (d, J=11.87 Hz, 1H) 4.46-4.59 (m, 1H) 3.17-3.41 (m, 2H) 2.90-3.01 (m, 1H) 2.11-2.39 (m, 2H) 1.61-1.93 (m, 2H)

LCMS: [M+1]=434/436/438, rt=2.13 min.

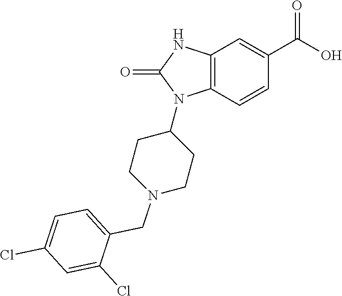 [SMILES: O=C(O)c1ccc2c(c1)[nH]c(=O)n2C1CCN(Cc2ccc(Cl)cc2Cl)CC1]

### Example 17: 1-(1-(2,4-dichlorobenzyl)piperidin-4-yl)-2-oxo-2,3-dihydro-1H-benzo[d]imidazole-5-carboxylic acid

### Methyl 1-(1-(2,4-dichlorobenzyl)piperidin-4-yl)-2-oxo-2,3-dihydro-1H-benzo[d]imidazole-5-carboxylate

A mixture of methyl 2-oxo-1-(piperidin-4-yl)-2,3-dihydro-1H-benzo[d]imidazole-5-carboxylate hydrochloride (50.0 mg, 0.160 mmol), 2,4-dichlorobenzaldehyde (30.9 mg, 0.176 mmol) and sodium triacetoxyborohydride (119 mg, 0.561 mmol) in chloroform (5 mL) was stirred at room temperature for 41 h. The contents were treated with water and extracted with CHCl₃ (3×). The organic layers were filtered through a cotton plug, reduced in volume with a stream of N₂ and then purified by automated normal-phase chromatography (0-20% MeOH/DCM, 4 g silica gel cartridge) to give methyl 1-(1-(2,4-dichlorobenzyl)piperidin-4-yl)-2-oxo-2,3-dihydro-1H-benzo[d]imidazole-5-carboxylate (36.6 mg, 0.0843 mmol, 52.5% yield) as a white foam.

¹H NMR (400 MHz, DMSO-d₆) δ ppm 11.20 (s, 1H) 7.70 (dd, J=8.34, 1.77 Hz, 1H) 7.58-7.63 (m, 2H) 7.51 (d, J=1.52 Hz, 1H) 7.46 (dd, J=8.34, 2.02 Hz, 1H) 7.38 (d, J=8.34 Hz, 1H) 4.16-4.27 (m, 1H) 3.83 (s, 3H) 3.61 (s, 2H) 2.96 (d, J=10.86 Hz, 2H) 2.31-2.41 (m, 2H) 2.19-2.28 (m, 2H) 1.69 (d, J=11.62 Hz, 2H)

LCMS: [M+1]=434/436/438, rt=2.00 min.

### 1-(1-(2,4-dichlorobenzyl)piperidin-4-yl)-2-oxo-2,3-dihydro-1H-benzo[d]imidazole-5-carboxylic acid

To a suspension of methyl 1-(1-(2,4-dichlorobenzyl)piperidin-4-yl)-2-oxo-2,3-dihydro-1H-benzo[d]imidazole-5-carboxylate (36.6 mg, 0.0843 mmol) in methanol (5 mL) was added 1 N NaOH (843 μL, 0.843 mmol). The resulting mixture was stirred at 60° C. for 21 h. The solvent was removed under a stream of N₂, and the residue taken up in water and filtered through a syringe filter. The solution was acidified by addition of 6 N HCl and stirred for 18 h. The solid was collected by filtration, washed with water and dried under vacuum to give 1-(1-(2,4-dichlorobenzyl)piperidin-4-yl)-2-oxo-2,3-dihydro-1H-benzo[d]imidazole-5-carboxylic acid (25.9 mg, 0.0616 mmol, 73.1% yield) as a white solid.

¹H NMR (400 MHz, DMSO-d₆) δ ppm 12.69 (br. s., 1H) 11.14 (s, 1H) 7.68 (dd, J 8.34, 1.77 Hz, 1H) 7.59-7.63 (m, 2H) 7.50 (d, J=1.77 Hz, 1H) 7.46 (dd, J=8.21, 2.15 Hz, 1H) 7.34 (d, J=8.59 Hz, 1H) 4.21 (t, J=12.13 Hz, 1H) 3.61 (s, 2H) 2.96 (d, J=11.12 Hz, 2H) 2.32-2.44 (m, 2H) 2.18-2.28 (m, 2H) 1.68 (d, J=11.37 Hz, 2H)

LCMS: [M+1]=420/422/424, rt=1.83 min.

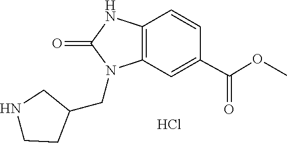 [SMILES: COC(=O)c1ccc2[nH]c(=O)n(CC3CCNC3)c2c1 ; Cl]

### Intermediate F: Methyl 2-oxo-3-(pyrrolidin-3-ylmethyl)-2,3-dihydro-1H-benzo[d]imidazole-5-carboxylate hydrochloride

### tert-butyl 3-(((5-(methoxycarbonyl)-2-nitrophenyl)amino)methyl)pyrrolidine-1-carboxylate

A mixture of methyl 3-fluoro-4-nitrobenzoate (300 mg, 1.51 mmol), tert-butyl 3-(aminomethyl)pyrrolidine-1-carboxylate (362 mg, 1.81 mmol) and cesium carbonate (589 mg, 1.81 mmol) in MeCN (5 mL) was stirred at 70° C. for 2 h, then at 40° C. for 65 h. The contents were taken up in EtOAc, washed with water (3×), brine (1×), dried over MgSO₄, filtered and the solvent removed in vacuo to give a residue which was purified by automated normal-phase chromatography (0-50% EtOAc/heptane, 20 g silica gel cartridge) to give tert-butyl 3-(((5-(methoxycarbonyl)-2-nitrophenyl)amino)methyl)pyrrolidine-1-carboxylate (530 mg, 1.40 mmol, 92.7% yield) as a tacky orange solid.

¹H NMR (400 MHz, DMSO-d₆) δ ppm 8.16-8.22 (m, 2H) 7.55 (d, J=1.52 Hz, 1H) 7.17 (dd, J=8.84, 1.52 Hz, 1H) 3.89 (s, 3H) 3.37-3.51 (m, 4H) 3.17-3.29 (m, 1H) 3.01-3.10 (m, 1H) 2.53-2.62 (m, 1H) 1.91-2.04 (m, 1H) 1.67 (td, J=13.26, 7.83 Hz, 1H) 1.39 (s, 9H)

LCMS: [M+23]=402, rt=1.72 min (lipophilic method).

### tert-butyl 3-(((2-amino-5-(methoxycarbonyl)phenyl)amino)methyl)pyrrolidine-1-carboxylate

To a solution of tert-butyl 3-(((5-(methoxycarbonyl)-2-nitrophenyl)amino)methyl)pyrrolidine-1-carboxylate (530 mg, 1.40 mmol) in ethyl acetate (25 mL) was added 10% Pd/C (60 mg, 0.14 mmol). The resulting suspension was stirred under a H₂ atmosphere (balloon) for 6 days (for convenience). The contents were filtered through Celite and the solvent removed in vacuo to give tert-butyl 3-(((2-amino-5-(methoxycarbonyl)phenyl)amino)methyl)pyrrolidine-1-carboxylate (502 mg, 1.44 mmol, 102% yield) as a tan foam.

¹H NMR (400 MHz, DMSO-d₆) δ ppm 7.16 (dd, J=8.08, 2.02 Hz, 1H) 6.96 (d, J 2.02 Hz, 1H) 6.54 (d, J=8.08 Hz, 1H) 5.51 (s, 2H) 4.63 (br. s., 1H) 3.73 (s, 3H) 3.47-3.56 (m, 1H) 3.32-3.41 (m, 1H) 3.17-3.29 (m, 1H) 2.98-3.07 (m, 3H) 2.39-2.48 (m, 1H) 2.04 (br. s., 1H) 1.67 (td, J=13.07, 8.46 Hz, 1H) 1.40 (s, 9H)

LCMS: [M+23]=372, rt=1.17 min (lipophilic method).

### Methyl 3-((1-(tert-butoxycarbonyl)pyrrolidin-3-yl)methyl)-2-oxo-2,3-dihydro-1H-benzo[d]imidazole-5-carboxylate

A mixture of tert-butyl 3-(((2-amino-5-(methoxycarbonyl)phenyl)amino)methyl)pyrrolidine-1-carboxylate (502 mg, 1.44 mmol) and 1,1′-carbonyldiimidazole (419 mg, 2.59 mmol) in chloroform (10 mL) was stirred at 60° C. for 20 h. After cooling, the solvent volume was reduced with a stream of N₂. The residue was triturated with water/MeOH (2:1) for 18 h. The solid was collected by filtration, washed with water and dried under vacuum to give methyl 3-((1-(tert-butoxycarbonyl)pyrrolidin-3-yl)methyl)-2-oxo-2,3-dihydro-1H-benzo[d]imidazole-5-carboxylate (447 mg, 1.19 mmol, 82.9% yield) as a beige solid.

¹H NMR (400 MHz, DMSO-d₆) δ ppm 11.37 (br. s., 1H) 7.75 (d, J=1.26 Hz, 1H) 7.70 (dd, J=8.21, 1.64 Hz, 1H) 7.10 (d, J=8.08 Hz, 1H) 3.89 (d, J=4.80 Hz, 2H) 3.84 (s, 3H) 3.28-3.35 (m, 2H) 3.14-3.25 (m, 1H) 2.98-3.10 (m, 1H) 2.57-2.67 (m, 1H) 1.80-1.91 (m, 1H) 1.57-1.69 (m, 1H) 1.38 (d, J=5.81 Hz, 9H)

LCMS: [M−100+1]=276, rt=2.27 min (lipophilic method).

### Methyl 2-oxo-3-(pyrrolidin-3-ylmethyl)-2,3-dihydro-1H-benzo[d]imidazole-5-carboxylate hydrochloride

To a suspension of methyl 3-((1-(tert-butoxycarbonyl)pyrrolidin-3-yl)methyl)-2-oxo-2,3-dihydro-1H-benzo[d]imidazole-5-carboxylate (447 mg, 1.19 mmol) in methanol (10 mL) was added 6 N HCl (1.98 mL, 11.9 mmol). The resulting mixture was stirred at 60° C. for 1 h, then allowed to cool to room temperature with stirring overnight. The solvent volume was reduced with a stream of N₂, then removed under vacuum to give methyl 2-oxo-3-(pyrrolidin-3-ylmethyl)-2,3-dihydro-1H-benzo[d]imidazole-5-carboxylate hydrochloride (362 mg, 1.16 mmol, 97.5% yield) as a tan foam.

¹H NMR (400 MHz, DMSO-d₆) δ ppm 11.45 (s, 1H) 9.09 (br. s., 2H) 7.81 (d, J 1.26 Hz, 1H) 7.72 (dd, J=8.34, 1.52 Hz, 1H) 7.11 (d, J=8.08 Hz, 1H) 3.97 (dd, J 7.20, 2.15 Hz, 2H) 3.85 (s, 3H) 3.20-3.33 (m, 2H) 3.03-3.15 (m, 1H) 2.89-3.00 (m, 1H) 2.73 (dt, J=15.28, 7.52 Hz, 1H) 1.92-2.03 (m, 1H) 1.67 (dq, J=12.88, 8.42 Hz, 1H)

LCMS: [M+1]=276, rt=1.36 min.

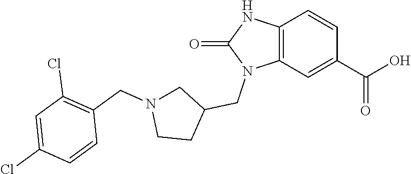 [SMILES: O=C(O)c1ccc2[nH]c(=O)n(CC3CCN(Cc4ccc(Cl)cc4Cl)C3)c2c1]

### Example 18: 3-((1-(2,4-dichlorobenzyl)pyrrolidin-3-yl)methyl)-2-oxo-2,3-dihydro-1H-benzo[d]imidazole-5-carboxylic acid

### Methyl 3-((1-(2,4-dichlorobenzyl)pyrrolidin-3-yl)methyl)-2-oxo-2,3-dihydro-1H-benzo[d]imidazole-5-carboxylate

A mixture of methyl 2-oxo-3-(pyrrolidin-3-ylmethyl)-2,3-dihydro-1H-benzo[d]imidazole-5-carboxylate hydrochloride (50.0 mg, 0.160 mmol), 2,4-dichlorobenzaldehyde (35.1 mg, 0.201 mmol) and sodium triacetoxyborohydride (102 mg, 0.481 mmol) in chloroform (5 mL) was stirred at room temperature for 18 h. The contents were treated with 5% Na₂CO₃ and extracted with CHCl₃ (3×). The organic layers were filtered through a cotton plug, reduced in volume with a stream of N₂ and then purified by automated normal-phase chromatography (0-20% MeOH/DCM, 4 g silica gel cartridge) to give methyl 3-((1-(2,4-dichlorobenzyl)pyrrolidin-3-yl)methyl)-2-oxo-2,3-dihydro-1H-benzo[d]imidazole-5-carboxylate (44.6 mg, 0.103 mmol, 64.0% yield) as an off-white solid.

¹H NMR (400 MHz, DMSO-d₆) δ ppm 11.33 (br. s., 1H) 7.67-7.71 (m, 2H) 7.57 (d, J=2.27 Hz, 1H) 7.54 (d, J=8.34 Hz, 1H) 7.39 (dd, J=8.21, 2.15 Hz, 1H) 7.08 (d, J=8.84 Hz, 1H) 3.78-3.90 (m, 4H) 3.58-3.70 (m, 2H) 2.61-2.71 (m, 2H) 2.40-2.48 (m, 2H) 1.81-1.93 (m, 1H) 1.55 (td, J=13.20, 6.44 Hz, 1H)

LCMS: [M+1]=434/436/438, rt=1.95 min.

### 3-((1-(2,4-dichlorobenzyl)pyrrolidin-3-yl)methyl)-2-oxo-2,3-dihydro-1H-benzo[d]imidazole-5-carboxylic acid

To a suspension of methyl 3-((1-(2,4-dichlorobenzyl)pyrrolidin-3-yl)methyl)-2-oxo-2,3-dihydro-1H-benzo[d]imidazole-5-carboxylate (44.6 mg, 0.103 mmol) in methanol (5 mL) was added 1 N NaOH (1.03 mL, 1.03 mmol). The resulting mixture was stirred at 60° C. for 46 h. The solvent was removed under a stream of N₂, and the residue taken up in water and filtered through a syringe filter. The solution was acidified by addition of 6 N HCl and stirred for 18 h. The solid was collected by filtration, washed with water and dried under vacuum to give 3-((1-(2,4-dichlorobenzyl)pyrrolidin-3-yl)methyl)-2-oxo-2,3-dihydro-1H-benzo[d]imidazole-5-carboxylic acid hydrochloride (33.7 mg, 0.0738 mmol, 71.9% yield) as a white solid.

¹H NMR (400 MHz, DMSO-d₆) δ ppm 12.76 (br. s., 1H) 11.35 (s, 1H) 10.37 (br. s., 1H) 7.75-7.83 (m, 3H) 7.70 (d, J=8.08 Hz, 1H) 7.55-7.63 (m, 1H) 7.08 (d, J=8.08 Hz, 1H) 4.48-4.56 (m, 2H) 3.93-4.07 (m, 2H) 3.44-3.61 (m, 2H) 2.97-3.14 (m, 1H) 2.14 (br. s., 1H) 2.01 (d, J=8.84 Hz, 1H) 1.91 (br. s., 1H) 1.68-1.81 (m, 1H)

LCMS: [M+1]=420/422/424, rt=1.73 min.

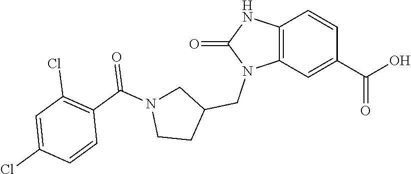 [SMILES: O=C(O)c1ccc2[nH]c(=O)n(CC3CCN(C(=O)c4ccc(Cl)cc4Cl)C3)c2c1]

### Example 19: 3-((1-(2,4-dichlorobenzoyl)pyrrolidin-3-yl)methyl)-2-oxo-2,3-dihydro-1H-benzo[d]imidazole-5-carboxylic acid

### Methyl 3-((1-(2,4-dichlorobenzoyl)pyrrolidin-3-yl)methyl)-2-oxo-2,3-dihydro-1H-benzo[d]imidazole-5-carboxylate

A mixture of methyl 2-oxo-3-(pyrrolidin-3-ylmethyl)-2,3-dihydro-1H-benzo[d]imidazole-5-carboxylate hydrochloride (50.0 mg, 0.160 mmol), 2,4-dichlorobenzoic acid (36.8 mg, 0.193 mmol) HBTU (73.0 mg, 0.193 mmol) and DIPEA (112 μL, 0.642 mmol) in chloroform (5 mL) was stirred at room temperature for 16 h. The contents were treated with 5% Na₂CO₃ and extracted with CHCl₃ (3×). The organic layers were filtered through a cotton plug, reduced in volume with a stream of N₂ and then purified by automated normal-phase chromatography (0-20% MeOH/DCM, 4 g silica gel cartridge) to give methyl 3-((1-(2,4-dichlorobenzoyl)pyrrolidin-3-yl)methyl)-2-oxo-2,3-dihydro-1H-benzo[d]imidazole-5-carboxylate (55.6 mg, 0.124 mmol, 77.3% yield) as a colorless gum. Two amide conformers by ¹H NMR—one peak by LCMS

¹H NMR (400 MHz, DMSO-d₆) δ ppm 11.36 (br. s., 1H) 7.67-7.80 (m, 3H) 7.42-7.55 (m, 2H) 7.09 (dd, J=14.91, 8.08 Hz, 1H) 3.94-3.99 (m, 1H) 3.87 (d, J=7.07 Hz, 1H) 3.84 (d, J=2.02 Hz, 3H) 3.56-3.69 (m, 2H) 3.44 (d, J=12.38 Hz, 1H) 2.96-3.29 (m, 2H) 1.86-2.01 (m, 1H) 1.64-1.79 (m, 1H)

LCMS: [M+1]=448/450/452, rt=2.27 min.

### 3-((1-(2,4-dichlorobenzoyl)pyrrolidin-3-yl)methyl)-2-oxo-2,3-dihydro-1H-benzo[d]imidazole-5-carboxylic acid

To a suspension of methyl 3-((1-(2,4-dichlorobenzoyl)pyrrolidin-3-yl)methyl)-2-oxo-2,3-dihydro-1H-benzo[d]imidazole-5-carboxylate (55.6 mg, 0.124 mmol) in methanol (5 mL) was added 1 N NaOH (1.24 mL, 1.24 mmol). The resulting mixture was stirred at 60° C. for 46 h. The solvent was removed under a stream of N₂, and the residue taken up in water and filtered through a syringe filter. The solution was acidified by addition of 6 N HCl and stirred for 18 h. The solid was collected by filtration, washed with water and dried under vacuum to give 3-((1-(2,4-dichlorobenzoyl)pyrrolidin-3-yl)methyl)-2-oxo-2,3-dihydro-1H-benzo[d]imidazole-5-carboxylic acid (31.4 mg, 0.0723 mmol, 58.3% yield) as a white solid. ¹H NMR shows two amide conformers—one peak by LCMS.

¹H NMR (400 MHz, DMSO-d₆) δ ppm 12.74 (br. s., 1H) 11.27-11.34 (m, 1H) 7.64-7.79 (m, 3H) 7.43-7.54 (m, 2H) 7.06 (dd, J=14.65, 8.08 Hz, 1H) 3.82-4.00 (m, 2H) 3.41-3.69 (m, 2H) 2.96-3.31 (m, 2H) 2.65-2.80 (m, 1H) 1.86-2.04 (m, 1H) 1.63-1.80 (m, 1H)

LCMS: [M+1]=420/422/424, rt=1.96 min.

### IP6K1 IC₅₀ Values of Representative Compounds of Formula (IA) and Formula (IB)

Representative IP6K1 IC₅₀ values of compounds of formula (IA) and formula (IB) are provided in Table 1 and Table 2.

TABLE 1
Representative IP6K1 IC50 of Compounds of Formula (IA)

| | | IP6K1 IC₅₀ |
|---|---|---|
| | | (<300 nM, +++) |
| Cmpd | | (300-3000 nM, ++) |
| No. | IUPAC name | (3000-30000 nM, +) |
| 1a | 1′-((4-chlorophenyl)sulfonyl)-2-oxospiro[indoline-3,4′- | + + |
| | piperidine]-5-carboxylic acid | |
| 2a | 1′-(4-chlorobenzoyl)-2-oxospiro[indoline-3,4′-piperidine]- | + + + |
| | 5-carboxylic acid | |
| 3a | methyl 1′-(4-chlorobenzoyl)-2-oxospiro[indoline-3,4′- | + |
| | piperidine]-5-carboxylate | |
| 4a | 1′-(2-(4-chlorophenyl)acetyl)-2-oxospiro[indoline-3,4′- | + + |
| | piperidine]-5-carboxylic acid | |
| 5a | 1′-(4-chlorobenzyl)-2-oxospiro[indoline-3,4′-piperidine]- | + + |
| | 5-carboxylic acid | |
| 6a | 1′-(3,5-dichloropicolinoyl)-2-oxospiro[indoline-3,4′- | + + + |
| | piperidine]-5-carboxylic acid | |
| 7a | 1′-((4-chlorophenyl)carbamoyl)-2-oxospiro[indoline-3,4′- | + + + |
| | piperidine]-5-carboxylic acid | |
| 8a | 2-oxo-1′-(4-(trifluoromethyl)benzoyl)spiro[indoline-3,4′- | + + + |
| | piperidine]-5-carboxylic acid | |
| 9a | 1′-(2-(4-chlorophenoxy)acetyl)-2-oxospiro[indoline-3,4′- | + + |
| | piperidine]-5-carboxylic acid | |
| 10a | 1′-(cyclopropanecarbonyl)-2-oxospiro[indoline-3,4′- | + |
| | piperidine]-5-carboxylic acid | |
| 11a | 1′-(4-chlorobenzoyl)-1-methyl-2-oxospiro[indoline-3,4′- | + |
| | piperidine]-5-carboxylic acid | |
| 12a | 1′-acetyl-2-oxospiro[indoline-3,4′-piperidine]-5- | + |
| | carboxylic acid | |
| 13a | 1′-isobutyryl-2-oxospiro[indoline-3,4′-piperidine]-5- | + |
| | carboxylic acid | |
| 14a | 1′-(2-chlorobenzoyl)-2-oxospiro[indoline-3,4′-piperidine]- | + + |
| | 5-carboxylic acid | |
| 15a | 1′-(2-chlorobenzyl)-2-oxospiro[indoline-3,4′-piperidine]- | + + |
| | 5-carboxylic acid | |
| 16a | 1′-(2,4-dichlorobenzyl)-2-oxospiro[indoline-3,4′- | + + + |
| | piperidine]-5-carboxylic acid | |
| 17a | 1′-(2,4-dichlorobenzoyl)-2-oxospiro[indoline-3,4′- | + + + |
| | piperidine]-5-carboxylic acid | |
| 18a | 1′-((3,5-dichloropyridin-2-yl)methyl)-2-oxospiro[indoline- | + + + |
| | 3,4′-piperidine]-5-carboxylic acid | |
| 1b | 3-(1-(2-chlorobenzoyl)piperidin-4-yl)-2-oxo-2,3- | + + |
| | dihydro-1H-benzo[d]imidazole-5-carboxylic acid | |
| 2b | 3-(1-(2-chlorobenzyl)piperidin-4-yl)-2-oxo-2,3-dihydro- | + + |
| | 1H-benzo[d]imidazole-5-carboxylic acid | |
| 3b | 3-(1-(2,4-dichlorobenzoyl)piperidin-4-yl)-2-oxo-2,3- | + + + |
| | dihydro-1H-benzo[d]imidazole-5-carboxylic acid | |
| 5b | 3-((1r,4r)-4-((3,5-dichloropyridin-2-yl)oxy)cyclohexyl)- | + + + |
| | 2-oxo-2,3-dihydro-1H-benzo[d]imidazole-5-carboxylic | |
| | acid | |
| 4b | 3-(1-(2,4-dichlorobenzyl)piperidin-4-yl)-2-oxo-2,3- | + + |
| | dihydro-1H-benzo[d]imidazole-5-carboxylic acid | |
| 6b | 3-((1s,4s)-4-((3,5-dichloropyridin-2-yl)oxy)cyclohexyl)- | + + + |
| | 2-oxo-2,3-dihydro-1H-benzo[d]imidazole-5-carboxylic | |
| | acid | |
| 8b | 3-(2-(4-chlorophenoxy)ethyl)-2-oxo-2,3-dihydro-1H- | + + |
| | benzo[d]imidazole-5-carboxylic acid | |
| 10b | 3-(1-(2,4-dichlorobenzoyl)azetidin-3-yl)-2-oxo-2,3- | + + |
| | dihydro-1H-benzo[d]imidazole-5-carboxylic acid | |
| 11b | 3-(1-(2,4-dichlorobenzyl)azetidin-3-yl)-2-oxo-2,3- | + + |
| | dihydro-1H-benzo[d]imidazole-5-carboxylic acid | |
| 12b | (S)-3-(1-(2,4-dichlorobenzoyl)pyrrolidin-3-yl)-2-oxo-2,3- | + + |
| | dihydro-1H-benzo[d]imidazole-5-carboxylic acid | |
| 14b | (R)-3-(1-(2,4-dichlorobenzoyl)pyrrolidin-3-yl)-2-oxo- | + + |
| | 2,3-dihydro-1H-benzo[d]imidazole-5-carboxylic acid | |
| 16b | 1-(1-(2,4-dichlorobenzoyl)piperidin-4-yl)-2-oxo-2,3- | + + |
| | dihydro-1H-benzo[d]imidazole-5-carboxylic acid | |
| 13b | (S)-3-(1-(2,4-dichlorobenzyl)pyrrolidin-3-yl)-2-oxo-2,3- | + + |
| | dihydro-1H-benzo[d]imidazole-5-carboxylic acid | |
| 15b | (R)-3-(1-(2,4-dichlorobenzyl)pyrrolidin-3-yl)-2-oxo-2,3- | + + |
| | dihydro-1H-benzo[d]imidazole-5-carboxylic acid | |
| 17b | 1-(1-(2,4-dichlorobenzyl)piperidin-4-yl)-2-oxo-2,3- | + |
| | dihydro-1H-benzo[d]imidazole-5-carboxylic acid | |
| 9b | 3-(3-((3,5-dichloropyridin-2-yl)oxy)propyl)-2-oxo-2,3- | + + + |
| | dihydro-1H-benzo[d]imidazole-5-carboxylic acid | |
| 18b | 3-((1-(2,4-dichlorobenzyl)pyrrolidin-3-yl)methyl)-2-oxo- | + |
| | 2,3-dihydro-1H-benzo[d]imidazole-5-carboxylic acid | |
| 19b | 3-((1-(2,4-dichlorobenzoyl)pyrrolidin-3-yl)methyl)-2- | + + |
| | oxo-2,3-dihydro-1H-benzo[d]imidazole-5-carboxylic | |
| | acid | |
| 7b | 3-((1r,4r)-4-((3,5-dichloropyridin-2-yl)oxy)cyclohexyl)- | + + + |
| | 6-fluoro-2-oxo-2,3-dihydro-1H-benzo[d]imidazole-5- | |
| | carboxylic acid | |

### REFERENCES

Chakraborty A, Koldobskiy M A, Bello N T, et al. Inositol Pyrophosphates Inhibit Akt Signaling, Thereby Regulating Insulin Sensitivity and Weight Gain. Cell. 2010; 143(6):897-910.
Chakraborty A, Latapy C, Xu J, Snyder S H, Beaulieu J-M. Inositol hexakisphosphate kinase-1 regulates behavioral responses via GSK3 signaling pathways. Mol Psychiatry. 2014; 19:284-293.
Beurel E, Grieco S F, Jope R S. Glycogen synthase kinase-3 (GSK3): regulation, actions, and diseases. Pharmacol Ther. 2015; 148:114-131.
Beaulieu J M. A role for Akt and glycogen synthase kinase-3 as integrators of dopamine and serotonin neurotransmission in mental health. J Psychiatry Neurosci. 2012; 37(1):7-16.
Tan H Y, Chen A G, Kolachana B, et al. Effective connectivity of AKT1-mediated dopaminergic working memory networks and pharmacogenetics of anti-dopaminergic treatment. Brain. 2012; 135(5):1436-1445.
Emamian E S, Hall D, Birnbaum M J, Karayiorgou M, Gogos J A. Convergent evidence for impaired AKT1-GSK30 signaling in schizophrenia. Nat Genet. 2004; 36(2):131-137.
Bhat R V., Andersson U, Andersson S, Knerr L, Bauer U, Sundgren-Andersson A K. The Conundrum of GSK3 Inhibitors: Is it the Dawn of a New Beginning? Perry G, Avila J, Moreira P I, Sorensen A A, Tabaton M, eds. J Alzheimer's Dis. 2018; 64(s1):5547-5554.
Jope R S. Lithium and GSK-3: one inhibitor, two inhibitory actions, multiple outcomes. Trends Pharmacol Sci. 2003; 24(9):441-443.
Beaulieu J-M, Marion S, Rodriguiz R M, et al. A β-arrestin 2 Signaling Complex Mediates Lithium Action on Behavior. Cell. 2008; 132(1):125-136.
Bowden C L. Efficacy of lithium in mania and maintenance therapy of bipolar disorder. J Clin Psychiatry. 2000; 61 Suppl 9:35-40.
Le Roy V, Delmas Y, Verdoux H. Chronic renal complications induced by lithium. Encephale. 2009; 35(6):605-610.
Pan J Q, Lewis M C, Ketterman J K, et al. AKT Kinase Activity Is Required for Lithium to Modulate Mood-Related Behaviors in Mice. Neuropsychopharmacology. 2011; 36(7):1397-1411.
Gould T D, O'Donnell K C, Picchini A M, Manji H K. Strain Differences in Lithium Attenuation of d-Amphetamine-Induced Hyperlocomotion: A Mouse Model for the Genetics of Clinical Response to Lithium. Neuropsychopharmacology. 2007; 32(6):1321-1333.
Miller, E., et al., Type 2 diabetes: Evolving concepts and treatment. Cleve Clin J Med, 2019. 86(7): p. 494-504.
Petersen, M. C. and G. I. Shulman, Mechanisms of Insulin Action and Insulin Resistance. Physiol Rev, 2018. 98(4): p. 2133-2223.
Hickson, R. P., A. L. Cole, and S. B. Dusetzina, Implications of Removing Rosiglitazone's Black Box Warning and Restricted Access Program on the Uptake of Thiazolidinediones and Dipeptidyl Peptidase-4 Inhibitors Among Patients with Type 2 Diabetes. J Manag Care Spec Pharm, 2019. 25(1): p. 72-79.
Marks, D. H., Drug utilization, safety and clinical use of Actos and Avandia. Int J Risk Saf Med, 2013. 25(1): p. 39-51.
Fayard, E., et al., Protein Kinase B (PKB/Akt), a Key Mediator of the PI3K Signaling Pathway. Phosphoinositide 3-Kinase in Health and Disease, Vol 1, 2010. 346: p. 31-56.
Ghoshal, S., et al., TNP [N₂-(m-Trifluorobenzyl), N₆-(p-nitrobenzyl)purine]ameliorates diet induced obesity and insulin resistance via inhibition of the IP6K1 pathway. Mol Metab, 2016. 5(10): p. 903-17.
Barclay, R. D., et al., Ingestion of lean meat elevates muscle inositol hexakisphosphate kinase 1 protein content independent of a distinct post-prandial circulating proteome in young adults with obesity. Metabolism, 2019: p. 153996.
Zhu, Q., et al., Adipocyte-specific deletion of Ip6k1 reduces diet-induced obesity by enhancing AMPK-mediated thermogenesis. J Clin Invest, 2016.
Cokorinos, E. C., et al., Activation of Skeletal Muscle AMPK Promotes Glucose Disposal and Glucose Lowering in Non-human Primates and Mice. Cell Metab, 2017. 25(5): p. 1147-1159 e10.
Myers, R. W., et al., Systemic pan-AMPK activator MK-8722 improves glucose homeostasis but induces cardiac hypertrophy. Science, 2017. 357(6350): p. 507-511.
Steneberg, P., et al., PAN-AMPK activator 0304 improves glucose homeostasis and microvascular perfusion in mice and type 2 diabetes patients. JCI Insight, 2018. 3(12).
Gu, C., et al., K O of 5-InsP7 kinase activity transforms the HCT116 colon cancer cell line into a hypermetabolic, growth-inhibited phenotype. Proc Natl Acad Sci USA, 2017. 114(45): p. 11968-11973.
Szijgyarto, Z., et al., Influence of inositol pyrophosphates on cellular energy dynamics. Science, 2011. 334(6057): p. 802-5.
Sun, D., et al., Oncostatin M (OSM) protects against cardiac ischaemia/reperfusion injury in diabetic mice by regulating apoptosis, mitochondrial biogenesis and insulin sensitivity. J Cell Mol Med, 2015. 19(6): p. 1296-307.
Szendroedi, J., et al., Muscle mitochondrial ATP synthesis and glucose transport/phosphorylation in type 2 diabetes. PLoS Med, 2007. 4(5): p. e154.
Koliaki, C. and M. Roden, Hepatic energy metabolism in human diabetes mellitus, obesity and non-alcoholic fatty liver disease. Mol Cell Endocrinol, 2013. 379(1-2): p. 35-42.
Muller, F., et al., Platelet polyphosphates are proinflammatory and procoagulant mediators in vivo. Cell, 2009. 139(6): p. 1143-56.
Labberton, L., et al., Neutralizing blood-borne polyphosphate in vivo provides safe thromboprotection. Nat Commun, 2016. 7: p. 12616.
Ghosh, S., et al., Inositol hexakisphosphate kinase 1 maintains hemostasis in mice by regulating platelet polyphosphate levels. Blood, 2013. 122(8): p. 1478-1486.
Booth, G. L., et al., Relation between age and cardiovascular disease in men and women with diabetes compared with non-diabetic people: a population-based retrospective cohort study. Lancet, 2006. 368(9529): p. 29-36.
Authors/Task Force, M., et al., ESC Guidelines on diabetes, pre-diabetes, and cardiovascular diseases developed in collaboration with the EASD: the Task Force on diabetes, pre-diabetes, and cardiovascular diseases of the European Society of Cardiology (ESC) and developed in collaboration with the European Association for the Study of Diabetes (EASD). Eur Heart J, 2013. 34(39): p. 3035-87.
International PCT Patent Application Publication No. WO2018182051 for IP6K INHIBITORS to Terao et al., published Oct. 4, 2018.

Although the foregoing subject matter has been described in some detail by way of illustration and example for purposes of clarity of understanding, it will be understood by those skilled in the art that certain changes and modifications can be practiced within the scope of the appended claims.

1. A compound of formula (IA): 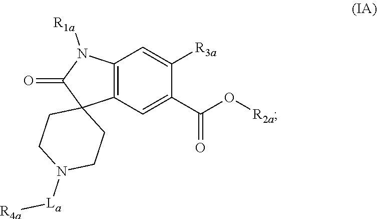
wherein:
R₁ₐ and R₂ₐ are each independently selected from the group consisting of H and substituted or unsubstituted branched or straightchain C₁-C₄ alkyl, provided that at least one of R₁ₐ, and R₂ₐ, is H;
R₃ₐ is H or halogen;
R₄ₐ is selected from the group consisting of substituted or unsubstituted branched or straightchain C₁-C₄ alkyl, substituted or unsubstituted cycloalkyl, substituted or unsubstituted cycloheteroalkyl, substituted or unsubstituted aryl, and substituted or unsubstituted heteroaryl;
Lₐ is selected from the group consisting of —(CH₂)ₙ—, —S(═O)₂—, —C(═O)—(CH₂)ₘ—, —C(═O)—NH—, and —C(═O)—(CH₂)ₚ—O—;
wherein n and p are each integers selected from 1, 2, 3, and 4; m is 0 or 1; and
stereoisomers and pharmaceutically acceptable salts thereof.

2. The compound of claim 1, wherein R₁ₐ and R₂ₐ are each independently H or methyl, provided that at least one of R₁ₐ and R₂ₐ, is H.

3. The compound of claim 1, wherein R₃ₐ is H or F.

4. The compound of claim 1, wherein R₄ₐ is selected from the group consisting of methyl, isopropyl, cyclopropyl, substituted or unsubstituted phenyl, substituted or unsubstituted pyridyl, and substituted or unsubstituted pyrimidinyl.

5. The compound of claim 4, wherein the phenyl, pyridinyl, or pyrimidinyl is substituted with one or more substituent groups selected from the group consisting of substituted or unsubstituted branched or straightchain C₁-C₄ alkyl, C₁-C₄ alkoxyl, cyclopropyl, trifluoromethyl, hydroxyl, halogen, cyano, carbamoyl, and benzyloxy.

6. The compound of claim 4, wherein R₄ₐ is selected from the group consisting of 2-chlorophenyl, 4-chlorophenyl, 2,4-dichlorophenyl, 4-trifluoromethylphenyl, and 3,5-dichloropyridin-2-yl.

7. The compound of claim 1, wherein the compound of formula (IA) is selected from the group consisting of:
1′-((4-chlorophenyl)sulfonyl)-2-oxospiro[indoline-3,4′-piperidine]-5-carboxylic acid;
1′-(4-chlorobenzoyl)-2-oxospiro[indoline-3,4′-piperidine]-5-carboxylic acid;
methyl 1′-(4-chlorobenzoyl)-2-oxospiro[indoline-3,4′-piperidine]-5-carboxylate;
1′-(2-(4-chlorophenyl)acetyl)-2-oxospiro[indoline-3,4′-piperidine]-5-carboxylic acid;
1′-(4-chlorobenzyl)-2-oxospiro[indoline-3,4′-piperidine]-5-carboxylic acid;
1′-(3,5-dichloropicolinoyl)-2-oxospiro[indoline-3,4′-piperidine]-5-carboxylic acid;
1′-((4-chlorophenyl)carbamoyl)-2-oxospiro[indoline-3,4′-piperidine]-5-carboxylic acid;
2-oxo-1′-(4-(trifluoromethyl)benzoyl)spiro[indoline-3,4′-piperidine]-5-carboxylic acid;
1′-(2-(4-chlorophenoxy)acetyl)-2-oxospiro[indoline-3,4′-piperidine]-5-carboxylic acid;
1′-(cyclopropanecarbonyl)-2-oxospiro[indoline-3,4′-piperidine]-5-carboxylic acid;
1′-(4-chlorobenzoyl)-1-methyl-2-oxospiro[indoline-3,4′-piperidine]-5-carboxylic acid;
1′-acetyl-2-oxospiro[indoline-3,4′-piperidine]-5-carboxylic acid;
1′-isobutyryl-2-oxospiro[indoline-3,4′-piperidine]-5-carboxylic acid;
1′-(2-chlorobenzoyl)-2-oxospiro[indolineho-3,4′-piperidine]-5-carboxylic acid;
1′-(2-chlorobenzyl)-2-oxospiro[indoline-3,4′-piperidine]-5-carboxylic acid;
1′-(2,4-dichlorobenzyl)-2-oxospiro[indoline-3,4′-piperidine]-5-carboxylic acid;
1′-(2,4-dichlorobenzoyl)-2-oxospiro[indoline-3,4′-piperidine]-5-carboxylic acid;
1′-((3,5-dichloropyridin-2-yl)methyl)-2-oxospiro[indoline-3,4′-piperidine]-5-carboxylic acid;
and stereoisomers and pharmaceutically acceptable salts thereof.

8. A compound of formula (IB): 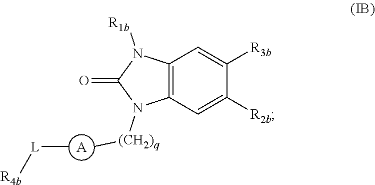
wherein:
q is 0 or 1;
A can be present or absent and when present is a 4-, 5-, or 6-membered cycloalkyl or cycloheteroalkyl ring;
R_{1b} is selected from the group consisting of H and substituted or unsubstituted branched or straightchain C₁-C₄ alkyl;
R_{2b} is H or —C(═O)—OR_{5b};
R_{3b} is selected from the group consisting of H, halogen, and —C(═O)—OR_{5b}, wherein R_{5b} is selected from the group consisting of H and substituted or unsubstituted branched or straightchain C₁-C₄ alkyl, provided that at least one of R_{1b} and R_{5b} is H;
R_{4b} is selected from the group consisting of substituted or unsubstituted branched or straightchain C₁-C₄ alkyl, substituted or unsubstituted cycloalkyl, substituted or unsubstituted cycloheteroalkyl, substituted or unsubstituted aryl, and substituted or unsubstituted heteroaryl;
L_{b} is selected from the group consisting of —C(═O)—, —(CH₂)ᵣ—, and —(CH₂)ₛ—O—; wherein r is selected from the group consisting of 1, 2, 3, or 4 and s is an integer selected from the group consisting of 0, 1, 2, or 3; and
stereoisomers and pharmaceutically acceptable salts thereof.

9. The compound of claim 8, wherein the compound of formula (IB) is:

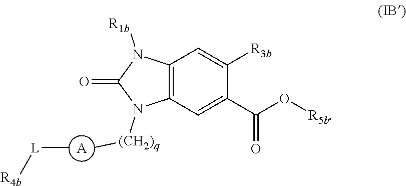

10. The compound of claim 9, wherein R_{1b} and R_{5b} are each H and/or R_{3b} is H or F.

11. (canceled)

12. The compound of claim 8, wherein A is present and is selected from the group consisting of azetidinyl, pyrrolidinyl, piperidinyl, and cyclohexyl: 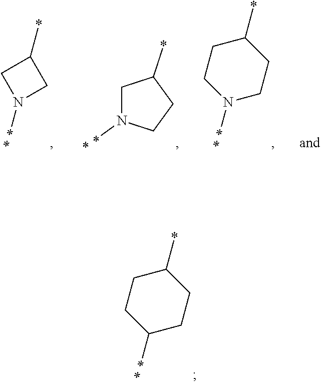
wherein * indicates a point of attachment of ring A to a nitrogen atom in the 3-position of the 2,3-dihydro-1H-benzo[d]imidazole ring and ** indicates a point of attachment of ring A to linker L_{b}.

13. The compound of claim 8, wherein R_{4b} is selected from the group consisting of methyl, isopropyl, cyclopropyl, substituted or unsubstituted phenyl, substituted or unsubstituted pyridyl, and substituted or unsubstituted pyrimidinyl, wherein the phenyl, pyridinyl, or pyrimidinyl optionally is substituted with one or more substituent groups selected from the group consisting of substituted or unsubstituted branched or straightchain C₁-C₄ alkyl, C₁-C₄ alkoxyl, cyclopropyl, trifluoromethyl, hydroxyl, halogen, cyano, carbamoyl, and benzyloxy.

14.-15. (canceled)

16. The compound of claim 13, wherein R_{4b} is selected from the group consisting of 2-chlorophenyl, 4-chlorophenyl, 2,4-dichlorophenyl, and 3,5-dichloropyridin-2-yl.

17. The compound of claim 8, wherein the compound of formula (IB) is selected from the group consisting of:
3-(1-(2-chlorobenzoyl)piperidin-4-yl)-2-oxo-2,3-dihydro-1H-benzo[d]imidazole-5-carboxylic acid;
3-(1-(2-chlorobenzyl)piperidin-4-yl)-2-oxo-2,3-dihydro-1H-benzo[d]imidazole-5-carboxylic acid;
3-(1-(2,4-dichlorobenzoyl)piperidin-4-yl)-2-oxo-2,3-dihydro-1H-benzo[d]imidazole-5-carboxylic acid;
3-((1r,4r)-4-((3,5-dichloropyridin-2-yl)oxy)cyclohexyl)-2-oxo-2,3-dihydro-1H-benzo[d]imidazole-5-carboxylic acid;
3-(1-(2,4-dichlorobenzyl)piperidin-4-yl)-2-oxo-2,3-dihydro-1H-benzo[d]imidazole-5-carboxylic acid;
3-((1s,4s)-4-((3,5-dichloropyridin-2-yl)oxy)cyclohexyl)-2-oxo-2,3-dihydro-1H-benzo[d]imidazole-5-carboxylic acid;
3-(2-(4-chlorophenoxy)ethyl)-2-oxo-2,3-dihydro-1H-benzo[d]imidazole-5-carboxylic acid;
3-(1-(2,4-dichlorobenzoyl)azetidin-3-yl)-2-oxo-2,3-dihydro-1H-benzo[d]imidazole-5-carboxylic acid;
3-(1-(2,4-dichlorobenzyl)azetidin-3-yl)-2-oxo-2,3-dihydro-1H-benzo[d]imidazole-5-carboxylic acid;
(S)-3-(1-(2,4-dichlorobenzoyl)pyrrolidin-3-yl)-2-oxo-2,3-dihydro-1H-benzo[d]imidazole-5-carboxylic acid;
(R)-3-(1-(2,4-dichlorobenzoyl)pyrrolidin-3-yl)-2-oxo-2,3-dihydro-1H-benzo[d]imidazole-5-carboxylic acid;
1-(1-(2,4-dichlorobenzoyl)piperidin-4-yl)-2-oxo-2,3-dihydro-1H-benzo[d]imidazole-5-carboxylic acid;
(S)-3-(1-(2,4-dichlorobenzyl)pyrrolidin-3-yl)-2-oxo-2,3-dihydro-1H-benzo[d]imidazole-5-carboxylic acid;
(R)-3-(1-(2,4-dichlorobenzyl)pyrrolidin-3-yl)-2-oxo-2,3-dihydro-1H-benzo[d]imidazole-5-carboxylic acid;
1-(1-(2,4-dichlorobenzyl)piperidin-4-yl)-2-oxo-2,3-dihydro-1H-benzo[d]imidazole-5-carboxylic acid;
3-(3-((3,5-dichloropyridin-2-yl)oxy)propyl)-2-oxo-2,3-dihydro-1H-benzo[d]imidazole-5-carboxylic acid;
3-((1-(2,4-dichlorobenzyl)pyrrolidin-3-yl)methyl)-2-oxo-2,3-dihydro-1H-benzo[d]imidazole-5-carboxylic acid;
3-((1-(2,4-dichlorobenzoyl)pyrrolidin-3-yl)methyl)-2-oxo-2,3-dihydro-1H-benzo[d]imidazole-5-carboxylic acid;
3-((1r,4r)-4-((3,5-dichloropyridin-2-yl)oxy)cyclohexyl)-6-fluoro-2-oxo-2,3-dihydro-1H-benzo[d]imidazole-5-carboxylic acid; and
stereoisomers and pharmaceutically acceptable salts thereof.

18. A method for treating a disease, condition, or disorder associated with IP6K, the method comprising administering a compound of claim 1, or a pharmaceutically acceptable salt thereof, to a subject in need of such treatment.

19. The method of claim 18, wherein the disease, condition, or disorder is selected from the group consisting of a psychiatric disease, Alzheimer's disease, and diabetes.

20. The method of claim 19, wherein the psychiatric disease is bipolar disorder.

21. The method of claim 18, further comprising one or more of inhibiting IP6K, increasing AKT activity, and inhibiting GSK3 activity.

22. A method for treating a disease, condition, or disorder associated with IP6K, the method comprising administering a compound of claim 8, or a pharmaceutically acceptable salt thereof, to a subject in need of such treatment, wherein the method optionally comprises one or more of inhibiting IP6K, increasing AKT activity, and inhibiting GSK3 activity.

23. The method of claim 22, wherein the disease, condition, or disorder is selected from the group consisting of a psychiatric disease, Alzheimer's disease, and diabetes, wherein the psychiatric disease optionally is bipolar disorder.
